SEQUENCE LISTING (71 sequences)

SEQ ID NO: 1 - DNA, 5034 bp, Artificial Sequence
  Synthetic construct
atgcccatgggcagcctgcagcccctggccaccctgtacctgctgggcatgctggtggctagcgtgctggccgccgaaaacctgtgggtcaccgtgtattatggagtgcccgtctggaaagatgctgaaactaccctgttctgtgcctctgatgctaaggcctacgagaccgaaaagcacaatgtctgggctactcatgcatgcgtgcccaccgacccaaacccccaggagatccacctggaaaatgtgaccgaggaattcaacatgtggaaaaacaatatggtggagcagatgcatacagacatcattagcctgtgggatcagtccctgaagccctgcgtcaaactgactcctctgtgcgtgaccctgcagtgtaccaatgtcacaaacaatatcaccgacgatatgaggggcgagctgaagaattgtagcttcaacatgaccacagaactgagagacaagaaacagaaagtgtactccctgttttataggctggatgtggtccagatcaatgagaaccaggggaatcggagcaacaattccaacaaggaatacagactgatcaattgcaacacttccgccattacccaggcttgtcctaaagtgtcttttgagcctatcccaattcattattgcgccccagctggcttcgccatcctgaagtgtaaagataagaagttcaacggaactggcccctgcccttccgtgtctacagtccagtgtactcacgggattaagcctgtggtctctacacagctgctgctgaatggaagtctggctgaggaagaagtgatgatccggagcgagaacattaccaacaatgccaagaatatcctggtccagttcaacacaccagtgcagattaattgcacaagacccaacaataacactcgaaaatctatccggattgggccaggacaggccttttacgctacaggggacatcattggagatatcagacaggctcactgtaccgtgagtaaggcaacctggaacgagacactgggcaaggtggtcaaacagctgaggaaacatttcgggaataacaccatcattcgctttgccaatagctccggaggggacctggaggtcactacccactccttcaactgcggaggcgaattcttttactgtaacacatctggcctgtttaatagtacatggatctctaacactagtgtgcagggcagtaattcaactgggtcaaacgatagcatcaccctgccatgccgaattaagcagatcattaatatgtggcagcggatcggccaggcaatgtatgccccccctatccagggggtcattcgctgcgtgagcaatatcaccggactgattctgacacgagacgggggcagcaccaactctacaactgaaacattccggcccggcgggggagacatgagagataactggaggtccgagctgtacaagtataaagtggtcaagatcgaacctctgggagtggcaccaaccagatgcaagcgaagagtggtcggaggcggcagcggcggcggcggctccggcggcggcggctctggcggcgcagtcggaattggggccgtgttcctgggatttctgggcgccgctgggagtacaatgggagcagcctcaatgactctgaccgtgcaggccaggaatctgctgagcggcatcgtccagcagcagtccaacctgctgcgcgctcctgaagcacagcagcacctgctgaagctgaccgtgtggggcatcaaacagctgcaggctagggtgctggcagtcgagcggtacctgagagaccagcagctgctgggaatctggggctgctctgggaagctgatttgttgcacaaatgtgccttggaactctagttggtcaaatcgcaacctgagcgagatctgggacaatatgacttggctgcagtgggataaagaaattagtaactacacccagatcatctacggcctgctggaagagtcacagaatcagcaggagaagaacgaacaggacctgctggcactggatggcagcggcgatatcatcaagctgctgaacgagcaagtgaataaggagatgcagagctccaacctgtacatgagcatgtctagctggtgctatacccactccctggacggagcaggactgttcctgtttgatcacgccgccgaggagtatgagcacgccaagaagctgatcatctttctgaatgagaacaatgtgcccgtgcagctgacctccatctctgcccctgagcacaagttcgagggcctgacacagatctttcagaaggcctacgagcacgagcagcacatcagcgagtccatcaacaatatcgtggaccacgccatcaagtccaaggatcacgccacattcaactttctgcagtggtacgtggccgagcagcacgaggaggaggtgctgttcaaggacatcctggataagatcgagctgatcggcaacgagaatcacggcctgtacctggccgaccagtatgtgaagggcatcgccaagtctcggaagagcggaagcggagctactaacttcagcctgctgaagcaggctggagacgtggaggagaaccctggacctggaagcggaatgcccatgggcagcctgcagcccctggccaccctgtacctgctgggcatgctggtggctagcgtgctggccgtgggcaacatgtgggtgacagtgtactatggcgtgcccgtgtggaccgatgccaagaccacactgttctgcgcctccgacacaaaggcctacgatcgggaggtgcacaacgtgtgggcaacacacgcatgcgtgccaaccgacccaaatccccaggagatcgtgctggagaacgtgaccgagaacttcaacatgtggaagaacgacatggtggatcagatgcacgaggacatcatcagcctgtgggatcagtccctgaagccatgcgtgaagctgacacccctgtgcgtgaccctgcactgtacaaacgccacctttaagaacaatgtgaccaatgatatgaacaaggagatcaggaattgttctttcaacaccacaaccgagatccgcgataagaagcagcagggctacgccctgttttataggcctgacatcgtgctgctgaaggagaatcgcaacaattctaacaatagcgagtatatcctgatcaattgcaacgccagcacaatcacccaggcctgtcccaaggtgaacttcgaccctatcccaatccactactgcgcccctgccggctatgccatcctgaagtgtaacaacaagaccttcagcggcaagggcccatgcaacaacgtgagcacagtgcagtgtacccacggcatcaagcccgtggtgtccacccagctgctgctgaatggctctctggccgagaaggagatcatcatcaggtccgagaatctgacagataacgtgaagaccatcatcgtgcacctgaacaagtccgtggagatcgtgtgcacacgccctaacaataacaccaggaagtctatgcgcatcggcccaggccagacattctacgccaccggcgacatcatcggcgatatccggcaggcctattgtaatatcagcggctccaagtggaacgagacactgaagagagtgaaggagaagctgcaggagaactacaataacaataagaccatcaagttcgcaccaagctccggaggcgatctggagatcacaacccacagctttaattgccggggcgagttcttttattgtaacacaaccagactgttcaacaataacgccaccgaggacgagacaatcaccctgccttgccggatcaagcagatcatcaatatgtggcagggagtgggaagagcaatgtacgcaccacctatcgccggcaatatcacctgtaagagcaacatcaccggactgctgctggtgagagacggaggagaggataacaagacagaggagatctttcggcccggcggcggcaatatgaaggacaactggagatccgagctgtacaagtataaagtgatcgagctgaagccactgggaatcgcacctaccggatgcaagaggagagtggtggagggaggctctggaggaggaggaagcggaggaggaggatccggcggcgccgtgggcatcggagccgtgttcctgggctttctgggagcagcaggatctaccatgggagcagcaagcctgacactgaccgtgcaggccaggcagctgctgtctagcatcgtgcagcagcagtccaatctgctgagggcaccagaggcacagcagcacatgctgcagctgacagtgtggggcatcaagcagctgcagacccgggtgctggccatcgagagatacctgaaggatcagcagctgctgggcatctggggctgctctggcaagctgatctgctgtaccaatgtgccctggaactcctcttggtccaacaagtctcagacagacatctggaataacatgacctggatggagtgggacagggagatctctaattacacagataccatctatcgcctgctggaggacagccagacccagcaggagaagaacgagaaggacctgctggccctggatggaagcggagatatcatcaagctgctgaacgagcaagtgaataaggagatgcagagctccaacctgtacatgagcatgtctagctggtgctatacccactccctggacggagcaggactgttcctgtttgatcacgccgccgaggagtatgagcacgccaagaagctgatcatctttctgaatgagaacaatgtgcccgtgcagctgacctccatctctgcccctgagcacaagttcgagggcctgacacagatctttcagaaggcctacgagcacgagcagcacatcagcgagtccatcaacaatatcgtggaccacgccatcaagtccaaggatcacgccacattcaactttctgcagtggtacgtggccgagcagcacgaggaggaggtgctgttcaaggacatcctggataagatcgagctgatcggcaacgagaatcacggcctgtacctggccgaccagtatgtgaagggcatcgccaagtctcggaagagc

SEQ ID NO: 2 - protein, 1678 aa, Artificial Sequence
  Synthetic construct
MPMGSLQPLATLYLLGMLVASVLAAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCTVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGGGSGGGGSGGGGSGGAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGSGATNFSLLKQAGDVEENPGPGSGMPMGSLQPLATLYLLGMLVASVLAVGNMWVTVYYGVPVWTDAKTTLFCASDTKAYDREVHNVWATHACVPTDPNPQEIVLENVTENFNMWKNDMVDQMHEDIISLWDQSLKPCVKLTPLCVTLHCTNATFKNNVTNDMNKEIRNCSFNTTTEIRDKKQQGYALFYRPDIVLLKENRNNSNNSEYILINCNASTITQACPKVNFDPIPIHYCAPAGYAILKCNNKTFSGKGPCNNVSTVQCTHGIKPVVSTQLLLNGSLAEKEIIIRSENLTDNVKTIIVHLNKSVEIVCTRPNNNTRKSMRIGPGQTFYATGDIIGDIRQAYCNISGSKWNETLKRVKEKLQENYNNNKTIKFAPSSGGDLEITTHSFNCRGEFFYCNTTRLFNNNATEDETITLPCRIKQIINMWQGVGRAMYAPPIAGNITCKSNITGLLLVRDGGEDNKTEEIFRPGGGNMKDNWRSELYKYKVIELKPLGIAPTGCKRRVVEGGSGGGGSGGGGSGGAVGIGAVFLGFLGAAGSTMGAASLTLTVQARQLLSSIVQQQSNLLRAPEAQQHMLQLTVWGIKQLQTRVLAIERYLKDQQLLGIWGCSGKLICCTNVPWNSSWSNKSQTDIWNNMTWMEWDREISNYTDTIYRLLEDSQTQQEKNEKDLLALDGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 3 - DNA, 4041 bp, Artificial Sequence
  Synthetic construct
atgcccatgggcagcctgcagcccctggccaccctgtacctgctgggcatgctggtggctagcgtgctggccgccgaaaacctgtgggtcaccgtgtattatggagtgcccgtctggaaagatgctgaaactaccctgttctgtgcctctgatgctaaggcctacgagaccgaaaagcacaatgtctgggctactcatgcatgcgtgcccaccgacccaaacccccaggagatccacctggaaaatgtgaccgaggaattcaacatgtggaaaaacaatatggtggagcagatgcatacagacatcattagcctgtgggatcagtccctgaagccctgcgtcaaactgactcctctgtgcgtgaccctgcagtgtaccaatgtcacaaacaatatcaccgacgatatgaggggcgagctgaagaattgtagcttcaacatgaccacagaactgagagacaagaaacagaaagtgtactccctgttttataggctggatgtggtccagatcaatgagaaccaggggaatcggagcaacaattccaacaaggaatacagactgatcaattgcaacacttccgccattacccaggcttgtcctaaagtgtcttttgagcctatcccaattcattattgcgccccagctggcttcgccatcctgaagtgtaaagataagaagttcaacggaactggcccctgcccttccgtgtctacagtccagtgtactcacgggattaagcctgtggtctctacacagctgctgctgaatggaagtctggctgaggaagaagtgatgatccggagcgagaacattaccaacaatgccaagaatatcctggtccagttcaacacaccagtgcagattaattgcacaagacccaacaataacactcgaaaatctatccggattgggccaggacaggccttttacgctacaggggacatcattggagatatcagacaggctcactgtaccgtgagtaaggcaacctggaacgagacactgggcaaggtggtcaaacagctgaggaaacatttcgggaataacaccatcattcgctttgccaatagctccggaggggacctggaggtcactacccactccttcaactgcggaggcgaattcttttactgtaacacatctggcctgtttaatagtacatggatctctaacactagtgtgcagggcagtaattcaactgggtcaaacgatagcatcaccctgccatgccgaattaagcagatcattaatatgtggcagcggatcggccaggcaatgtatgccccccctatccagggggtcattcgctgcgtgagcaatatcaccggactgattctgacacgagacgggggcagcaccaactctacaactgaaacattccggcccggcgggggagacatgagagataactggaggtccgagctgtacaagtataaagtggtcaagatcgaacctctgggagtggcaccaaccagatgcaagcgaagagtggtcggaggcggcagcggcggcggcggctccggcggcggcggctctggcggcgcagtcggaattggggccgtgttcctgggatttctgggcgccgctgggagtacaatgggagcagcctcaatgactctgaccgtgcaggccaggaatctgctgagcggcatcgtccagcagcagtccaacctgctgcgcgctcctgaagcacagcagcacctgctgaagctgaccgtgtggggcatcaaacagctgcaggctagggtgctggcagtcgagcggtacctgagagaccagcagctgctgggaatctggggctgctctgggaagctgatttgttgcacaaatgtgccttggaactctagttggtcaaatcgcaacctgagcgagatctgggacaatatgacttggctgcagtgggataaagaaattagtaactacacccagatcatctacggcctgctggaagagtcacagaatcagcaggagaagaacgaacaggacctgctggcactggatggcagcggcgctactaacttcagcctgctgaagcaggctggagacgtggaggagaaccctggacctggaagcggaatgcccatgggcagcctgcagcccctggccaccctgtacctgctgggcatgctggtggctagcgtgctggccgtgggcaacatgtgggtgacagtgtactatggcgtgcccgtgtggaccgatgccaagaccacactgttctgcgcctccgacacaaaggcctacgatcgggaggtgcacaacgtgtgggcaacacacgcatgcgtgccaaccgacccaaatccccaggagatcgtgctggagaacgtgaccgagaacttcaacatgtggaagaacgacatggtggatcagatgcacgaggacatcatcagcctgtgggatcagtccctgaagccatgcgtgaagctgacacccctgtgcgtgaccctgcactgtacaaacgccacctttaagaacaatgtgaccaatgatatgaacaaggagatcaggaattgttctttcaacaccacaaccgagatccgcgataagaagcagcagggctacgccctgttttataggcctgacatcgtgctgctgaaggagaatcgcaacaattctaacaatagcgagtatatcctgatcaattgcaacgccagcacaatcacccaggcctgtcccaaggtgaacttcgaccctatcccaatccactactgcgcccctgccggctatgccatcctgaagtgtaacaacaagaccttcagcggcaagggcccatgcaacaacgtgagcacagtgcagtgtacccacggcatcaagcccgtggtgtccacccagctgctgctgaatggctctctggccgagaaggagatcatcatcaggtccgagaatctgacagataacgtgaagaccatcatcgtgcacctgaacaagtccgtggagatcgtgtgcacacgccctaacaataacaccaggaagtctatgcgcatcggcccaggccagacattctacgccaccggcgacatcatcggcgatatccggcaggcctattgtaatatcagcggctccaagtggaacgagacactgaagagagtgaaggagaagctgcaggagaactacaataacaataagaccatcaagttcgcaccaagctccggaggcgatctggagatcacaacccacagctttaattgccggggcgagttcttttattgtaacacaaccagactgttcaacaataacgccaccgaggacgagacaatcaccctgccttgccggatcaagcagatcatcaatatgtggcagggagtgggaagagcaatgtacgcaccacctatcgccggcaatatcacctgtaagagcaacatcaccggactgctgctggtgagagacggaggagaggataacaagacagaggagatctttcggcccggcggcggcaatatgaaggacaactggagatccgagctgtacaagtataaagtgatcgagctgaagccactgggaatcgcacctaccggatgcaagaggagagtggtggagggaggctctggaggaggaggaagcggaggaggaggatccggcggcgccgtgggcatcggagccgtgttcctgggctttctgggagcagcaggatctaccatgggagcagcaagcctgacactgaccgtgcaggccaggcagctgctgtctagcatcgtgcagcagcagtccaatctgctgagggcaccagaggcacagcagcacatgctgcagctgacagtgtggggcatcaagcagctgcagacccgggtgctggccatcgagagatacctgaaggatcagcagctgctgggcatctggggctgctctggcaagctgatctgctgtaccaatgtgccctggaactcctcttggtccaacaagtctcagacagacatctggaataacatgacctggatggagtgggacagggagatctctaattacacagataccatctatcgcctgctggaggacagccagacccagcaggagaagaacgagaaggacctgctggccctggattga

SEQ ID NO: 4 - protein, 1349 aa, Artificial Sequence
  Synthetic construct
MPMGSLQPLATLYLLGMLVASVLAAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCTVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGGGSGGGGSGGGGSGGAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGSGGSGATNFSLLKQAGDVEENPGPGSGMPMGSLQPLATLYLLGMLVASVLAVGNMWVTVYYGVPVWTDAKTTLFCASDTKAYDREVHNVWATHACVPTDPNPQEIVLENVTENFNMWKNDMVDQMHEDIISLWDQSLKPCVKLTPLCVTLHCTNATFKNNVTNDMNKEIRNCSFNTTTEIRDKKQQGYALFYRPDIVLLKENRNNSNNSEYILINCNASTITQACPKVNFDPIPIHYCAPAGYAILKCNNKTFSGKGPCNNVSTVQCTHGIKPVVSTQLLLNGSLAEKEIIIRSENLTDNVKTIIVHLNKSVEIVCTRPNNNTRKSMRIGPGQTFYATGDIIGDIRQAYCNISGSKWNETLKRVKEKLQENYNNNKTIKFAPSSGGDLEITTHSFNCRGEFFYCNTTRLFNNNATEDETITLPCRIKQIINMWQGVGRAMYAPPIAGNITCKSNITGLLLVRDGGEDNKTEEIFRPGGGNMKDNWRSELYKYKVIELKPLGIAPTGCKRRVVEGGSGGGGSGGGGSGGAVGIGAVFLGFLGAAGSTMGAASLTLTVQARQLLSSIVQQQSNLLRAPEAQQHMLQLTVWGIKQLQTRVLAIERYLKDQQLLGIWGCSGKLICCTNVPWNSSWSNKSQTDIWNNMTWMEWDREISNYTDTIYRLLEDSQTQQEKNEKDLLALD

SEQ ID NO: 5 - DNA, 1929 bp, Artificial Sequence
  Synthetic construct
gccgaaaacctgtgggtcaccgtgtattatggagtgcccgtctggaaagatgctgaaactaccctgttctgtgcctctgatgctaaggcctacgagaccgaaaagcacaatgtctgggctactcatgcatgcgtgcccaccgacccaaacccccaggagatccacctggaaaatgtgaccgaggaattcaacatgtggaaaaacaatatggtggagcagatgcatacagacatcattagcctgtgggatcagtccctgaagccctgcgtcaaactgactcctctgtgcgtgaccctgcagtgtaccaatgtcacaaacaatatcaccgacgatatgaggggcgagctgaagaattgtagcttcaacatgaccacagaactgagagacaagaaacagaaagtgtactccctgttttataggctggatgtggtccagatcaatgagaaccaggggaatcggagcaacaattccaacaaggaatacagactgatcaattgcaacacttccgccattacccaggcttgtcctaaagtgtcttttgagcctatcccaattcattattgcgccccagctggcttcgccatcctgaagtgtaaagataagaagttcaacggaactggcccctgcccttccgtgtctacagtccagtgtactcacgggattaagcctgtggtctctacacagctgctgctgaatggaagtctggctgaggaagaagtgatgatccggagcgagaacattaccaacaatgccaagaatatcctggtccagttcaacacaccagtgcagattaattgcacaagacccaacaataacactcgaaaatctatccggattgggccaggacaggccttttacgctacaggggacatcattggagatatcagacaggctcactgtaccgtgagtaaggcaacctggaacgagacactgggcaaggtggtcaaacagctgaggaaacatttcgggaataacaccatcattcgctttgccaatagctccggaggggacctggaggtcactacccactccttcaactgcggaggcgaattcttttactgtaacacatctggcctgtttaatagtacatggatctctaacactagtgtgcagggcagtaattcaactgggtcaaacgatagcatcaccctgccatgccgaattaagcagatcattaatatgtggcagcggatcggccaggcaatgtatgccccccctatccagggggtcattcgctgcgtgagcaatatcaccggactgattctgacacgagacgggggcagcaccaactctacaactgaaacattccggcccggcgggggagacatgagagataactggaggtccgagctgtacaagtataaagtggtcaagatcgaacctctgggagtggcaccaaccagatgcaagcgaagagtggtcggaggcggcagcggcggcggcggctccggcggcggcggctctggcggcgcagtcggaattggggccgtgttcctgggatttctgggcgccgctgggagtacaatgggagcagcctcaatgactctgaccgtgcaggccaggaatctgctgagcggcatcgtccagcagcagtccaacctgctgcgcgctcctgaagcacagcagcacctgctgaagctgaccgtgtggggcatcaaacagctgcaggctagggtgctggcagtcgagcggtacctgagagaccagcagctgctgggaatctggggctgctctgggaagctgatttgttgcacaaatgtgccttggaactctagttggtcaaatcgcaacctgagcgagatctgggacaatatgacttggctgcagtgggataaagaaattagtaactacacccagatcatctacggcctgctggaagagtcacagaatcagcaggagaagaacgaacaggacctgctggcactggat

SEQ ID NO: 6 - protein, 643 aa, Artificial Sequence
  Synthetic construct
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCTVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGGGSGGGGSGGGGSGGAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 7 - DNA, 1890 bp, Artificial Sequence
  Synthetic construct
gtgggcaacatgtgggtgacagtgtactatggcgtgcccgtgtggaccgatgccaagaccacactgttctgcgcctccgacacaaaggcctacgatcgggaggtgcacaacgtgtgggcaacacacgcatgcgtgccaaccgacccaaatccccaggagatcgtgctggagaacgtgaccgagaacttcaacatgtggaagaacgacatggtggatcagatgcacgaggacatcatcagcctgtgggatcagtccctgaagccatgcgtgaagctgacacccctgtgcgtgaccctgcactgtacaaacgccacctttaagaacaatgtgaccaatgatatgaacaaggagatcaggaattgttctttcaacaccacaaccgagatccgcgataagaagcagcagggctacgccctgttttataggcctgacatcgtgctgctgaaggagaatcgcaacaattctaacaatagcgagtatatcctgatcaattgcaacgccagcacaatcacccaggcctgtcccaaggtgaacttcgaccctatcccaatccactactgcgcccctgccggctatgccatcctgaagtgtaacaacaagaccttcagcggcaagggcccatgcaacaacgtgagcacagtgcagtgtacccacggcatcaagcccgtggtgtccacccagctgctgctgaatggctctctggccgagaaggagatcatcatcaggtccgagaatctgacagataacgtgaagaccatcatcgtgcacctgaacaagtccgtggagatcgtgtgcacacgccctaacaataacaccaggaagtctatgcgcatcggcccaggccagacattctacgccaccggcgacatcatcggcgatatccggcaggcctattgtaatatcagcggctccaagtggaacgagacactgaagagagtgaaggagaagctgcaggagaactacaataacaataagaccatcaagttcgcaccaagctccggaggcgatctggagatcacaacccacagctttaattgccggggcgagttcttttattgtaacacaaccagactgttcaacaataacgccaccgaggacgagacaatcaccctgccttgccggatcaagcagatcatcaatatgtggcagggagtgggaagagcaatgtacgcaccacctatcgccggcaatatcacctgtaagagcaacatcaccggactgctgctggtgagagacggaggagaggataacaagacagaggagatctttcggcccggcggcggcaatatgaaggacaactggagatccgagctgtacaagtataaagtgatcgagctgaagccactgggaatcgcacctaccggatgcaagaggagagtggtggagggaggctctggaggaggaggaagcggaggaggaggatccggcggcgccgtgggcatcggagccgtgttcctgggctttctgggagcagcaggatctaccatgggagcagcaagcctgacactgaccgtgcaggccaggcagctgctgtctagcatcgtgcagcagcagtccaatctgctgagggcaccagaggcacagcagcacatgctgcagctgacagtgtggggcatcaagcagctgcagacccgggtgctggccatcgagagatacctgaaggatcagcagctgctgggcatctggggctgctctggcaagctgatctgctgtaccaatgtgccctggaactcctcttggtccaacaagtctcagacagacatctggaataacatgacctggatggagtgggacagggagatctctaattacacagataccatctatcgcctgctggaggacagccagacccagcaggagaagaacgagaaggacctgctggccctggat

SEQ ID NO: 8 - protein, 631 aa, Artificial Sequence
  Synthetic construct
AVGNMWVTVYYGVPVWTDAKTTLFCASDTKAYDREVHNVWATHACVPTDPNPQEIVLENVTENFNMWKNDMVDQMHEDIISLWDQSLKPCVKLTPLCVTLHCTNATFKNNVTNDMNKEIRNCSFNTTTEIRDKKQQGYALFYRPDIVLLKENRNNSNNSEYILINCNASTITQACPKVNFDPIPIHYCAPAGYAILKCNNKTFSGKGPCNNVSTVQCTHGIKPVVSTQLLLNGSLAEKEIIIRSENLTDNVKTIIVHLNKSVEIVCTRPNNNTRKSMRIGPGQTFYATGDIIGDIRQAYCNISGSKWNETLKRVKEKLQENYNNNKTIKFAPSSGGDLEITTHSFNCRGEFFYCNTTRLFNNNATEDETITLPCRIKQIINMWQGVGRAMYAPPIAGNITCKSNITGLLLVRDGGEDNKTEEIFRPGGGNMKDNWRSELYKYKVIELKPLGIAPTGCKRRVVEGGSGGGGSGGGGSGGAVGIGAVFLGFLGAAGSTMGAASLTLTVQARQLLSSIVQQQSNLLRAPEAQQHMLQLTVWGIKQLQTRVLAIERYLKDQQLLGIWGCSGKLICCTNVPWNSSWSNKSQTDIWNNMTWMEWDREISNYTDTIYRLLEDSQTQQEKNEKDLLALD

SEQ ID NO: 9 - DNA, 489 bp, Artificial Sequence
  Synthetic construct
gatatcatcaagctgctgaacgagcaagtgaataaggagatgcagagctccaacctgtacatgagcatgtctagctggtgctatacccactccctggacggagcaggactgttcctgtttgatcacgccgccgaggagtatgagcacgccaagaagctgatcatctttctgaatgagaacaatgtgcccgtgcagctgacctccatctctgcccctgagcacaagttcgagggcctgacacagatctttcagaaggcctacgagcacgagcagcacatcagcgagtccatcaacaatatcgtggaccacgccatcaagtccaaggatcacgccacattcaactttctgcagtggtacgtggccgagcagcacgaggaggaggtgctgttcaaggacatcctggataagatcgagctgatcggcaacgagaatcacggcctgtacctggccgaccagtatgtgaagggcatcgccaagtctcggaagagc

SEQ ID NO: 10 - protein, 163 aa, Artificial Sequence
  Synthetic construct
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 11 - DNA, 75 bp, Artificial Sequence
  Synthetic construct
ggaagcggagctactaacttcagcctgctgaagcaggctggagacgtggaggagaaccctggacctggaagcgga

SEQ ID NO: 12 - DNA, 75 bp, Artificial Sequence
  Synthetic construct
ggcagcggcgctactaacttcagcctgctgaagcaggctggagacgtggaggagaaccctggacctggaagcgga

SEQ ID NO: 13 - protein, 25 aa, Artificial Sequence
  Synthetic construct
GSGATNFSLLKQAGDVEENPGPGSG

SEQ ID NO: 14 - protein, 28 aa, Artificial Sequence
  Synthetic construct
GSGGSGATNFSLLKQAGDVEENPGPGSG

SEQ ID NO: 15 - DNA, 72 bp, Artificial Sequence
  Synthetic construct
atgcccatgggcagcctgcagcccctggccaccctgtacctgctgggcatgctggtggctagcgtgctggcc

SEQ ID NO: 16 - protein, 24 aa, Artificial Sequence
  Synthetic construct
MPMGSLQPLATLYLLGMLVASVLA

SEQ ID NO: 17 - DNA, 24 bp, Artificial Sequence
  Synthetic construct
gcggttggtatcggtgcggttttc

SEQ ID NO: 18 - DNA, 25 bp, Artificial Sequence
  Synthetic construct
cgcggttggtctcggtgcggttttc

SEQ ID NO: 19 - DNA, 24 bp, Artificial Sequence
  Synthetic construct
gcggttggtctcggtgcgatgatc

SEQ ID NO: 20 - protein, 8 aa, Artificial Sequence
  Synthetic construct
AVGIGAVF

SEQ ID NO: 21 - protein, 8 aa, Artificial Sequence
  Synthetic construct
AVGLGAVF

SEQ ID NO: 22 - protein, 8 aa, Artificial Sequence
  Synthetic construct
AVGLGAMI

SEQ ID NO: 23 - protein, 8 aa, Artificial Sequence
  Synthetic construct
AVGIGAMI

SEQ ID NO: 24 - protein, 8 aa, Artificial Sequence
  Synthetic construct
AVGLGAVL

SEQ ID NO: 25 - DNA, 9 bp, Artificial Sequence
  Synthetic construct
ggaagcgga

SEQ ID NO: 26 - DNA, 6 bp, Artificial Sequence
  Synthetic construct
agcgga

SEQ ID NO: 27 - DNA, 6 bp, Artificial Sequence
  Synthetic construct
agcgga

SEQ ID NO: 28 - DNA, 9 bp, Artificial Sequence
  Synthetic construct
ggcagcggc

SEQ ID NO: 29 - protein, 3 aa, Artificial Sequence
  Synthetic construct
GSG

SEQ ID NO: 30 - protein, 6 aa, Artificial Sequence
  Synthetic construct
GSGGSG

SEQ ID NO: 31 - protein, 837 aa, Artificial Sequence
  Synthetic construct
MKVMGIPKNWPRWWMWGILGLWMLLICNGEDLWVTVYYGVPVWKEANPTLFCASDAKAYKTEMHNVWATHACVPTDPNPQEMVLENVTEDFNMWKNGMVEQMHQDIISLWDQSLKPCVKLTPLCVTLNCTEVTRSSNGTINNNSTEMKNCSFNVTTDLRDKKKKEHALFYRLDIVPLDETNGTSSEYRLINCNTSTITQACPKVSFDPIPIHYCAPAGYAILKCKDKKFNGTGPCKNVSTVQCTHGIKPVVSTQLLLNGSIAEGEIIIRSENLTNNAKIIIVQLNVTVEINCTRPNNNTRRSIRIGPGQTFYATGEIIGDIRQAHCNISREKWNRTLQKVEKKLEELFPNKTIHFTSSSGGDLEITTHSFNCMGEFFYCNTSALFNNNNDSTNSNITLPCRIRQFINMWQEVGRAMYAPPIQGVITCKSNVTGLLLTRDGGIINDTEIFRPGGGDMRDNWRSELYKYKVVEIKPLGIAPTTAKRRVVEREKRAVGIGAVFLGFLGAAGSTMGAASITLTAQARQLLSGIVQQQSNLLRAIEAQQHMLQLTVWGIKQLQTRVLAIERYLKDQQLLGIWGCSGKLICTTAVPWNGSWSNKSQDEIWHNMTWMQWDKEINNYTNIIYGLLEVSQNQQEKNEQDLLALDKWQNLWSWFNITNWLWYIKIFIMIVGGLIGLRIIFTVLSIVNRVRQGYSPLSFQTLIPNPRGPDRPRGIEEEGGEQDRSRSIRLVSGFLALAWDDLRSLCLFSYHRLRDLILIAARVVELLGQRGWEALKYLGSLVQYWGLELKKSAISLFDTIAIAVAEGTDRIIEVLQGIGRAICNIPRRIRQGFEAALQ

SEQ ID NO: 32 - protein, 652 aa, Artificial Sequence
  Synthetic construct
MPMGSLQPLATLYLLGMLVASVLAGEDLWVTVYYGVPVWKEANPTLFCASDAKAYKTEMHNVWATHACVPTDPNPQEMVLENVTEDFNMWKNGMVEQMHQDIISLWDQSLKPCVKLTPLCVTLNCTEVTRSSNGTINNNSTEMKNCSFNVTTDLRDKKKKEHALFYRLDIVPLDETNGTSSEYRLINCNTSTCTQACPKVSFDPIPIHYCAPAGYAILKCKDKKFNGTGPCKNVSTVQCTHGIKPVVSTQLLLNGSIAEGEIIIRSENLTNNAKIIIVQLNVTVEINCTRPNNNTRRSIRIGPGQTFYATGEIIGDIRQAHCNISREKWNRTLQKVEKKLEELFPNKTIHFTSSSGGDLEITTHSFNCMGEFFYCNTSALFNNNNDSTNSNITLPCRIRQFINMWQEVGRCMYAPPIQGVITCKSNVTGLLLTRDGGIINDTEIFRPGGGDMRDNWRSELYKYKVVEIKPLGIAPTTCKRRVVEGGSGGGGSGGGGSGGAVGIGAVFLGFLGAAGSTMGAASITLTAQARQLLSGIVQQQSNLLRAPEAQQHMLQLTVWGIKQLQTRVLAIERYLKDQQLLGIWGCSGKLICCTAVPWNGSWSNKSQDEIWHNMTWMQWDKEINNYTNIIYGLLEVSQNQQEKNEQDLLALD

SEQ ID NO: 33 - protein, 844 aa, Artificial Sequence
  Synthetic construct
MPMGSLQPLATLYLLGMLVASVLAGEDLWVTVYYGVPVWKEANPTLFCASDAKAYKTEMHNVWATHACVPTDPNPQEMVLENVTEDFNMWKNGMVEQMHQDIISLWDQSLKPCVKLTPLCVTLNCTEVTRSSNGTINNNSTEMKNCSFNVTTDLRDKKKKEHALFYRLDIVPLDETNGTSSEYRLINCNTSTCTQACPKVSFDPIPIHYCAPAGYAILKCKDKKFNGTGPCKNVSTVQCTHGIKPVVSTQLLLNGSIAEGEIIIRSENLTNNAKIIIVQLNVTVEINCTRPNNNTRRSIRIGPGQTFYATGEIIGDIRQAHCNISREKWNRTLQKVEKKLEELFPNKTIHFTSSSGGDLEITTHSFNCMGEFFYCNTSALFNNNNDSTNSNITLPCRIRQFINMWQEVGRCMYAPPIQGVITCKSNVTGLLLTRDGGIINDTEIFRPGGGDMRDNWRSELYKYKVVEIKPLGIAPTTCKRRVVEGGSGGGGSGGGGSGGAVGIGAVFLGFLGAAGSTMGAASITLTAQARQLLSGIVQQQSNLLRAPEAQQHMLQLTVWGIKQLQTRVLAIERYLKDQQLLGIWGCSGKLICCTAVPWNGSWSNKSQDEIWHNMTWMQWDKEINNYTNIIYGLLEVSQNQQEKNEQDLLALDKWQNLWSWFNITNWLWYIKIFIMIVGGLIGLRIIFTVLSIVNRVRQGYSPLSFQTLIPNPRGPDRPRGIEEEGGEQDRSRSIRLVSGFLALAWDDLRSLCLFSYHRLRDLILIAARVVELLGQRGWEALKYLGSLVQYWGLELKKSAISLFDTIAIAVAEGTDRIIEVLQGIGRAICNIPRRIRQGFEAALQ

SEQ ID NO: 34 - protein, 830 aa, Artificial Sequence
  Synthetic construct
MPMGSLQPLATLYLLGMLVASVLAGEDLWVTVYYGVPVWKEANPTLFCASDAKAYKTEMHNVWATHACVPTDPNPQEMVLENVTEDFNMWKNGMVEQMHQDIISLWDQSLKPCVKLTPLCVTLNCTEVTRSSNGTINNNSTEMKNCSFNVTTDLRDKKKKEHALFYRLDIVPLDETNGTSSEYRLINCNTSTCTQACPKVSFDPIPIHYCAPAGYAILKCKDKKFNGTGPCKNVSTVQCTHGIKPVVSTQLLLNGSIAEGEIIIRSENLTNNAKIIIVQLNVTVEINCTRPNNNTRRSIRIGPGQTFYATGEIIGDIRQAHCNISREKWNRTLQKVEKKLEELFPNKTIHFTSSSGGDLEITTHSFNCMGEFFYCNTSALFNNNNDSTNSNITLPCRIRQFINMWQEVGRCMYAPPIQGVITCKSNVTGLLLTRDGGIINDTEIFRPGGGDMRDNWRSELYKYKVVEIKPLGIAPTTCKRRVVEGGSGGGGSGGGGSGGAVGIGAVFLGFLGAAGSTMGAASITLTAQARQLLSGIVQQQSNLLRAPEAQQHMLQLTVWGIKQLQTRVLAIERYLKDQQLLGIWGCSGKLICCTAVPWNGSWSNKSQDEIWHNMTWMQWDKEINNYTNIIYGLLEVSQNQQEKNEQDLLALDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 35 - protein, 862 aa, Artificial Sequence
  Synthetic construct
MRVKGIKKNYQHWWRWGMMIFGLLMICSAADKLWVTVYYGVPVWKETTTTLFCASDARAYDTEVHNVWATHACVPTDPNPQEVVLGNVTENFNMWKNNMVEQMHEDIISLWDQSLKPCVRLTPLCVTLNCIDYYGNTTNSNNSSETMMEKGEIKNCSFNITTRLKDKMQKEYALFYKYDIVPIDNRVGNDTSNATSYRLTSCNTSVITQACPKVSFEPIPIHYCAPAGFAILKCNDKKFNGTGPCKNVSTVQCTHGIKPVVSTQLLLNGSLAEEEVMIRSENFTDNAKTIIVQLNETVEINCTRPNNNTRKSIHMGPGKVFYTTGEIIGDIRQAHCNINRAKWNNTLIKIVEKLRVKFNKTISFKQSSGGDPEIEMHSFNCGGEFFYCNTTQLFNSTWFNNATLNVNSNVTEGSENITLPCRIRQIVNMWQEVGKAMYAPPIQGQIRCSSNITGLLLTRDGGGSNSSNTSEEVFRPGGGNMRDNWRSELYKYKVVKIEPLGIAPTKAKRRVVQREKRTVGIGALFLGFLGAAGSTMGAASMTLTVQARQLLSGIVQQQNNLLRAIQAQQHLLQLTVWGIKQLQARVLAVERYLKDQQLLGIWGCSGKLICTTAVPWNASWSNKSLNEIWDNMTWMEWEKEIDNYTSLIYTLIEESQNQQEKNEQELLELDKWASLWNWFSITNWLWYIKIFIMIVGGSIGLRIVFAVLSIVNRVRQGYSPLSFQTRLPTPRGPDRPEGIEEEGGERDRDRSGQLVNGFLAIIWVDLRSLCLFSYHRLRDLLLIVARVVELLGRRGWEALNYWWNLLQYWSQELKKSAISLLNATAIAVAEGTDRVIEVVQRTCRAIIHIPRRIRQGLERLLL

SEQ ID NO: 36 - protein, 676 aa, Artificial Sequence
  Synthetic construct
MPMGSLQPLATLYLLGMLVASVLAADKLWVTVYYGVPVWKETTTTLFCASDARAYDTEVHNVWATHACVPTDPNPQEVVLGNVTENFNMWKNNMVEQMHEDIISLWDQSLKPCVRLTPLCVTLNCIDYYGNTTNSNNSSETMMEKGEIKNCSFNITTRLKDKMQKEYALFYKYDIVPIDNRVGNDTSNATSYRLTSCNTSVCTQACPKVSFEPIPIHYCAPAGFAILKCNDKKFNGTGPCKNVSTVQCTHGIKPVVSTQLLLNGSLAEEEVMIRSENFTDNAKTIIVQLNETVEINCTRPNNNTRKSIHMGPGKVFYTTGEIIGDIRQAHCNINRAKWNNTLIKIVEKLRVKFNKTISFKQSSGGDPEIEMHSFNCGGEFFYCNTTQLFNSTWFNNATLNVNSNVTEGSENITLPCRIRQIVNMWQEVGKCMYAPPIQGQIRCSSNITGLLLTRDGGGSNSSNTSEEVFRPGGGNMRDNWRSELYKYKVVKIEPLGIAPTKCKRRVVQGGSGGGGSGGGGSGGTVGIGALFLGFLGAAGSTMGAASMTLTVQARQLLSGIVQQQNNLLRAPQAQQHLLQLTVWGIKQLQARVLAVERYLKDQQLLGIWGCSGKLICCTAVPWNASWSNKSLNEIWDNMTWMEWEKEIDNYTSLIYTLIEESQNQQEKNEQELLELD

SEQ ID NO: 37 - protein, 868 aa, Artificial Sequence
  Synthetic construct
MPMGSLQPLATLYLLGMLVASVLAADKLWVTVYYGVPVWKETTTTLFCASDARAYDTEVHNVWATHACVPTDPNPQEVVLGNVTENFNMWKNNMVEQMHEDIISLWDQSLKPCVRLTPLCVTLNCIDYYGNTTNSNNSSETMMEKGEIKNCSFNITTRLKDKMQKEYALFYKYDIVPIDNRVGNDTSNATSYRLTSCNTSVCTQACPKVSFEPIPIHYCAPAGFAILKCNDKKFNGTGPCKNVSTVQCTHGIKPVVSTQLLLNGSLAEEEVMIRSENFTDNAKTIIVQLNETVEINCTRPNNNTRKSIHMGPGKVFYTTGEIIGDIRQAHCNINRAKWNNTLIKIVEKLRVKFNKTISFKQSSGGDPEIEMHSFNCGGEFFYCNTTQLFNSTWFNNATLNVNSNVTEGSENITLPCRIRQIVNMWQEVGKCMYAPPIQGQIRCSSNITGLLLTRDGGGSNSSNTSEEVFRPGGGNMRDNWRSELYKYKVVKIEPLGIAPTKCKRRVVQGGSGGGGSGGGGSGGTVGIGALFLGFLGAAGSTMGAASMTLTVQARQLLSGIVQQQNNLLRAPQAQQHLLQLTVWGIKQLQARVLAVERYLKDQQLLGIWGCSGKLICCTAVPWNASWSNKSLNEIWDNMTWMEWEKEIDNYTSLIYTLIEESQNQQEKNEQELLELDKWASLWNWFSITNWLWYIKIFIMIVGGSIGLRIVFAVLSIVNRVRQGYSPLSFQTRLPTPRGPDRPEGIEEEGGERDRDRSGQLVNGFLAIIWVDLRSLCLFSYHRLRDLLLIVARVVELLGRRGWEALNYWWNLLQYWSQELKKSAISLLNATAIAVAEGTDRVIEVVQRTCRAIIHIPRRIRQGLERLLL

SEQ ID NO: 38 - protein, 854 aa, Artificial Sequence
  Synthetic construct
MPMGSLQPLATLYLLGMLVASVLAADKLWVTVYYGVPVWKETTTTLFCASDARAYDTEVHNVWATHACVPTDPNPQEVVLGNVTENFNMWKNNMVEQMHEDIISLWDQSLKPCVRLTPLCVTLNCIDYYGNTTNSNNSSETMMEKGEIKNCSFNITTRLKDKMQKEYALFYKYDIVPIDNRVGNDTSNATSYRLTSCNTSVCTQACPKVSFEPIPIHYCAPAGFAILKCNDKKFNGTGPCKNVSTVQCTHGIKPVVSTQLLLNGSLAEEEVMIRSENFTDNAKTIIVQLNETVEINCTRPNNNTRKSIHMGPGKVFYTTGEIIGDIRQAHCNINRAKWNNTLIKIVEKLRVKFNKTISFKQSSGGDPEIEMHSFNCGGEFFYCNTTQLFNSTWFNNATLNVNSNVTEGSENITLPCRIRQIVNMWQEVGKCMYAPPIQGQIRCSSNITGLLLTRDGGGSNSSNTSEEVFRPGGGNMRDNWRSELYKYKVVKIEPLGIAPTKCKRRVVQGGSGGGGSGGGGSGGTVGIGALFLGFLGAAGSTMGAASMTLTVQARQLLSGIVQQQNNLLRAPQAQQHLLQLTVWGIKQLQARVLAVERYLKDQQLLGIWGCSGKLICCTAVPWNASWSNKSLNEIWDNMTWMEWEKEIDNYTSLIYTLIEESQNQQEKNEQELLELDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 39 - protein, 860 aa, Artificial Sequence
  Synthetic construct
MRVMGILRSYQQWWIWGILGFWMLMICNVWGNLWVTVYYGVPVWKEAKTTLFCASDAKAHKEEVHNIWATHACVPTDPNPQEIVLKNVTENFNMWKNDMVDQMHEDIISLWDQSLKPCVKLTPLCVTLNCSDVKIKGTNATYNNATYNNNNTISDMKNCSFNTTTEITDKKKKEYALFYKLDVVALDGKETNSTNSSEYRLINCNTSAVTQACPKVSFDPIPIHYCAPAGYAILKCNNKTFNGTGPCNNVSTVQCTHGIKPVVSTQLLLNGSLAEEEVVIRFENLTNNAKIIIVHLNESVEINCTRPSNNTRKSVRIGPGQTFFATGDIIGDIRQAHCNISRKKWNTTLQRVKEKLKEKFPNKTIQFAPSSGGDLEITTHSFNCRGEFFYCYTSDLFNSTYMSNNTGGANITLQCRIKQIIRMWQGVGQAMYAPPIAGNITCKSNITGLLLTRDGGKEKNDTETFRPGGGDMRDNWRSELYKYKVVEIKPLGIAPDKAKRRVVEREKRAVGIGAVFLGFLGAAGSTMGAASMTLTVQARQLLSGIVQQQSNLLRAIEAQQHMLQLTVWGIKQLQTRVLAIERYLKDQQLLGIWGCSGKLICTTAVPWNASWSNKSYEEIWGNMTWMQWDREINNYTNTIYSLLEESQNQQEKNEKDLLALDSWESLWSWFNITNWLWYIRIFIIIVGGLIGLRIIFAVLSIVNRVRQGYSPLSFQTLTPSPREPDRLGRIEEEGGEQDRARSVRLVNGFLALAWEDLRSLCLFSYHRLRDLILIAARAAALLGRSSLWGLQKGWEALKYLGSLVQYWGLELKKSAISLFDAIAITVAEGTDRIINIVQRISRAFYNIPRRIRQGFEATLQ

SEQ ID NO: 40 - protein, 659 aa, Artificial Sequence
  Synthetic construct
MKAKLLVLLCTFTATYAGNLWVTVYYGVPVWKEAKTTLFCASDAKAHKEEVHNIWATHACVPTDPNPQEIVLKNVTENFNMWKNDMVDQMHEDIISLWDQSLKPCVKLTPLCVTLNCSDVKIKGTNATYNNATYNNNNTISDMKNCSFNTTTEITDKKKKEYALFYKLDVVALDGKETNSTNSSEYRLINCNTSACTQACPKVSFDPIPIHYCAPAGYAILKCNNKTFNGTGPCNNVSTVQCTHGIKPVVSTQLLLNGSLAEEEVVIRFENLTNNAKIIIVHLNESVEINCTRPSNNTRKSVRIGPGQTFFATGDIIGDIRQAHCNISRKKWNTTLQRVKEKLKEKFPNKTIQFAPSSGGDLEITTHSFNCRGEFFYCYTSDLFNSTYMSNNTGGANITLQCRIKQIIRMWQGVGQCMYAPPIAGNITCKSNITGLLLTRDGGKEKNDTETFRPGGGDMRDNWRSELYKYKVVEIKPLGIAPDKCKRRVVEGGSGGGGSGGGGSGGAVGIGAVFLGFLGAAGSTMGAASMTLTVQARQLLSGIVQQQSNLLRAPEAQQHMLQLTVWGIKQLQTRVLAIERYLKDQQLLGIWGCSGKLICCTAVPWNASWSNKSYEEIWGNMTWMQWDREINNYTNTIYSLLEESQNQQEKNEKDLLALD

SEQ ID NO: 41 - protein, 858 aa, Artificial Sequence
  Synthetic construct
MKAKLLVLLCTFTATYAGNLWVTVYYGVPVWKEAKTTLFCASDAKAHKEEVHNIWATHACVPTDPNPQEIVLKNVTENFNMWKNDMVDQMHEDIISLWDQSLKPCVKLTPLCVTLNCSDVKIKGTNATYNNATYNNNNTISDMKNCSFNTTTEITDKKKKEYALFYKLDVVALDGKETNSTNSSEYRLINCNTSACTQACPKVSFDPIPIHYCAPAGYAILKCNNKTFNGTGPCNNVSTVQCTHGIKPVVSTQLLLNGSLAEEEVVIRFENLTNNAKIIIVHLNESVEINCTRPSNNTRKSVRIGPGQTFFATGDIIGDIRQAHCNISRKKWNTTLQRVKEKLKEKFPNKTIQFAPSSGGDLEITTHSFNCRGEFFYCYTSDLFNSTYMSNNTGGANITLQCRIKQIIRMWQGVGQCMYAPPIAGNITCKSNITGLLLTRDGGKEKNDTETFRPGGGDMRDNWRSELYKYKVVEIKPLGIAPDKCKRRVVEGGSGGGGSGGGGSGGAVGIGAVFLGFLGAAGSTMGAASMTLTVQARQLLSGIVQQQSNLLRAPEAQQHMLQLTVWGIKQLQTRVLAIERYLKDQQLLGIWGCSGKLICCTAVPWNASWSNKSYEEIWGNMTWMQWDREINNYTNTIYSLLEESQNQQEKNEKDLLALDSWESLWSWFNITNWLWYIRIFIIIVGGLIGLRIIFAVLSIVNRVRQGYSPLSFQTLTPSPREPDRLGRIEEEGGEQDRARSVRLVNGFLALAWEDLRSLCLFSYHRLRDLILIAARAAALLGRSSLWGLQKGWEALKYLGSLVQYWGLELKKSAISLFDAIAITVAEGTDRIINIVQRISRAFYNIPRRIRQGFEATLQ

SEQ ID NO: 42 - protein, 847 aa, Artificial Sequence
  Synthetic construct
MKAKLLVLLCTFTATYAGNLWVTVYYGVPVWKEAKTTLFCASDAKAHKEEVHNIWATHACVPTDPNPQEIVLKNVTENFNMWKNDMVDQMHEDIISLWDQSLKPCVKLTPLCVTLNCSDVKIKGTNATYNNATYNNNNTISDMKNCSFNTTTEITDKKKKEYALFYKLDVVALDGKETNSTNSSEYRLINCNTSACTQACPKVSFDPIPIHYCAPAGYAILKCNNKTFNGTGPCNNVSTVQCTHGIKPVVSTQLLLNGSLAEEEVVIRFENLTNNAKIIIVHLNESVEINCTRPSNNTRKSVRIGPGQTFFATGDIIGDIRQAHCNISRKKWNTTLQRVKEKLKEKFPNKTIQFAPSSGGDLEITTHSFNCRGEFFYCYTSDLFNSTYMSNNTGGANITLQCRIKQIIRMWQGVGQCMYAPPIAGNITCKSNITGLLLTRDGGKEKNDTETFRPGGGDMRDNWRSELYKYKVVEIKPLGIAPDKCKRRVVEGGSGGGGSGGGGSGGAVGIGAVFLGFLGAAGSTMGAASMTLTVQARQLLSGIVQQQSNLLRAPEAQQHMLQLTVWGIKQLQTRVLAIERYLKDQQLLGIWGCSGKLICCTAVPWNASWSNKSYEEIWGNMTWMQWDREINNYTNTIYSLLEESQNQQEKNEKDLLALDGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 43 - protein, 876 aa, Artificial Sequence
  Synthetic construct
MRVKEKYQHLWRWGWRWGTMLLGMLMICSATEKLWVTVYYGVPVWKEATTTLFCASDAKAYETEVHNVWATHACVPTDPNPQEVLLENVTENFNMWKNNMVEQMQEDIISLWDQSLKPCVKLTPLCVTLECHDVNVNGTANNGTTNVTESGVNSSDVTSNNVTNSNWGTMEKGEIKNCSFNITTNIRDKMQKETAQFYKLDIVPIEDQNKTNNTLYRLINCNTSVITQACPKVSFEPIPIHYCTPAGFAILKCNDRNFNGTGPCKNVSTVQCTHGIKPVVSTQLLLNGSLAEAEVVIRSENFTNNAKTIIIQLNETVEINCTRPNNNTSKRISIGPGRAFRATKIIGNIRQAHCNISRATWNSTLKKIVAKLREQFGNKTIVFQPSSGGDPEIVMHSFNCGGEFFYCNTTQLFNSTWNSTEESNSTEEGTITLPCRIKQIINMWQEVGKAMYAPPIEGQIRCSSNITGLLLTRDGGNNNKTNGTEIFRPGGGDMRDNWRSELYKYKVVKIEPLGVAPTKAKRRVVQREKRAVGIVGAMFLGFLGAAGSTMGAASMTLTVQARLLLSGIVQQQNNLLRAIEAQQHLLQLTVWGIKQLQARVLAVERYLKDQQLLGIWGCSGKLICTTTVPWNTSWSNKSLSEIWDNMTWMQWEREIDNYTSLIYTLIEESQNQQEKNEQELLELDKWAGLWNWFEITNWLWYIKIFIMIVGGLVGLRIVFAVLSIVNRVRQGYSPVSFQTHLPAPRGPDRPEGIEEEGGERDRGRSVRLVNGFLALIWDDLRSLCLFSYHRLRDLLLIIARIVELLGRRGWEALKYWWNLLQYWSQELKNSAVNLLDATAIAVAEGTDRIIEVVRRAFRAILHIPTRIRQGLERALL

SEQ ID NO: 44 - protein, 689 aa, Artificial Sequence
  Synthetic construct
MPMGSLQPLATLYLLGMLVASVLATEKLWVTVYYGVPVWKEATTTLFCASDAKAYETEVHNVWATHACVPTDPNPQEVLLENVTENFNMWKNNMVEQMQEDIISLWDQSLKPCVKLTPLCVTLECHDVNVNGTANNGTTNVTESGVNSSDVTSNNVTNSNWGTMEKGEIKNCSFNITTNIRDKMQKETAQFYKLDIVPIEDQNKTNNTLYRLINCNTSVCTQACPKVSFEPIPIHYCTPAGFAILKCNDRNFNGTGPCKNVSTVQCTHGIKPVVSTQLLLNGSLAEAEVVIRSENFTNNAKTIIIQLNETVEINCTRPNNNTSKRISIGPGRAFRATKIIGNIRQAHCNISRATWNSTLKKIVAKLREQFGNKTIVFQPSSGGDPEIVMHSFNCGGEFFYCNTTQLFNSTWNSTEESNSTEEGTITLPCRIKQIINMWQEVGKCMYAPPIEGQIRCSSNITGLLLTRDGGNNNKTNGTEIFRPGGGDMRDNWRSELYKYKVVKIEPLGVAPTKCKRRVVQGGSGGGGSGGGGSGGAVGIVGAMFLGFLGAAGSTMGAASMTLTVQARLLLSGIVQQQNNLLRAPEAQQHLLQLTVWGIKQLQARVLAVERYLKDQQLLGIWGCSGKLICCTTVPWNTSWSNKSLSEIWDNMTWMQWEREIDNYTSLIYTLIEESQNQQEKNEQELLELD

SEQ ID NO: 45 - protein, 881 aa, Artificial Sequence
  Synthetic construct
MPMGSLQPLATLYLLGMLVASVLATEKLWVTVYYGVPVWKEATTTLFCASDAKAYETEVHNVWATHACVPTDPNPQEVLLENVTENFNMWKNNMVEQMQEDIISLWDQSLKPCVKLTPLCVTLECHDVNVNGTANNGTTNVTESGVNSSDVTSNNVTNSNWGTMEKGEIKNCSFNITTNIRDKMQKETAQFYKLDIVPIEDQNKTNNTLYRLINCNTSVCTQACPKVSFEPIPIHYCTPAGFAILKCNDRNFNGTGPCKNVSTVQCTHGIKPVVSTQLLLNGSLAEAEVVIRSENFTNNAKTIIIQLNETVEINCTRPNNNTSKRISIGPGRAFRATKIIGNIRQAHCNISRATWNSTLKKIVAKLREQFGNKTIVFQPSSGGDPEIVMHSFNCGGEFFYCNTTQLFNSTWNSTEESNSTEEGTITLPCRIKQIINMWQEVGKCMYAPPIEGQIRCSSNITGLLLTRDGGNNNKTNGTEIFRPGGGDMRDNWRSELYKYKVVKIEPLGVAPTKCKRRVVQGGSGGGGSGGGGSGGAVGIVGAMFLGFLGAAGSTMGAASMTLTVQARLLLSGIVQQQNNLLRAPEAQQHLLQLTVWGIKQLQARVLAVERYLKDQQLLGIWGCSGKLICCTTVPWNTSWSNKSLSEIWDNMTWMQWEREIDNYTSLIYTLIEESQNQQEKNEQELLELDKWAGLWNWFEITNWLWYIKIFIMIVGGLVGLRIVFAVLSIVNRVRQGYSPVSFQTHLPAPRGPDRPEGIEEEGGERDRGRSVRLVNGFLALIWDDLRSLCLFSYHRLRDLLLIIARIVELLGRRGWEALKYWWNLLQYWSQELKNSAVNLLDATAIAVAEGTDRIIEVVRRAFRAILHIPTRIRQGLERALL

SEQ ID NO: 46 - protein, 877 aa, Artificial Sequence
  Synthetic construct
MPMGSLQPLATLYLLGMLVASVLATEKLWVTVYYGVPVWKEATTTLFCASDAKAYETEVHNVWATHACVPTDPNPQEVLLENVTENFNMWKNNMVEQMQEDIISLWDQSLKPCVKLTPLCVTLECHDVNVNGTANNGTTNVTESGVNSSDVTSNNVTNSNWGTMEKGEIKNCSFNITTNIRDKMQKETAQFYKLDIVPIEDQNKTNNTLYRLINCNTSVCTQACPKVSFEPIPIHYCTPAGFAILKCNDRNFNGTGPCKNVSTVQCTHGIKPVVSTQLLLNGSLAEAEVVIRSENFTNNAKTIIIQLNETVEINCTRPNNNTSKRISIGPGRAFRATKIIGNIRQAHCNISRATWNSTLKKIVAKLREQFGNKTIVFQPSSGGDPEIVMHSFNCGGEFFYCNTTQLFNSTWNSTEESNSTEEGTITLPCRIKQIINMWQEVGKCMYAPPIEGQIRCSSNITGLLLTRDGGNNNKTNGTEIFRPGGGDMRDNWRSELYKYKVVKIEPLGVAPTKCKRRVVQGGSGGGGSGGGGSGGAVGIVGAMFLGFLGAAGSTMGAASMTLTVQARLLLSGIVQQQNNLLRAPEAQQHLLQLTVWGIKQLQARVLAVERYLKDQQLLGIWGCSGKLICCTTVPWNTSWSNKSLSEIWDNMTWMQWEREIDNYTSLIYTLIEESQNQQEKNEQELLELDGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 47 - protein, 860 aa, Artificial Sequence
  Synthetic construct
MRVMGIQRNCQNLLTWGTMILGIIIFCSATDNLWVTVYYGVPVWKDAETTLFCASDAKAYATEKHNVWATHACVPTDPNPQEIHLENVTEEFNMWKNNMVEQMHTDIISLWDQSLKPCVKLTPLCVTLSCSNAKVSYSNATVNNTIQDEIKNCSFNTTTVLRDKRQKVYSLFYRLDIVQIDNSSSDSSSSEYRLINCNTSAITQACPKVTFEPIPIHYCAPAGFAILKCKDEEFNGTGPCKNVSTVQCTHGIKPVVSTQLLLNGSLAKREVKIRSENITNNAKNIIVQFVDPVEINCTRPNNNTRKSIHIGPGQAFYATGDIIGDIRQAHCNVSRSSWNKTLQQVAKQLGTYFKNKTIVFNTSSGGDPEITTHSFNCAGEFFYCDTSGLFNSSWNDTTWKESNSTGSNDTITLLCRIKQIINMWQRTGQAMYAPPIPGLISCKSNITGIILTRDGGNSHRTEETFRPGGGDMRDNWRSELYRYKVVQIEPLGVAPTRARRRVVQREKRAVGIGAVFLGFLGAAGSTMGAASITLTVQARQLLSGIVQQQSNLLRAIEAQQHLLKLTVWGIKQLQARVLAVERYLRDQQLLGIWGCSGKLICTTNVPWNSSWSNKSYNDIWDNMTWLQWDKEIHNYTQLIYNLIEESQNQQEKNEQDLLALDKWANLWNWFNITNWLWYIKIFIMVVGGLIGLRIVFAVLSIINRVRQGYSPLSFQTHLPNPRDLDRPERIEEEGGEQGRDRSIRLVSGFLALAWDDLRSLCLFSYHRLRDFILIAARTVELLGQSSLKGLRLGWESLKYLWNLLGYWVRELKISAVNLVDTIAIAVAGWTDRVIEIGQRIGRAIRHIPRRIRQGLERALL

SEQ ID NO: 48 - protein, 667 aa, Artificial Sequence
  Synthetic construct
MPMGSLQPLATLYLLGMLVASVLATDNLWVTVYYGVPVWKDAETTLFCASDAKAYATEKHNVWATHACVPTDPNPQEIHLENVTEEFNMWKNNMVEQMHTDIISLWDQSLKPCVKLTPLCVTLSCSNAKVSYSNATVNNTIQDEIKNCSFNTTTVLRDKRQKVYSLFYRLDIVQIDNSSSDSSSSEYRLINCNTSACTQACPKVTFEPIPIHYCAPAGFAILKCKDEEFNGTGPCKNVSTVQCTHGIKPVVSTQLLLNGSLAKREVKIRSENITNNAKNIIVQFVDPVEINCTRPNNNTRKSIHIGPGQAFYATGDIIGDIRQAHCNVSRSSWNKTLQQVAKQLGTYFKNKTIVFNTSSGGDPEITTHSFNCAGEFFYCDTSGLFNSSWNDTTWKESNSTGSNDTITLLCRIKQIINMWQRTGQCMYAPPIPGLISCKSNITGIILTRDGGNSHRTEETFRPGGGDMRDNWRSELYRYKVVQIEPLGVAPTRCRRRVVQGGSGGGGSGGGGSGGAVGIGAVFLGFLGAAGSTMGAASITLTVQARQLLSGIVQQQSNLLRAPEAQQHLLKLTVWGIKQLQARVLAVERYLRDQQLLGIWGCSGKLICCTNVPWNSSWSNKSYNDIWDNMTWLQWDKEIHNYTQLIYNLIEESQNQQEKNEQDLLALD

SEQ ID NO: 49 - protein, 866 aa, Artificial Sequence
  Synthetic construct
MPMGSLQPLATLYLLGMLVASVLATDNLWVTVYYGVPVWKDAETTLFCASDAKAYATEKHNVWATHACVPTDPNPQEIHLENVTEEFNMWKNNMVEQMHTDIISLWDQSLKPCVKLTPLCVTLSCSNAKVSYSNATVNNTIQDEIKNCSFNTTTVLRDKRQKVYSLFYRLDIVQIDNSSSDSSSSEYRLINCNTSACTQACPKVTFEPIPIHYCAPAGFAILKCKDEEFNGTGPCKNVSTVQCTHGIKPVVSTQLLLNGSLAKREVKIRSENITNNAKNIIVQFVDPVEINCTRPNNNTRKSIHIGPGQAFYATGDIIGDIRQAHCNVSRSSWNKTLQQVAKQLGTYFKNKTIVFNTSSGGDPEITTHSFNCAGEFFYCDTSGLFNSSWNDTTWKESNSTGSNDTITLLCRIKQIINMWQRTGQCMYAPPIPGLISCKSNITGIILTRDGGNSHRTEETFRPGGGDMRDNWRSELYRYKVVQIEPLGVAPTRCRRRVVQGGSGGGGSGGGGSGGAVGIGAVFLGFLGAAGSTMGAASITLTVQARQLLSGIVQQQSNLLRAPEAQQHLLKLTVWGIKQLQARVLAVERYLRDQQLLGIWGCSGKLICCTNVPWNSSWSNKSYNDIWDNMTWLQWDKEIHNYTQLIYNLIEESQNQQEKNEQDLLALDKWANLWNWFNITNWLWYIKIFIMVVGGLIGLRIVFAVLSIINRVRQGYSPLSFQTHLPNPRDLDRPERIEEEGGEQGRDRSIRLVSGFLALAWDDLRSLCLFSYHRLRDFILIAARTVELLGQSSLKGLRLGWESLKYLWNLLGYWVRELKISAVNLVDTIAIAVAGWTDRVIEIGQRIGRAIRHIPRRIRQGLERALL

SEQ ID NO: 50 - protein, 855 aa, Artificial Sequence
  Synthetic construct
MPMGSLQPLATLYLLGMLVASVLATDNLWVTVYYGVPVWKDAETTLFCASDAKAYATEKHNVWATHACVPTDPNPQEIHLENVTEEFNMWKNNMVEQMHTDIISLWDQSLKPCVKLTPLCVTLSCSNAKVSYSNATVNNTIQDEIKNCSFNTTTVLRDKRQKVYSLFYRLDIVQIDNSSSDSSSSEYRLINCNTSACTQACPKVTFEPIPIHYCAPAGFAILKCKDEEFNGTGPCKNVSTVQCTHGIKPVVSTQLLLNGSLAKREVKIRSENITNNAKNIIVQFVDPVEINCTRPNNNTRKSIHIGPGQAFYATGDIIGDIRQAHCNVSRSSWNKTLQQVAKQLGTYFKNKTIVFNTSSGGDPEITTHSFNCAGEFFYCDTSGLFNSSWNDTTWKESNSTGSNDTITLLCRIKQIINMWQRTGQCMYAPPIPGLISCKSNITGIILTRDGGNSHRTEETFRPGGGDMRDNWRSELYRYKVVQIEPLGVAPTRCRRRVVQGGSGGGGSGGGGSGGAVGIGAVFLGFLGAAGSTMGAASITLTVQARQLLSGIVQQQSNLLRAPEAQQHLLKLTVWGIKQLQARVLAVERYLRDQQLLGIWGCSGKLICCTNVPWNSSWSNKSYNDIWDNMTWLQWDKEIHNYTQLIYNLIEESQNQQEKNEQDLLALDGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 51 - protein, 866 aa, Artificial Sequence
  Synthetic construct
MRVMGTQRNCQHLLTWGTLILGIIIICSTAENLWVTVYYGVPVWRDADTTLFCASDAKAYETEKHNVWATHACVPTDPNPQEIDLKNVTEEFNMWKNNMVEQMHTDIISLWDQSLKPCVKLTPLCVTLNCSNANVTSENSTIMGDREEIKNCSFNMTTELRDKRQKVYSLFYRLDVVQINENQGNSSNNNYSEYRLINCNTSAITQACPKVSFEPIPIHYCAPAGFAILKCKDEEFNGTGPCKNVSTVQCTHGIKPVVSTQLLLNGSTAEKEIKIRSENITNNAKIIIVQLVKPVIINCTRPNNNTRRSVHIGPGQAFYATGDIIGNIRQAYCTVNRTDWNNTLQQVAKQLGKHFENKTIIFTKSSGGDLEITTHSFNCGGEFFYCNTSSLFNSTWSHNNSTLLGSNSTESNETITLPCRIKQIVNMWQRTGQAMYAPPIKGVIMCVSNITGLILTRDGGNDNSTNENETFRPGGGDMRDNWRSELYKYKVVQIEPLGVAPTRAKRRVVEREKRAVGIGAVFLGFLGAAGSTMGAASITLTVQARQLLSGIVRQQSNLLRAIEAQQHLLKLTVWGIKQLQARVLAVERYLRDQQLLGIWGCSGKLICTTSVPWNSSWSNKSLDEIWENMTWLQWEKEINNYTGLIYSLLEESQNQQEKNEQDLLALDKWANLWTWFGISNWLWYIRIFIIIVGGLIGLRIVFAVLSVVNRVRQGYSPLSFQIHPPNPGGLDRPGRIEEEGGEQGRDRSIRLVSGFLALAWDDLRSLCLFSYHRLRDFILIAARTVELLGHSSLKGLRLGWEGLKYLWNLLAYWGRELKISAISLVDNIAIVVAGWTDRVIEIGQGIGRAILHIPRRIRQGFERALL

SEQ ID NO: 52 - protein, 673 aa, Artificial Sequence
  Synthetic construct
MPMGSLQPLATLYLLGMLVASVLAAENLWVTVYYGVPVWRDADTTLFCASDAKAYETEKHNVWATHACVPTDPNPQEIDLKNVTEEFNMWKNNMVEQMHTDIISLWDQSLKPCVKLTPLCVTLNCSNANVTSENSTIMGDREEIKNCSFNMTTELRDKRQKVYSLFYRLDVVQINENQGNSSNNNYSEYRLINCNTSACTQACPKVSFEPIPIHYCAPAGFAILKCKDEEFNGTGPCKNVSTVQCTHGIKPVVSTQLLLNGSTAEKEIKIRSENITNNAKIIIVQLVKPVIINCTRPNNNTRRSVHIGPGQAFYATGDIIGNIRQAYCTVNRTDWNNTLQQVAKQLGKHFENKTIIFTKSSGGDLEITTHSFNCGGEFFYCNTSSLFNSTWSHNNSTLLGSNSTESNETITLPCRIKQIVNMWQRTGQCMYAPPIKGVIMCVSNITGLILTRDGGNDNSTNENETFRPGGGDMRDNWRSELYKYKVVQIEPLGVAPTRCKRRVVEGGSGGGGSGGGGSGGAVGIGAVFLGFLGAAGSTMGAASITLTVQARQLLSGIVRQQSNLLRAPEAQQHLLKLTVWGIKQLQARVLAVERYLRDQQLLGIWGCSGKLICCTSVPWNSSWSNKSLDEIWENMTWLQWEKEINNYTGLIYSLLEESQNQQEKNEQDLLALD

SEQ ID NO: 53 - protein, 872 aa, Artificial Sequence
  Synthetic construct
MPMGSLQPLATLYLLGMLVASVLAAENLWVTVYYGVPVWRDADTTLFCASDAKAYETEKHNVWATHACVPTDPNPQEIDLKNVTEEFNMWKNNMVEQMHTDIISLWDQSLKPCVKLTPLCVTLNCSNANVTSENSTIMGDREEIKNCSFNMTTELRDKRQKVYSLFYRLDVVQINENQGNSSNNNYSEYRLINCNTSACTQACPKVSFEPIPIHYCAPAGFAILKCKDEEFNGTGPCKNVSTVQCTHGIKPVVSTQLLLNGSTAEKEIKIRSENITNNAKIIIVQLVKPVIINCTRPNNNTRRSVHIGPGQAFYATGDIIGNIRQAYCTVNRTDWNNTLQQVAKQLGKHFENKTIIFTKSSGGDLEITTHSFNCGGEFFYCNTSSLFNSTWSHNNSTLLGSNSTESNETITLPCRIKQIVNMWQRTGQCMYAPPIKGVIMCVSNITGLILTRDGGNDNSTNENETFRPGGGDMRDNWRSELYKYKVVQIEPLGVAPTRCKRRVVEGGSGGGGSGGGGSGGAVGIGAVFLGFLGAAGSTMGAASITLTVQARQLLSGIVRQQSNLLRAPEAQQHLLKLTVWGIKQLQARVLAVERYLRDQQLLGIWGCSGKLICCTSVPWNSSWSNKSLDEIWENMTWLQWEKEINNYTGLIYSLLEESQNQQEKNEQDLLALDKWANLWTWFGISNWLWYIRIFIIIVGGLIGLRIVFAVLSVVNRVRQGYSPLSFQIHPPNPGGLDRPGRIEEEGGEQGRDRSIRLVSGFLALAWDDLRSLCLFSYHRLRDFILIAARTVELLGHSSLKGLRLGWEGLKYLWNLLAYWGRELKISAISLVDNIAIVVAGWTDRVIEIGQGIGRAILHIPRRIRQGFERALL

SEQ ID NO: 54 - protein, 851 aa, Artificial Sequence
  Synthetic construct
MPMGSLQPLATLYLLGMLVASVLAAENLWVTVYYGVPVWRDADTTLFCASDAKAYETEKHNVWATHACVPTDPNPQEIDLKNVTEEFNMWKNNMVEQMHTDIISLWDQSLKPCVKLTPLCVTLNCSNANVTSENSTIMGDREEIKNCSFNMTTELRDKRQKVYSLFYRLDVVQINENQGNSSNNNYSEYRLINCNTSACTQACPKVSFEPIPIHYCAPAGFAILKCKDEEFNGTGPCKNVSTVQCTHGIKPVVSTQLLLNGSTAEKEIKIRSENITNNAKIIIVQLVKPVIINCTRPNNNTRRSVHIGPGQAFYATGDIIGNIRQAYCTVNRTDWNNTLQQVAKQLGKHFENKTIIFTKSSGGDLEITTHSFNCGGEFFYCNTSSLFNSTWSHNNSTLLGSNSTESNETITLPCRIKQIVNMWQRTGQCMYAPPIKGVIMCVSNITGLILTRDGGNDNSTNENETFRPGGGDMRDNWRSELYKYKVVQIEPLGVAPTRCKRRVVEGGSGGGGSGGGGSGGAVGIGAVFLGFLGAAGSTMGAASITLTVQARQLLSGIVRQQSNLLRAPEAQQHLLKLTVWGIKQLQARVLAVERYLRDQQLLGIWGCSGKLICCTSVPWNSSWSNKSLDEIWENMTWLQWEKEINNYTGLIYSLLEESQNQQEKNEQDLLALDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 55 - protein, 854 aa, Artificial Sequence
  Synthetic construct
MRVMGIRKNYQHLWKWGTMLLWLLMICSAADQLWVTVYYGVPVWKEANTTLFCASDAKAYDTEAHNVWATHACVPTDPNPQEVVLENVTENFNMWKNHMVEQMHEDIISLWDQSLKPCVKLTPLCVTLNCTDLVNSNITRVDNTTEKEMKNCSFNVTSGIRDKVQKEYALLYKLDIVQIDNDNTSHRDNTSYRLISCNTSVITQACPKISFEPIPIHFCAPAGFAILKCNDKKFNGTGPCTNVSTVQCTHGIRPVVSTQLLLNGSLAEEEVVIRSENFTNNVKNIIVQLNESVQINCTRHNNNTRKSINIGPGRAFYATGKIIGDIRQAHCNISREKWQNTLKQIVKKLREQFKNKTIAFAPSSGGDPEIVMHSFNCNGEFFYCNTTKLFTSTWNSTWNSTWNNTEGSNSTVITLPCRIRQIINMWQEVGKAMYAPPIQGQIKCSSNITGLLLTRDGGVDTTKETFRPGGGNMKDNWRSELYKYKVVRIEPLGVAPTKAKRRVVQREKRAVGIGAMFLGFLGAAGSTMGAASITLTVQARLLLSGIVQQQSNLLRAIEAQQHLLQLTVWGIKQLQARVLAVERYLKDQQLLGIWGCSGKLICTTAVPWNASWSNKSQDTIWGNMTWMQWEREIDNYTDLIYNLLEESQNQQEKNEQELLALDKWASLWSWFSITHWLWYIKMFIMIVGGLVGLRIVFAVLSIVNRVRQGYSPLSFQTRFPAPRGPDRPEGIEEEGGERDRDRSGRSADGFLVLVWVDLRNLCLFSYHRLRDLLLIVTRTVELLGRRGWEALKYWWNLLQYWSQELKKSAVSLLDAIAIAVAEGTDRIIELLQRIFRAFLHIPTRIRQGLERALQ

SEQ ID NO: 56 - protein, 668 aa, Artificial Sequence
  Synthetic construct
MPMGSLQPLATLYLLGMLVASVLAADQLWVTVYYGVPVWKEANTTLFCASDAKAYDTEAHNVWATHACVPTDPNPQEVVLENVTENFNMWKNHMVEQMHEDIISLWDQSLKPCVKLTPLCVTLNCTDLVNSNITRVDNTTEKEMKNCSFNVTSGIRDKVQKEYALLYKLDIVQIDNDNTSHRDNTSYRLISCNTSVCTQACPKISFEPIPIHFCAPAGFAILKCNDKKFNGTGPCTNVSTVQCTHGIRPVVSTQLLLNGSLAEEEVVIRSENFTNNVKNIIVQLNESVQINCTRHNNNTRKSINIGPGRAFYATGKIIGDIRQAHCNISREKWQNTLKQIVKKLREQFKNKTIAFAPSSGGDPEIVMHSFNCNGEFFYCNTTKLFTSTWNSTWNSTWNNTEGSNSTVITLPCRIRQIINMWQEVGKCMYAPPIQGQIKCSSNITGLLLTRDGGVDTTKETFRPGGGNMKDNWRSELYKYKVVRIEPLGVAPTKCKRRVVQGGSGGGGSGGGGSGGAVGIGAMFLGFLGAAGSTMGAASITLTVQARLLLSGIVQQQSNLLRAPEAQQHLLQLTVWGIKQLQARVLAVERYLKDQQLLGIWGCSGKLICCTAVPWNASWSNKSQDTIWGNMTWMQWEREIDNYTDLIYNLLEESQNQQEKNEQELLALD

SEQ ID NO: 57 - protein, 860 aa, Artificial Sequence
  Synthetic construct
MPMGSLQPLATLYLLGMLVASVLAADQLWVTVYYGVPVWKEANTTLFCASDAKAYDTEAHNVWATHACVPTDPNPQEVVLENVTENFNMWKNHMVEQMHEDIISLWDQSLKPCVKLTPLCVTLNCTDLVNSNITRVDNTTEKEMKNCSFNVTSGIRDKVQKEYALLYKLDIVQIDNDNTSHRDNTSYRLISCNTSVCTQACPKISFEPIPIHFCAPAGFAILKCNDKKFNGTGPCTNVSTVQCTHGIRPVVSTQLLLNGSLAEEEVVIRSENFTNNVKNIIVQLNESVQINCTRHNNNTRKSINIGPGRAFYATGKIIGDIRQAHCNISREKWQNTLKQIVKKLREQFKNKTIAFAPSSGGDPEIVMHSFNCNGEFFYCNTTKLFTSTWNSTWNSTWNNTEGSNSTVITLPCRIRQIINMWQEVGKCMYAPPIQGQIKCSSNITGLLLTRDGGVDTTKETFRPGGGNMKDNWRSELYKYKVVRIEPLGVAPTKCKRRVVQGGSGGGGSGGGGSGGAVGIGAMFLGFLGAAGSTMGAASITLTVQARLLLSGIVQQQSNLLRAPEAQQHLLQLTVWGIKQLQARVLAVERYLKDQQLLGIWGCSGKLICCTAVPWNASWSNKSQDTIWGNMTWMQWEREIDNYTDLIYNLLEESQNQQEKNEQELLALDKWASLWSWFSITHWLWYIKMFIMIVGGLVGLRIVFAVLSIVNRVRQGYSPLSFQTRFPAPRGPDRPEGIEEEGGERDRDRSGRSADGFLVLVWVDLRNLCLFSYHRLRDLLLIVTRTVELLGRRGWEALKYWWNLLQYWSQELKKSAVSLLDAIAIAVAEGTDRIIELLQRIFRAFLHIPTRIRQGLERALQ

SEQ ID NO: 58 - protein, 846 aa, Artificial Sequence
  Synthetic construct
MPMGSLQPLATLYLLGMLVASVLAADQLWVTVYYGVPVWKEANTTLFCASDAKAYDTEAHNVWATHACVPTDPNPQEVVLENVTENFNMWKNHMVEQMHEDIISLWDQSLKPCVKLTPLCVTLNCTDLVNSNITRVDNTTEKEMKNCSFNVTSGIRDKVQKEYALLYKLDIVQIDNDNTSHRDNTSYRLISCNTSVCTQACPKISFEPIPIHFCAPAGFAILKCNDKKFNGTGPCTNVSTVQCTHGIRPVVSTQLLLNGSLAEEEVVIRSENFTNNVKNIIVQLNESVQINCTRHNNNTRKSINIGPGRAFYATGKIIGDIRQAHCNISREKWQNTLKQIVKKLREQFKNKTIAFAPSSGGDPEIVMHSFNCNGEFFYCNTTKLFTSTWNSTWNSTWNNTEGSNSTVITLPCRIRQIINMWQEVGKCMYAPPIQGQIKCSSNITGLLLTRDGGVDTTKETFRPGGGNMKDNWRSELYKYKVVRIEPLGVAPTKCKRRVVQGGSGGGGSGGGGSGGAVGIGAMFLGFLGAAGSTMGAASITLTVQARLLLSGIVQQQSNLLRAPEAQQHLLQLTVWGIKQLQARVLAVERYLKDQQLLGIWGCSGKLICCTAVPWNASWSNKSQDTIWGNMTWMQWEREIDNYTDLIYNLLEESQNQQEKNEQELLALDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 59 - protein, 846 aa, Artificial Sequence
  Synthetic construct
MRVRGIQTSWQNLWRWGTMILGMLMIYSAAENLWVTVYYGVPVWKDAETTLFCASDAKAYDTEVHNVWATHACVPTDPNPQEIHLENVTEDFNMWKNNMVEQMHTDIISLWDQSLKPCVKLTPLCVTLDCNATASNVTNEMRNCSFNITTELKDKKQQVYSLFYKLDVVQINEKNETDKYRLINCNTSAITQACPKVSFEPIPIHYCAPAGFAVLKCKDTEFNGTGPCKNVSTVQCTHGIRPVISTQLLLNGSLAEEGIQIRSENITNNAKTIIVQLDKAVKINCTRPNNNTRKGVRIGPGQAFYATGGIIGDIRQAHCNVSRAKWNDTLRGVAKKLREHFKNKTIIFEKSSGGDIEITTHSFNCGGEFFYCSTSGLFNSTWESNSTESNNTTSNDTITLTCRIKQIINMWQKVGQAMYAPPIQGVIRCESNITGLLLTRDGGNNSTNEIFRPGGGNMRDNWRSELYKYKVVKIEPLGVAPSRAKRRVVEREKRAVGIGAVFLGFLGAAGSTMGAASITLTAQARQLLSGIVQQQSNLLRAIEAQQHMLKLTVWGIKQLQARVLAVERYLKDQQLLGIWGCSGKLICTTNVPWNSSWSNKSMNEIWDNMTWLQWDKEISNYTQIIYNLIEESQNQQEKNEQDLLALDKWASLWNWFDISRWLWYIKIFIMIVGGLIGLRIVFAVLSVINRVRQGYSPLSFQIRTPNPKEPDRLGRIDGEGGEQDRDRSIRLVSGFLALAWDDLRSLCLFSYHRLRDFISIAARTVELLGHSSLKGLRLGWEGLKYLWNLLLYWGRELKTSAVNLVDTIAIAVAGWADRVMEVGQRIFRAILNIPRRIRQGLERGLL

SEQ ID NO: 60 - protein, 652 aa, Artificial Sequence
  Synthetic construct
MPMGSLQPLATLYLLGMLVASVLAENLWVTVYYGVPVWKDAETTLFCASDAKAYDTEVHNVWATHACVPTDPNPQEIHLENVTEDFNMWKNNMVEQMHTDIISLWDQSLKPCVKLTPLCVTLDCNATASNVTNEMRNCSFNITTELKDKKQQVYSLFYKLDVVQINEKNETDKYRLINCNTSACTQACPKVSFEPIPIHYCAPAGFAVLKCKDTEFNGTGPCKNVSTVQCTHGIRPVISTQLLLNGSLAEEGIQIRSENITNNAKTIIVQLDKAVKINCTRPNNNTRKGVRIGPGQAFYATGGIIGDIRQAHCNVSRAKWNDTLRGVAKKLREHFKNKTIIFEKSSGGDIEITTHSFNCGGEFFYCSTSGLFNSTWESNSTESNNTTSNDTITLTCRIKQIINMWQKVGQCMYAPPIQGVIRCESNITGLLLTRDGGNNSTNEIFRPGGGNMRDNWRSELYKYKVVKIEPLGVAPSRCKRRVVEGGSGGGGSGGGGSGGAVGIGAVFLGFLGAAGSTMGAASITLTAQARQLLSGIVQQQSNLLRAPEAQQHMLKLTVWGIKQLQARVLAVERYLKDQQLLGIWGCSGKLICCTNVPWNSSWSNKSMNEIWDNMTWLQWDKEISNYTQIIYNLIEESQNQQEKNEQDLLALD

SEQ ID NO: 61 - protein, 851 aa, Artificial Sequence
  Synthetic construct
MPMGSLQPLATLYLLGMLVASVLAENLWVTVYYGVPVWKDAETTLFCASDAKAYDTEVHNVWATHACVPTDPNPQEIHLENVTEDFNMWKNNMVEQMHTDIISLWDQSLKPCVKLTPLCVTLDCNATASNVTNEMRNCSFNITTELKDKKQQVYSLFYKLDVVQINEKNETDKYRLINCNTSACTQACPKVSFEPIPIHYCAPAGFAVLKCKDTEFNGTGPCKNVSTVQCTHGIRPVISTQLLLNGSLAEEGIQIRSENITNNAKTIIVQLDKAVKINCTRPNNNTRKGVRIGPGQAFYATGGIIGDIRQAHCNVSRAKWNDTLRGVAKKLREHFKNKTIIFEKSSGGDIEITTHSFNCGGEFFYCSTSGLFNSTWESNSTESNNTTSNDTITLTCRIKQIINMWQKVGQCMYAPPIQGVIRCESNITGLLLTRDGGNNSTNEIFRPGGGNMRDNWRSELYKYKVVKIEPLGVAPSRCKRRVVEGGSGGGGSGGGGSGGAVGIGAVFLGFLGAAGSTMGAASITLTAQARQLLSGIVQQQSNLLRAPEAQQHMLKLTVWGIKQLQARVLAVERYLKDQQLLGIWGCSGKLICCTNVPWNSSWSNKSMNEIWDNMTWLQWDKEISNYTQIIYNLIEESQNQQEKNEQDLLALDKWASLWNWFDISRWLWYIKIFIMIVGGLIGLRIVFAVLSVINRVRQGYSPLSFQIRTPNPKEPDRLGRIDGEGGEQDRDRSIRLVSGFLALAWDDLRSLCLFSYHRLRDFISIAARTVELLGHSSLKGLRLGWEGLKYLWNLLLYWGRELKTSAVNLVDTIAIAVAGWADRVMEVGQRIFRAILNIPRRIRQGLERGLL

SEQ ID NO: 62 - protein, 840 aa, Artificial Sequence
  Synthetic construct
MPMGSLQPLATLYLLGMLVASVLAENLWVTVYYGVPVWKDAETTLFCASDAKAYDTEVHNVWATHACVPTDPNPQEIHLENVTEDFNMWKNNMVEQMHTDIISLWDQSLKPCVKLTPLCVTLDCNATASNVTNEMRNCSFNITTELKDKKQQVYSLFYKLDVVQINEKNETDKYRLINCNTSACTQACPKVSFEPIPIHYCAPAGFAVLKCKDTEFNGTGPCKNVSTVQCTHGIRPVISTQLLLNGSLAEEGIQIRSENITNNAKTIIVQLDKAVKINCTRPNNNTRKGVRIGPGQAFYATGGIIGDIRQAHCNVSRAKWNDTLRGVAKKLREHFKNKTIIFEKSSGGDIEITTHSFNCGGEFFYCSTSGLFNSTWESNSTESNNTTSNDTITLTCRIKQIINMWQKVGQCMYAPPIQGVIRCESNITGLLLTRDGGNNSTNEIFRPGGGNMRDNWRSELYKYKVVKIEPLGVAPSRCKRRVVEGGSGGGGSGGGGSGGAVGIGAVFLGFLGAAGSTMGAASITLTAQARQLLSGIVQQQSNLLRAPEAQQHMLKLTVWGIKQLQARVLAVERYLKDQQLLGIWGCSGKLICCTNVPWNSSWSNKSMNEIWDNMTWLQWDKEISNYTQIIYNLIEESQNQQEKNEQDLLALDGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 63 - protein, 841 aa, Artificial Sequence
  Synthetic construct
MRVRGISRNWQQWWIWGVLGFWLLMSYSVLGNLWVTVYYGVPVWKEAKTTLFCASDAKAYEREVHNVWATHACVPTDPNPQEMVLENVTENFNMWKNDMVDQMHEDIISLWDQSLKPCVKLTPLCVTLNCTNASVNATYNGEMKNCSFNATTAIRDKKQQVRALFYSLDIVPLEGNNSSYRLISCNTSAITQACPKVSFDPIPIHYCTPAGYAILKCNDEKFNGTGPCHNVSTVQCTHGIKPVVSTQLLLNGSLAEKEIIIRSENLTNNAKTIIVHLNKAVEIVCVRPNNNTRKSIRIGPGQTFYANDIIGDIRQAHCNISESKWNDTLRQVGAKLAEHFNNNTIRFEPSSGGDLEITTHSFNCRGEFFYCNTSGLFNGTYNHTDTGGNSTNITLPCRIKQIINMWQEVGRAIYAPPVEGNIICISNITGLLLLRDGGHNSTNETFRPGGGDMRDNWRSELYKYKVVEIKPLGVAPTEAKRRVVEREKRAVGIGAMFLGFLGAAGSTMGAASITLTVQARQLLSGIVQQQSNLLRAIEAQQHMLQLTVWGIKQLQARVLSIERYLKDQQLLGLWGCSGKLICTTSVPWNHSWSNKSQKDIWENMTWMQWDREINNYTNTIYSLLEESQSQQEKNEKDLLALDNWNNLWNWFSITKWLWYIKIFIIIVGGLIGLRIIFAVLSIVNRVRQGYSPLSLQTLIPSPRGPDRLGRIEEEGGEQDKDRSIRLVSGFLSLAWDDLRSLCLFSYHRLRDFLLVTARAVELLGRSSLKGLQKGWEALKYLGNLVQYWGLELKKSVISLIDIIAIAVAEGTDRIIEVIQRICRAIRNIPTRIRQGFETALL

SEQ ID NO: 64 - protein, 646 aa, Artificial Sequence
  Synthetic construct
MPMGSLQPLATLYLLGMLVASVLANLWVTVYYGVPVWKEAKTTLFCASDAKAYEREVHNVWATHACVPTDPNPQEMVLENVTENFNMWKNDMVDQMHEDIISLWDQSLKPCVKLTPLCVTLNCTNASVNATYNGEMKNCSFNATTAIRDKKQQVRALFYSLDIVPLEGNNSSYRLISCNTSACTQACPKVSFDPIPIHYCTPAGYAILKCNDEKFNGTGPCHNVSTVQCTHGIKPVVSTQLLLNGSLAEKEIIIRSENLTNNAKTIIVHLNKAVEIVCVRPNNNTRKSIRIGPGQTFYANDIIGDIRQAHCNISESKWNDTLRQVGAKLAEHFNNNTIRFEPSSGGDLEITTHSFNCRGEFFYCNTSGLFNGTYNHTDTGGNSTNITLPCRIKQIINMWQEVGRCIYAPPVEGNIICISNITGLLLLRDGGHNSTNETFRPGGGDMRDNWRSELYKYKVVEIKPLGVAPTECKRRVVEGGSGGGGSGGGGSGGAVGIGAMFLGFLGAAGSTMGAASITLTVQARQLLSGIVQQQSNLLRAPEAQQHMLQLTVWGIKQLQARVLSIERYLKDQQLLGLWGCSGKLICCTSVPWNHSWSNKSQKDIWENMTWMQWDREINNYTNTIYSLLEESQSQQEKNEKDLLALD

SEQ ID NO: 65 - protein, 845 aa, Artificial Sequence
  Synthetic construct
MPMGSLQPLATLYLLGMLVASVLANLWVTVYYGVPVWKEAKTTLFCASDAKAYEREVHNVWATHACVPTDPNPQEMVLENVTENFNMWKNDMVDQMHEDIISLWDQSLKPCVKLTPLCVTLNCTNASVNATYNGEMKNCSFNATTAIRDKKQQVRALFYSLDIVPLEGNNSSYRLISCNTSACTQACPKVSFDPIPIHYCTPAGYAILKCNDEKFNGTGPCHNVSTVQCTHGIKPVVSTQLLLNGSLAEKEIIIRSENLTNNAKTIIVHLNKAVEIVCVRPNNNTRKSIRIGPGQTFYANDIIGDIRQAHCNISESKWNDTLRQVGAKLAEHFNNNTIRFEPSSGGDLEITTHSFNCRGEFFYCNTSGLFNGTYNHTDTGGNSTNITLPCRIKQIINMWQEVGRCIYAPPVEGNIICISNITGLLLLRDGGHNSTNETFRPGGGDMRDNWRSELYKYKVVEIKPLGVAPTECKRRVVEGGSGGGGSGGGGSGGAVGIGAMFLGFLGAAGSTMGAASITLTVQARQLLSGIVQQQSNLLRAPEAQQHMLQLTVWGIKQLQARVLSIERYLKDQQLLGLWGCSGKLICCTSVPWNHSWSNKSQKDIWENMTWMQWDREINNYTNTIYSLLEESQSQQEKNEKDLLALDNWNNLWNWFSITKWLWYIKIFIIIVGGLIGLRIIFAVLSIVNRVRQGYSPLSLQTLIPSPRGPDRLGRIEEEGGEQDKDRSIRLVSGFLSLAWDDLRSLCLFSYHRLRDFLLVTARAVELLGRSSLKGLQKGWEALKYLGNLVQYWGLELKKSVISLIDIIAIAVAEGTDRIIEVIQRICRAIRNIPTRIRQGFETALL

SEQ ID NO: 66 - protein, 834 aa, Artificial Sequence
  Synthetic construct
MPMGSLQPLATLYLLGMLVASVLANLWVTVYYGVPVWKEAKTTLFCASDAKAYEREVHNVWATHACVPTDPNPQEMVLENVTENFNMWKNDMVDQMHEDIISLWDQSLKPCVKLTPLCVTLNCTNASVNATYNGEMKNCSFNATTAIRDKKQQVRALFYSLDIVPLEGNNSSYRLISCNTSACTQACPKVSFDPIPIHYCTPAGYAILKCNDEKFNGTGPCHNVSTVQCTHGIKPVVSTQLLLNGSLAEKEIIIRSENLTNNAKTIIVHLNKAVEIVCVRPNNNTRKSIRIGPGQTFYANDIIGDIRQAHCNISESKWNDTLRQVGAKLAEHFNNNTIRFEPSSGGDLEITTHSFNCRGEFFYCNTSGLFNGTYNHTDTGGNSTNITLPCRIKQIINMWQEVGRCIYAPPVEGNIICISNITGLLLLRDGGHNSTNETFRPGGGDMRDNWRSELYKYKVVEIKPLGVAPTECKRRVVEGGSGGGGSGGGGSGGAVGIGAMFLGFLGAAGSTMGAASITLTVQARQLLSGIVQQQSNLLRAPEAQQHMLQLTVWGIKQLQARVLSIERYLKDQQLLGLWGCSGKLICCTSVPWNHSWSNKSQKDIWENMTWMQWDREINNYTNTIYSLLEESQSQQEKNEKDLLALDGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 67 - protein, 1336 aa, Artificial Sequence
  Synthetic construct
MPMGSLQPLATLYLLGMLVASVLAGEDLWVTVYYGVPVWKEANPTLFCASDAKAYKTEMHNVWATHACVPTDPNPQEMVLENVTEDFNMWKNGMVEQMHQDIISLWDQSLKPCVKLTPLCVTLNCTEVTRSSNGTINNNSTEMKNCSFNVTTDLRDKKKKEHALFYRLDIVPLDETNGTSSEYRLINCNTSTCTQACPKVSFDPIPIHYCAPAGYAILKCKDKKFNGTGPCKNVSTVQCTHGIKPVVSTQLLLNGSIAEGEIIIRSENLTNNAKIIIVQLNVTVEINCTRPNNNTRRSIRIGPGQTFYATGEIIGDIRQAHCNISREKWNRTLQKVEKKLEELFPNKTIHFTSSSGGDLEITTHSFNCMGEFFYCNTSALFNNNNDSTNSNITLPCRIRQFINMWQEVGRCMYAPPIQGVITCKSNVTGLLLTRDGGIINDTEIFRPGGGDMRDNWRSELYKYKVVEIKPLGIAPTTCKRRVVEGGSGGGGSGGGGSGGAVGIGAVFLGFLGAAGSTMGAASITLTAQARQLLSGIVQQQSNLLRAPEAQQHMLQLTVWGIKQLQTRVLAIERYLKDQQLLGIWGCSGKLICCTAVPWNGSWSNKSQDEIWHNMTWMQWDKEINNYTNIIYGLLEVSQNQQEKNEQDLLALDGSGATNFSLLKQAGDVEENPGPGSGMKAKLLVLLCTFTATYAGNLWVTVYYGVPVWKEAKTTLFCASDAKAHKEEVHNIWATHACVPTDPNPQEIVLKNVTENFNMWKNDMVDQMHEDIISLWDQSLKPCVKLTPLCVTLNCSDVKIKGTNATYNNATYNNNNTISDMKNCSFNTTTEITDKKKKEYALFYKLDVVALDGKETNSTNSSEYRLINCNTSACTQACPKVSFDPIPIHYCAPAGYAILKCNNKTFNGTGPCNNVSTVQCTHGIKPVVSTQLLLNGSLAEEEVVIRFENLTNNAKIIIVHLNESVEINCTRPSNNTRKSVRIGPGQTFFATGDIIGDIRQAHCNISRKKWNTTLQRVKEKLKEKFPNKTIQFAPSSGGDLEITTHSFNCRGEFFYCYTSDLFNSTYMSNNTGGANITLQCRIKQIIRMWQGVGQCMYAPPIAGNITCKSNITGLLLTRDGGKEKNDTETFRPGGGDMRDNWRSELYKYKVVEIKPLGIAPDKCKRRVVEGGSGGGGSGGGGSGGAVGIGAVFLGFLGAAGSTMGAASMTLTVQARQLLSGIVQQQSNLLRAPEAQQHMLQLTVWGIKQLQTRVLAIERYLKDQQLLGIWGCSGKLICCTAVPWNASWSNKSYEEIWGNMTWMQWDREINNYTNTIYSLLEESQNQQEKNEKDLLALD

SEQ ID NO: 68 - protein, 1564 aa, Artificial Sequence
  Synthetic construct
MPMGSLQPLATLYLLGMLVASVLAAENLWVTVYYGVPVWRDADTTLFCASDAKAYETEKHNVWATHACVPTDPNPQEIDLKNVTEEFNMWKNNMVEQMHTDIISLWDQSLKPCVKLTPLCVTLNCSNANVTSENSTIMGDREEIKNCSFNMTTELRDKRQKVYSLFYRLDVVQINENQGNSSNNNYSEYRLINCNTSACTQACPKVSFEPIPIHYCAPAGFAILKCKDEEFNGTGPCKNVSTVQCTHGIKPVVSTQLLLNGSTAEKEIKIRSENITNNAKIIIVQLVKPVIINCTRPNNNTRRSVHIGPGQAFYATGDIIGNIRQAYCTVNRTDWNNTLQQVAKQLGKHFENKTIIFTKSSGGDLEITTHSFNCGGEFFYCNTSSLFNSTWSHNNSTLLGSNSTESNETITLPCRIKQIVNMWQRTGQCMYAPPIKGVIMCVSNITGLILTRDGGNDNSTNENETFRPGGGDMRDNWRSELYKYKVVQIEPLGVAPTRCKRRVVEGGSGGGGSGGGGSGGAVGIGAVFLGFLGAAGSTMGAASITLTVQARQLLSGIVRQQSNLLRAPEAQQHLLKLTVWGIKQLQARVLAVERYLRDQQLLGIWGCSGKLICCTSVPWNSSWSNKSLDEIWENMTWLQWEKEINNYTGLIYSLLEESQNQQEKNEQDLLALDGSGATNFSLLKQAGDVEENPGPGSGMPMGSLQPLATLYLLGMLVASVLATDNLWVTVYYGVPVWKDAETTLFCASDAKAYATEKHNVWATHACVPTDPNPQEIHLENVTEEFNMWKNNMVEQMHTDIISLWDQSLKPCVKLTPLCVTLSCSNAKVSYSNATVNNTIQDEIKNCSFNTTTVLRDKRQKVYSLFYRLDIVQIDNSSSDSSSSEYRLINCNTSACTQACPKVTFEPIPIHYCAPAGFAILKCKDEEFNGTGPCKNVSTVQCTHGIKPVVSTQLLLNGSLAKREVKIRSENITNNAKNIIVQFVDPVEINCTRPNNNTRKSIHIGPGQAFYATGDIIGDIRQAHCNVSRSSWNKTLQQVAKQLGTYFKNKTIVFNTSSGGDPEITTHSFNCAGEFFYCDTSGLFNSSWNDTTWKESNSTGSNDTITLLCRIKQIINMWQRTGQCMYAPPIPGLISCKSNITGIILTRDGGNSHRTEETFRPGGGDMRDNWRSELYRYKVVQIEPLGVAPTRCRRRVVQGGSGGGGSGGGGSGGAVGIGAVFLGFLGAAGSTMGAASITLTVQARQLLSGIVQQQSNLLRAPEAQQHLLKLTVWGIKQLQARVLAVERYLRDQQLLGIWGCSGKLICCTNVPWNSSWSNKSYNDIWDNMTWLQWDKEIHNYTQLIYNLIEESQNQQEKNEQDLLALDKWANLWNWFNITNWLWYIKIFIMVVGGLIGLRIVFAVLSIINRVRQGYSPLSFQTHLPNPRDLDRPERIEEEGGEQGRDRSIRLVSGFLALAWDDLRSLCLFSYHRLRDFILIAARTVELLGQSSLKGLRLGWESLKYLWNLLGYWVRELKISAVNLVDTIAIAVAGWTDRVIEIGQRIGRAIRHIPRRIRQGLERALL

SEQ ID NO: 69 - protein, 1390 aa, Artificial Sequence
  Synthetic construct
MPMGSLQPLATLYLLGMLVASVLATEKLWVTVYYGVPVWKEATTTLFCASDAKAYETEVHNVWATHACVPTDPNPQEVLLENVTENFNMWKNNMVEQMQEDIISLWDQSLKPCVKLTPLCVTLECHDVNVNGTANNGTTNVTESGVNSSDVTSNNVTNSNWGTMEKGEIKNCSFNITTNIRDKMQKETAQFYKLDIVPIEDQNKTNNTLYRLINCNTSVCTQACPKVSFEPIPIHYCTPAGFAILKCNDRNFNGTGPCKNVSTVQCTHGIKPVVSTQLLLNGSLAEAEVVIRSENFTNNAKTIIIQLNETVEINCTRPNNNTSKRISIGPGRAFRATKIIGNIRQAHCNISRATWNSTLKKIVAKLREQFGNKTIVFQPSSGGDPEIVMHSFNCGGEFFYCNTTQLFNSTWNSTEESNSTEEGTITLPCRIKQIINMWQEVGKCMYAPPIEGQIRCSSNITGLLLTRDGGNNNKTNGTEIFRPGGGDMRDNWRSELYKYKVVKIEPLGVAPTKCKRRVVQGGSGGGGSGGGGSGGAVGIVGAMFLGFLGAAGSTMGAASMTLTVQARLLLSGIVQQQNNLLRAPEAQQHLLQLTVWGIKQLQARVLAVERYLKDQQLLGIWGCSGKLICCTTVPWNTSWSNKSLSEIWDNMTWMQWEREIDNYTSLIYTLIEESQNQQEKNEQELLELDGSGATNFSLLKQAGDVEENPGPGSGMPMGSLQPLATLYLLGMLVASVLAADKLWVTVYYGVPVWKETTTTLFCASDARAYDTEVHNVWATHACVPTDPNPQEVVLGNVTENFNMWKNNMVEQMHEDIISLWDQSLKPCVRLTPLCVTLNCIDYYGNTTNSNNSSETMMEKGEIKNCSFNITTRLKDKMQKEYALFYKYDIVPIDNRVGNDTSNATSYRLTSCNTSVCTQACPKVSFEPIPIHYCAPAGFAILKCNDKKFNGTGPCKNVSTVQCTHGIKPVVSTQLLLNGSLAEEEVMIRSENFTDNAKTIIVQLNETVEINCTRPNNNTRKSIHMGPGKVFYTTGEIIGDIRQAHCNINRAKWNNTLIKIVEKLRVKFNKTISFKQSSGGDPEIEMHSFNCGGEFFYCNTTQLFNSTWFNNATLNVNSNVTEGSENITLPCRIRQIVNMWQEVGKCMYAPPIQGQIRCSSNITGLLLTRDGGGSNSSNTSEEVFRPGGGNMRDNWRSELYKYKVVKIEPLGIAPTKCKRRVVQGGSGGGGSGGGGSGGTVGIGALFLGFLGAAGSTMGAASMTLTVQARQLLSGIVQQQNNLLRAPQAQQHLLQLTVWGIKQLQARVLAVERYLKDQQLLGIWGCSGKLICCTAVPWNASWSNKSLNEIWDNMTWMEWEKEIDNYTSLIYTLIEESQNQQEKNEQELLELD

SEQ ID NO: 70 - protein, 1702 aa, Artificial Sequence
  Synthetic construct
MPMGSLQPLATLYLLGMLVASVLAGEDLWVTVYYGVPVWKEANPTLFCASDAKAYKTEMHNVWATHACVPTDPNPQEMVLENVTEDFNMWKNGMVEQMHQDIISLWDQSLKPCVKLTPLCVTLNCTEVTRSSNGTINNNSTEMKNCSFNVTTDLRDKKKKEHALFYRLDIVPLDETNGTSSEYRLINCNTSTCTQACPKVSFDPIPIHYCAPAGYAILKCKDKKFNGTGPCKNVSTVQCTHGIKPVVSTQLLLNGSIAEGEIIIRSENLTNNAKIIIVQLNVTVEINCTRPNNNTRRSIRIGPGQTFYATGEIIGDIRQAHCNISREKWNRTLQKVEKKLEELFPNKTIHFTSSSGGDLEITTHSFNCMGEFFYCNTSALFNNNNDSTNSNITLPCRIRQFINMWQEVGRCMYAPPIQGVITCKSNVTGLLLTRDGGIINDTEIFRPGGGDMRDNWRSELYKYKVVEIKPLGIAPTTCKRRVVEGGSGGGGSGGGGSGGAVGIGAVFLGFLGAAGSTMGAASITLTAQARQLLSGIVQQQSNLLRAPEAQQHMLQLTVWGIKQLQTRVLAIERYLKDQQLLGIWGCSGKLICCTAVPWNGSWSNKSQDEIWHNMTWMQWDKEINNYTNIIYGLLEVSQNQQEKNEQDLLALDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVGSGATNFSLLKQAGDVEENPGPGSGMKAKLLVLLCTFTATYAGNLWVTVYYGVPVWKEAKTTLFCASDAKAHKEEVHNIWATHACVPTDPNPQEIVLKNVTENFNMWKNDMVDQMHEDIISLWDQSLKPCVKLTPLCVTLNCSDVKIKGTNATYNNATYNNNNTISDMKNCSFNTTTEITDKKKKEYALFYKLDVVALDGKETNSTNSSEYRLINCNTSACTQACPKVSFDPIPIHYCAPAGYAILKCNNKTFNGTGPCNNVSTVQCTHGIKPVVSTQLLLNGSLAEEEVVIRFENLTNNAKIIIVHLNESVEINCTRPSNNTRKSVRIGPGQTFFATGDIIGDIRQAHCNISRKKWNTTLQRVKEKLKEKFPNKTIQFAPSSGGDLEITTHSFNCRGEFFYCYTSDLFNSTYMSNNTGGANITLQCRIKQIIRMWQGVGQCMYAPPIAGNITCKSNITGLLLTRDGGKEKNDTETFRPGGGDMRDNWRSELYKYKVVEIKPLGIAPDKCKRRVVEGGSGGGGSGGGGSGGAVGIGAVFLGFLGAAGSTMGAASMTLTVQARQLLSGIVQQQSNLLRAPEAQQHMLQLTVWGIKQLQTRVLAIERYLKDQQLLGIWGCSGKLICCTAVPWNASWSNKSYEEIWGNMTWMQWDREINNYTNTIYSLLEESQNQQEKNEKDLLALDGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 71 - protein, 1731 aa, Artificial Sequence
  Synthetic construct
MPMGSLQPLATLYLLGMLVASVLAAENLWVTVYYGVPVWRDADTTLFCASDAKAYETEKHNVWATHACVPTDPNPQEIDLKNVTEEFNMWKNNMVEQMHTDIISLWDQSLKPCVKLTPLCVTLNCSNANVTSENSTIMGDREEIKNCSFNMTTELRDKRQKVYSLFYRLDVVQINENQGNSSNNNYSEYRLINCNTSACTQACPKVSFEPIPIHYCAPAGFAILKCKDEEFNGTGPCKNVSTVQCTHGIKPVVSTQLLLNGSTAEKEIKIRSENITNNAKIIIVQLVKPVIINCTRPNNNTRRSVHIGPGQAFYATGDIIGNIRQAYCTVNRTDWNNTLQQVAKQLGKHFENKTIIFTKSSGGDLEITTHSFNCGGEFFYCNTSSLFNSTWSHNNSTLLGSNSTESNETITLPCRIKQIVNMWQRTGQCMYAPPIKGVIMCVSNITGLILTRDGGNDNSTNENETFRPGGGDMRDNWRSELYKYKVVQIEPLGVAPTRCKRRVVEGGSGGGGSGGGGSGGAVGIGAVFLGFLGAAGSTMGAASITLTVQARQLLSGIVRQQSNLLRAPEAQQHLLKLTVWGIKQLQARVLAVERYLRDQQLLGIWGCSGKLICCTSVPWNSSWSNKSLDEIWENMTWLQWEKEINNYTGLIYSLLEESQNQQEKNEQDLLALDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVGSGATNFSLLKQAGDVEENPGPGSGMPMGSLQPLATLYLLGMLVASVLATDNLWVTVYYGVPVWKDAETTLFCASDAKAYATEKHNVWATHACVPTDPNPQEIHLENVTEEFNMWKNNMVEQMHTDIISLWDQSLKPCVKLTPLCVTLSCSNAKVSYSNATVNNTIQDEIKNCSFNTTTVLRDKRQKVYSLFYRLDIVQIDNSSSDSSSSEYRLINCNTSACTQACPKVTFEPIPIHYCAPAGFAILKCKDEEFNGTGPCKNVSTVQCTHGIKPVVSTQLLLNGSLAKREVKIRSENITNNAKNIIVQFVDPVEINCTRPNNNTRKSIHIGPGQAFYATGDIIGDIRQAHCNVSRSSWNKTLQQVAKQLGTYFKNKTIVFNTSSGGDPEITTHSFNCAGEFFYCDTSGLFNSSWNDTTWKESNSTGSNDTITLLCRIKQIINMWQRTGQCMYAPPIPGLISCKSNITGIILTRDGGNSHRTEETFRPGGGDMRDNWRSELYRYKVVQIEPLGVAPTRCRRRVVQGGSGGGGSGGGGSGGAVGIGAVFLGFLGAAGSTMGAASITLTVQARQLLSGIVQQQSNLLRAPEAQQHLLKLTVWGIKQLQARVLAVERYLRDQQLLGIWGCSGKLICCTNVPWNSSWSNKSYNDIWDNMTWLQWDKEIHNYTQLIYNLIEESQNQQEKNEQDLLALDGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

(19) United States
(12) Patent Application Publication
(10) Pub. No.: US 2023/0145420 A1
(43) Pub. Date: May 11, 2023

(54) RECOMBINANT VACCINES AND METHODS OF USE THEREOF

(71) Applicant: Vanderbilt University, Nashville, TN (US)

(72) Inventors: Matthew J. Vukovich, Nashville, TN (US); Nagarajan Raju, Nashville, TN (US); Amyn Murji, Nashville, TN (US); Ivelin Stefanov Georgiev, Brentwood, TN (US)

(21) Appl. No.: 17/917,651

(22) PCT Filed: Apr. 9, 2021
(86) PCT No.: PCT/US2021/026572

§ 371 Date: Oct. 7, 2022

Related U.S. Application Data
(60) Provisional application No. 63/007,985, filed on Apr. 10, 2020.
(60) Provisional application No. 63/007,989, filed on Apr. 10, 2020.

(51) Int. Cl. A61K 39/21; C07K 14/005; C07K 14/47; A61P 31/18

(52) U.S. Cl. CPC A61K 39/21; C07K 14/005; C07K 14/47; A61P 31/18; A61K 39/00

(57) ABSTRACT

The present disclosure relates to recombinant nucleic acids and uses thereof for developing vaccines.

### CROSS-REFERENCE TO RELATED APPLICATIONS

This application claims the benefit of U.S. Provisional Application No. 63/007,985 filed Apr. 10, 2020, and U.S. Provisional Application No. 63/007,989, filed Apr. 10, 2020, which are expressly incorporated herein by reference in their entireties.

### FIELD

The present disclosure relates to recombinant nucleic acids and use thereof for making vaccines.

### BACKGROUND

HIV-1 continues to impose a large global health burden. Candidate vaccines using HIV-derived antigens have not proven effective to date, and efforts toward protection against new infections remain a high priority in HIV-1 research. In recent years, strategies that target the elicitation of broadly neutralizing antibodies that are capable of neutralizing a large fraction of circulating HIV-1 variants have emerged as a potential avenue to a prophylactic HIV-1 vaccine. The sole target of these neutralizing antibodies is the envelope protein (Env) of HIV-1. However, due to the extensive global diversity of HIV-1, Env-based vaccine candidates so far have only led to the elicitation of antibodies with limited neutralization breadth. Therefore, what is needed are platforms for developing new vaccines that elicit an antibody response with broad neutralization breadth.

### SUMMARY

Disclosed herein are recombinant nucleic acids and uses thereof for producing vaccines (e.g., DNA vaccines, RNA vaccines, protein vaccines, and nanoparticle vaccines). The recombinant nucleic acids enable the production of vaccines with broad neutralization breath against multiple antigens derived from one or more strains/clades/mutant of a pathogen. Also disclosed herein are methods of treating and/or preventing infection (e.g., viral infection, bacterial infection, parasitic infection, or fungal infection) using the vaccines disclosed herein.

In some aspects, disclosed herein is a recombinant nucleic acid comprising two or more polynucleotide sequences encoding two or more antigens, wherein the 3′ end of each of the two or more polynucleotide sequences encoding the two or more antigens is operably linked to a 2A polynucleotide sequence.

In some embodiments, the 2A polynucleotide sequence encodes a 2A polypeptide that is self-cleavage. In some embodiments, the 5′ end of each of the two or more polynucleotides encoding the two or more antigens is operably linked to a polynucleotide sequence encoding a signal peptide. In some embodiments, the two or more antigens are antigens of pathogens. In some embodiments, the antigens are viral antigens. In some embodiments, the viral antigens are HIV antigens, influenza antigens, or SARS-CoV-2 antigens. In some embodiments, the HIV antigens are HIV Env proteins or HIV fusion peptides. In some embodiments, the polynucleotide sequence encoding the HIV antigen comprises a sequence at least about 90% identical to SEQ ID NO: 5 or 7.

In some embodiments, the polynucleotide sequence encoding the signal peptide comprises a sequence at least about 90% identical to SEQ ID NO: 15.

In some embodiments, the 2A polynucleotide sequence comprises a sequence at least about 90% identical to SEQ ID NO: 11 or 12.

In some embodiments, the polynucleotide sequence encoding the signal peptide, the polynucleotide sequence encoding the antigen, and the 2A polynucleotide sequence are operably linked. In some embodiments, the recombinant nucleic acid further comprises a polynucleotide sequence encoding a ferritin protein. In some embodiments, the polynucleotide sequence encoding the ferritin protein is operably linked to the 3′ end of each of the two or more of the polynucleotide sequences encoding the two or more antigens and to the 5′ end of the 2A polynucleotide sequence.

In some embodiments, the recombinant nucleic acid comprises a sequence at least about 90% identical to SEQ ID NO: 1 or 3.

In some aspects, disclosed herein is a DNA vaccine comprising the recombinant nucleic acid of any preceding aspect.

In some aspects, disclosed herein is an RNA vaccine comprising a sequence that is transcribed from the recombinant nucleic acid of any preceding aspect.

In some aspects, disclosed herein is a method of preventing and/or treating an infection in a subject, comprising administering to the subject an effective amount of the vaccine disclosed herein.

Also disclosed herein is a recombinant nucleic acid comprising two or more polynucleotide sequences encoding two or more antigens, wherein the 3′ end of each of the two or more polynucleotide sequences encoding the two or more antigens is operably linked to a polynucleotide sequence encoding a ferritin protein and a 2A polynucleotide sequence.

In some embodiments, the 2A polynucleotide sequence encodes a 2A polypeptide that is self-cleavage. In some embodiments, the polynucleotide sequence encoding the ferritin protein is operably linked to the 3′ end of each of the two or more of the polynucleotide sequences encoding the two or more antigens and to the 5′ end of the 2A polynucleotide sequence.

In some embodiments, the antigens are viral antigens. In some embodiments, the viral antigens are HIV antigens, influenza antigens, or SARS-CoV-2 antigens. In some embodiments, the HIV antigens are HIV Env proteins or HIV fusion peptides. In some embodiments, the HIV antigens are derived from two or more clades of HIV (e.g., BG505 and/or CZA97).

In some aspects, disclosed herein is a nanoparticle vaccine encoded by the recombinant nucleic acid of any preceding aspect.

In some aspects, disclosed herein is a method of preventing and/or treating HIV infection in a subject, comprising administering to the subject an effective amount of the nanoparticle vaccine disclosed herein.

### BRIEF DESCRIPTION OF THE DRAWINGS

The accompanying figures, which are incorporated in and constitute a part of this specification, illustrate several aspects described below.

FIG. 1 shows structures of HIV-1 Env by common epitopes.

FIGS. 2A-2D show the vaccine platforms. FIG. 2A shows analysis of nanoparticles from phage MS2 capsid. Negative-stain EM shows the formation of particles of the expected size. FIG. 2B shows structural model of an antigen (colored spikes) on a ferritin particle (green). The antigen can be fused to either the N- or C-terminus of the particle protein. FIG. 2C shows successful expression and purification of HIV-1 Env trimers to be used as part of cocktail immunogens in animal studies. FIG. 2D shows expression of ferritin nanoparticle immunogens mounted with HIV-1 Env proteins.

FIGS. 3A-3B show 2A peptide generated antigens. FIG. 3A shows schematic of multiantigen DNA using 2A peptides as separators between the different antigen genes. 2A peptides are typically short segments (˜20 amino acids in length) that promote ribosome skipping and therefore act as “self-cleaving” agents to result in multiple protein products from a single gene construct. This technology can be implemented in delivering a DNA vaccine.

FIG. 3B shows ELISAs validating expression of multiple Envs from a single transcript. Antibodies specific to each Env trimer variant were used to identify expression of each Env.

FIGS. 4A-4C show animal studies. FIG. 4A shows immunization groups. Trimer cocktails, nanoparticle cocktails and co-expressed nanoparticles were used to intramuscularly immunize BALB/c mice. Mice were exsanguinated at day 70 for serological analyses. FIG. 4B shows immunizations with nanoparticles elicit comparable antibody titers when compared to titers elicited in response to immunizations with trimer cocktails. FIG. 4C shows antigen specific B-cell sorting shows B-cells that are cross-reactive to the two trimers in the vaccine.

FIGS. 5A-5B show study indicating heterologous breadth. FIG. 5A shows mouse sera showing neutralization against a heterologous Tier 2 virus, Ce1176. FIG. 5B shows that nanoparticles were used to immunize guinea pigs.

FIGS. 6A-6C show expression and characterization of a fusion-peptide nanoparticle vaccine. FIG. 6A shows the fusion peptide of HIV-1 is relatively conserved. Selection of fusion peptides should incorporate maximum diversity in order to cover the majority of circulating strains. FIG. 6B shows successful expression of fusion-peptide-ferritin is evident from negative-stain EM. FIG. 6C shows that fusion peptide nanoparticles are recognized by monoclonal antibody VRC34.01 as evidenced by negative-stain EM and ELISA. This antibody binds to the fusion peptide of HIV-1.

FIGS. 7A-7D show successful expression and characterization of nanoparticle immunogens from 2A constructs. FIG. 7A shows schematic of multi-antigen DNA using 2A peptides as separators between BG505-ferritin and CZA97-ferritin genes. FIG. 7B shows that BG505 was mutated to abrogate binding of monoclonal antibody 10-1074 in single-antigen and multi-antigen 2A constructs. PG16 does not bind CZA97. Expression of BG505-Ferrtin.2A.CZA97-Ferritin validates expression of both antigens from 2A construct. FIG. 7C shows that trimer-nanoparticles are first purified on a Galanthus nivalis lectin column, followed by size-exclusion on a HiPrep 16/60 Sephacryl S-500HR column. The protein eluted within the expected range (60-80 mls). FIG. 7D shows negative stain EM images that confirm expression from 2A constructs yield fully formed trimer-nanoparticle immunogens.

FIGS. 8A-8D show successful expression and characterization of nanoparticle immunogens from 2A constructs. FIG. 8A shows schematic of multi-antigen DNA using 2A peptides as separators between one fusion-peptide-ferritin variant gene and a second, different fusion-peptide-variant gene. FIG. 8B shows that expression of fusion-peptide-nanoparticles requires purification over a VRC34.01 affinity column. Pure protein elutes between fraction 3 and 5. Fractions are collected and run on a Superdex 200Increase 10/300 GL column. The protein eluted within the expected range (12-14 mls), which is the expected volume. FIG. 8C shows immunogens from the 2A construct recognize VRC34.01. FP2 is not recognized by VRC34 and serves as a negative control. FIG. 8D shows negative stain EM images that confirm expression from 2A constructs. Purified protein exhibit as fully formed nanoparticles (left) and recognize VRC34.01 (right).

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below. As used herein, the article “a,” “an,” and “the” means “at least one,” unless the context in which the article is used clearly indicates otherwise.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The terms “about” and “approximately” are defined as being “'close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

As used herein the term “adjuvant” refers to a compound that, when used in combination with a specific immunogen in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

As used herein, the terms “antigen” or “immunogen” are used interchangeably to refer to a substance, typically a protein, a nucleic acid, a polysaccharide, a toxin, or a lipid, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

A “composition” is intended to include a combination of active agent and another compound or composition, inert (for example, a fusion protein, nucleic acid, or virus) or active, such as an adjuvant.

As used herein, the term “effective amount” refers to an amount of a composition necessary or sufficient to realize a desired biologic effect. An effective amount of the composition would be the amount that achieves a selected result, and such an amount could be determined as a matter of routine experimentation by a person skilled in the art. For example, an effective amount of the composition could be that amount necessary for preventing, treating and/or ameliorating viral infection and/or symptoms thereof in a subject. The term is also synonymous with “sufficient amount.” “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

An “immunological response” or “immunity” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein the term “protective immune response”, “protective response”, or “protective immunity” refers to an immune response mediated by antibodies against an infectious agent, which is exhibited by a vertebrate (e.g., a human), that prevents or ameliorates an infection or reduces at least one symptom thereof. The compositions of the invention can stimulate the production of antibodies that, for example, neutralize infectious agents, blocks infectious agents from entering cells, blocks replication of said infectious agents, and/or protect host cells from infection and destruction. The term can also refer to an immune response that is mediated by T cells, B cells, and/or other white blood cells against an infectious agent, exhibited by a vertebrate (e.g., a human), that prevents or ameliorates viral infection or reduces at least one symptom thereof.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In some embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “subject” is defined herein to include animals such as mammals, including, but not limited to, primates (e.g., humans), cows, sheep, goats, horses, dogs, cats, rabbits, rats, mice and the like. In some embodiments, the subject is a human. “Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

“Therapeutically effective amount” or “therapeutically effective dose” of a composition (e.g. a fusion protein, a nucleic acid, a vaccine) refers to an amount that is effective to achieve a desired therapeutic result. In some embodiments, a desired therapeutic result is the prevention of a viral infection or symptoms thereof. In some embodiments, a desired therapeutic result is the treatment of a viral infection or symptoms thereof. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as coughing relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, lentiviral vectors, adenoviral vectors, adeno-associated virus vectors, retroviral vectors, and the like.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi nlm nih gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=S, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=S, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

As used herein, the term “vaccine” refers to a formulation which contains the compositions (e.g., nucleic acids, polypeptides, or nanoparticles) of the present invention, which is in a form that is capable of being administered to a subject and which induces a protective immune response sufficient to induce immunity to prevent and/or ameliorate an infection and/or to reduce at least one symptom of an infection and/or to enhance the efficacy of another dose of the compositions (e.g., nucleic acids, polypeptides, or nanoparticles). Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of CD8+ T cells, antigen presenting cells, CD4+ T cells, dendritic cells and/or other cellular responses.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compositions

Disclosed herein is a platform for developing vaccines that can simultaneously present multiple and diverse antigens. This platform can lead to elicitation of immune responses with broad neutralization breath (i.e., neutralizing multiple variants/strains/clades/mutants of a pathogen). The vaccines disclosed herein can be DNA vaccines, RNA vaccines, protein vaccines, or nanoparticle vaccines.

In some aspects, disclosed herein is a recombinant nucleic acid comprising two or more polynucleotide sequences encoding two or more antigens, wherein the 3′ end of each of the two or more polynucleotide sequences encoding the two or more antigens is operably linked to a 2A polynucleotide sequence.

It should be understood that 2A peptides encoding the 2A polynucleotide sequence are short segments (˜20 amino acids in length) that promote ribosome skipping and therefore act as “self-cleaving” agents to result in multiple protein products from a single gene construct.

Accordingly, in some embodiments, the 2A polynucleotide sequence encodes a 2A polypeptide that is self-cleavage. In some embodiments, the 2A polynucleotide sequence is place between an antigen-coding polynucleotide sequence and a heterologous sequence (e.g., a polynucleotide sequence encoding a signal peptide, or a polynucleotide sequence encoding a ferritin protein). Accordingly, the recombinant nucleic acid sequence (e.g., a DNA sequence) is transcribed as a single transcript (e.g., an RNA sequence) and then translated to produce multiple polypeptides. In some embodiments, the 2A polynucleotide sequence comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 11 or 12. In some embodiments, the 2A polypeptide sequence comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 13 or 14.

In some embodiments, the 5′ end of each of the two or more polynucleotides encoding the two or more antigens is operably linked to a polynucleotide sequence encoding a signal peptide. The term “signal peptide” (sometimes referred to as signal sequence) herein refers to a peptide present at the N-terminus of a polypeptide that is destined toward the secretory pathway. Signal peptides can promote protein translocation to the cellular membrane. In some embodiments, the signal peptide described herein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 16. In some embodiments, the polynucleotide sequence encoding the signal peptide comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 15.

In some embodiments, the linker polynucleotide sequence herein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NOs: 25-28. In some embodiments, the linker polypeptide sequence comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 29 or 30.

The two or more antigens encoded by the recombinant nucleic acids disclosed herein can be any antigen, including, for example, antigens of pathogens or tumor antigens (e.g., tumor cell markers, tumor associated antigens, mutant/fusion proteins expressed by tumor cells). In some embodiments, the antigens are antigens of pathogens, including, for example, viral antigens, bacterial antigens, parasitic antigens, or fungal antigens. In some embodiments, the viral antigen can be an antigen of a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1,

Hantavirus, Rubella virus, Simian Immunodeficiency virus, Human Immunodeficiency virus type-1, and Human Immunodeficiency virus type-2.

In some embodiments, the two or more viral antigens are HIV antigens, influenza antigens, or coronavirus antigens (e.g., SARS-CoV2 antigens). The two or more viral antigens can be derived from same or different strains/variants/clades of a virus (e.g., HIV, influenza, or SARS-CoV2).

“HIV” refers to the human immunodeficiency virus. HIV includes, without limitation, HIV-1 and HIV-2. The HIV-1 virus may represent any of the known major subtypes or clades (e.g., Classes A, B, C, D, E, F, G, J, and H) or outlying subtype (Group 0). Also encompassed are other HIV-1 subtypes or clades that may be isolated. There are two distinct types of HIV, HIV-1 and HIV-2, which are distinguished by their genomic organization and their evolution from other lentiviruses. Based on phylogenetic criteria (i.e., diversity due to evolution), HIV-1 can be grouped into three groups (M, N, and O). Group M is subdivided into 11 clades (A through K). HIV-2 can be divided into six distinct phylogenetic lineages (clades A through F). HIV has an about 9.2kb unspliced genomic transcript which encodes for gag and pol precursors; a singly spliced, 4.5 kb encoding for env, Vif, Vpr and Vpu and a multiply spliced, 2 kb mRNA encoding for Tat, Rev and Nef. The recombinant nucleic acids disclosed herein can comprise two or more polynucleotide sequences encoding two or more HIV proteins, including, for examples, Gag proteins, Pol proteins, Env proteins, Tat proteins, Rev proteins, Nef proteins, Vpr proteins, Vif proteins, or Vpu proteins.

In some embodiments, the two or more HIV proteins comprise Env proteins. HIV Env protein is a trimeric, spike-shaped protein, with 3 identical molecules, each with a cap-like region called glycoprotein 120 (gp120) and a stem called glycoprotein 41 (gp41) that anchors

Env in the viral membrane. Env is synthesized as a heavily glycosylated gp160 protein and cleaved by the host furin protease to form a heterodimer (protomer) consisting of gp120 and gp41. Accordingly, in some embodiments, the two or more HIV proteins comprise a gp160 protein, a gp120 protein, a gp41 protein, or a fragment thereof. In some embodiments, the two or more HIV proteins are from the same or different strains/variants/clades of HIV. In some embodiments, the two or more clades of HIV comprise BG505, CZA97, 286.36, 5768.04, DU172.17, HT593.1, KNH1209.18, MB539.2B7, RHPA.7, RW020.2, or 5018.18. In some embodiments, the two or more clades of HIV comprise BG505 or CZA97. In some examples, the HIV Env protein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 6 or 8. In some examples, the recombinant nucleic acid disclosed herein comprise a polynucleotide encoding an HIV Env protein, wherein the polynucleotide comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 5 or 7.

In some embodiments, the HIV protein comprises a sequence at least 80% (at least about 80%, at least about 85%, at least about 90%, at least about 95%, at least about 96%, at least about 97%, at least about 98%, at least about 99%, or at least about 99.5%) identical to SEQ ID NO: 31, 35, 39, 43, 47, 51, 55, 59, or 63.

In some embodiments, the two or more HIV proteins comprise fusion peptides. The term “fusion peptide” refers to a fragment of HIV Env protein that is essential for mediating viral entry. A fusion comprising about 15 to about 20 hydrophobic residues at the N terminus of the Env-gp41 subunit. Elicitation of immune responses that block fusion peptide is key to inhibit HIV entry. It is shown herein that the immunogen described herein comprising a fusion peptide can be recognized by VRC34.01, an identified broadly neutralizing antibody of HIV. Accordingly, in some embodiments, the two or more HIV proteins comprise fusion peptides. In some examples, the recombinant nucleic acid disclosed herein comprise a polynucleotide encoding a fusion peptide, wherein the polynucleotide comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NOs: 17-19. In some examples, the recombinant nucleic acid disclosed herein comprise a polynucleotide encoding a fusion peptide, wherein the fusion peptide comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NOs: 20-24.

In some embodiments, the bacterial antigen can be antigen of a bacterium selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani,other Clostridium species, Yersinia enterolitica, and other Yersinia species.

In some embodiments, the parasitic antigen can be an antigen of a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

In some embodiments, the fungal antigen can be an antigen of a fungus selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneumocystis carnii, Penicillium marneffi, and Alternaria alternata.

In some embodiments, the polynucleotide sequence encoding the signal peptide, the polynucleotide sequence encoding the antigen, and the 2A polynucleotide sequence are operably linked.

In some embodiments, the recombinant nucleic acid disclosed herein further comprises a polynucleotide sequence encoding a ferritin protein. Ferritin is a blood protein that contains iron. Ferritin proteins can self-assemble into spherical nanoparticles and can serve as a scaffold to express a heterologous protein, such as viral proteins, so it mimics a physiologically relevant viral spike. In some embodiments, the ferritin-based nanoparticle presents viral proteins on its surface. In some embodiments, the polynucleotide sequence encoding the ferritin protein is operably linked to the 3′ end of each of the two or more of the polynucleotide sequences encoding the two or more antigens and to the 5′ end of the 2A polynucleotide sequence. In some embodiments, the ferritin protein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 10. In some embodiments, the polynucleotide sequence encoding the ferritin protein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 9.

In some embodiments, the recombinant nucleic acid disclosed herein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 1 or 3. In some embodiments, the recombinant nucleic acid disclosed herein encodes a polypeptide sequence at least about 80% (at least about 80%, at least about 85%, at least about 90%, at least about 95%, at least about 96%, at least about 97%, at least about 98%, at least about 99%, or at least about 99.5%) identical to SEQ ID NO: 2, 4, 32-34, 36-38, 40-42, 44-50, 52-54, 56-58, or 60-62.

In some aspects, disclosed herein is a DNA vaccine comprising the recombinant nucleic acid disclosed herein.

As used in this disclosure, the term “DNA vaccine” comprises DNA sequences that code for immunogenic proteins located in appropriately constructed plasmids, which include a promoter, which when injected into an animal are taken up by cells and the immunogenic proteins are expressed and elicit an immune response. DNA vaccines are known in the art. See, e.g., U.S. Pat. No.: 8,535,687, and U.S. Patent Application Publication NOs: 2019/0112351 and 2007/0253969 incorporated by reference herein in their entireties.

In some aspects, disclosed herein is an RNA vaccine comprising a sequence that is transcribed from the recombinant nucleic acid disclosed herein. Methods for producing RNA vaccines are known in the art. See, e.g., U.S. Pat. Nos.: 10,485,884 and 9,295,717, and U.S. Patent Application Publication No: 20170136121, incorporated by reference herein in their entireties.

In some aspects, disclosed herein is a protein vaccine comprising two or more polypeptides that are transcribed from the recombinant nucleic acid disclosed herein. In some embodiments, the protein vaccine comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 2, 4, 32-34, 36-38, 40-42, 44-50, 52-54, 56-58, 60-62, or a fragment thereof. In some embodiments, the two or more polypeptides that are transcribed from the recombinant nucleic acid comprise a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 6, 8, 31, 35, 39, 43, 47, 51, 55, 59, 63, or a fragment thereof.

In some embodiments, the DNA vaccine, the RNA vaccine, or the protein vaccine described herein further comprises a pharmaceutically acceptable carrier. In some embodiments, the DNA vaccine, the RNA vaccine, or the vaccine comprising one or more polypeptides described herein described herein is formulated inside a nanoparticle.

As used herein, the term “nanoparticle” refers to any particle having a diameter making the particle suitable for systemic, in particular parenteral, administration, of, in particular, nucleic acids, typically a diameter of less than about 1000 nanometers (nm). In some embodiments, a nanoparticle has a diameter of less than about 600 nm (including, for example, less than about 500 nm, less than about 400 nm, less than about 300 nm, less than about 200 nm, less than about 100 nm, less than about 50 nm, less than about 20 nm, or less than about 10 nm). In some embodiments, the nucleic acids or polypeptides disclosed herein are encapsulated inside a nanoparticle. In some embodiments, the nucleic acids or polypeptides disclosed herein are embedded in the membrane of a nanoparticle. In some embodiments, the nucleic acids or polypeptides disclosed herein are present on the surface of a nanoparticle.

As used herein, the term “nanoparticulate formulation” or similar terms refer to any substance that contains at least one nanoparticle. In some embodiments, a nanoparticulate composition is a uniform collection of nanoparticles. In some embodiments, nanoparticulate compositions are dispersions or emulsions. In general, a dispersion or emulsion is formed when at least two immiscible materials are combined.

Also disclosed herein is a recombinant nucleic acid comprising a polynucleotide sequence encoding an antigen, wherein the 3′ end of each of the polynucleotide sequence encoding the antigen is operably linked to a polynucleotide sequence encoding a ferritin protein.

In some embodiments, the ferritin protein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 10. In some embodiments, the polynucleotide sequence encoding the ferritin protein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 9.

In some embodiments, the antigen is a viral antigen disclosed herein, including, for example, an HIV antigen, an influenza antigen, or a SARS-CoV-2 antigen. In some embodiments, the HIV antigen is an Env. In some embodiments, the HIV antigen comprises a gp160 protein, a gp120 protein, a gp41 protein, or a fragment thereof. In some embodiments, the HIV antigen comprises a fusion peptide. In some embodiments, the HIV antigen is derived from BG505 or CZA97. In some embodiments, the HIV antigen comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 6 or 8. In some embodiments, the polynucleotide sequence encoding the HIV antigen comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 5 or 7.

Also disclosed herein is a recombinant nucleic acid comprising two or more polynucleotide sequences encoding two or more antigens, wherein the 3′ end of each of the two or more polynucleotide sequences encoding the two or more antigens is operably linked to a polynucleotide sequence encoding a ferritin protein and a 2A polynucleotide sequence.

In some embodiments, the 2A polynucleotide sequence encodes a 2A polypeptide that is self-cleavage. In some embodiments, the 2A polypeptide sequence comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 13 or 14. In some embodiments, the 2A polynucleotide sequence comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 11 or 12.

In some embodiments, the polynucleotide sequence encoding the ferritin protein is operably linked to the 3′ end of each of the two or more of the polynucleotide sequences encoding the two or more antigens and to the 5′ end of the 2A polynucleotide sequence.

In some embodiments, the recombinant nucleic acid further comprises a polynucleotide sequence encoding a signal peptide. In some embodiments, the signal peptide described herein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 16. In some embodiments, the polynucleotide sequence encoding the signal peptide comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 15.

In some embodiments, the linker polynucleotide sequence herein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NOs: 25-28. In some embodiments, the linker polypeptide sequence comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 29 or 30.

The two or more antigens encoded by the recombinant nucleic acids disclosed herein can be any antigen, including, for example, antigens of pathogens or tumor antigens (e.g., tumor cell markers, tumor associated antigens, mutant/fusion proteins expressed by tumor cells). In some embodiments, the antigens are antigens of pathogens, including, for example, viral antigens, bacterial antigens, parasitic antigens, or fungal antigens.

In some embodiments, the antigens are viral antigens disclosed herein. In some embodiments, the viral antigens are HIV antigens, influenza antigens, or SARS-CoV-2 antigens. The two or more viral antigens can be derived from same or different strains/variants/clades of a virus (e.g., HIV, influenza, or SARS-CoV2). In some embodiments, the two or more HIV proteins comprise Env proteins. In some embodiments, the two or more HIV proteins comprise a gp160 protein, a gp120 protein, a gp41 protein, or a fragment thereof. In some embodiments, the two or more HIV proteins comprise fusion peptides. In some embodiments, the two or more HIV proteins are from same or different strains/variants/clades of HIV. In some embodiments, the two or more clades of HIV comprise BG505, CZA97, 286.36, 5768.04, DU172.17, HT593.1, KNH1209.18, MB539.2B7, RHPA.7, RW020.2, or S018.18. In some embodiments, the two or more clades of HIV comprise BG505 or CZA97. In some embodiments, the polynucleotide sequence encoding the HIV antigen comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 6 or 8. In some embodiments, the polynucleotide sequence encoding the HIV antigen comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 5 or 7.

In some embodiments, the HIV protein comprises a sequence at least 80% (at least about 80%, at least about 85%, at least about 90%, at least about 95%, at least about 96%, at least about 97%, at least about 98%, at least about 99%, or at least about 99.5%) identical to

SEQ ID NO: 31, 35, 39, 43, 47, 51, 55, 59, or 63.

In some examples, the recombinant nucleic acid disclosed herein comprise a polynucleotide encoding a fusion peptide, wherein the polynucleotide comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NOs: 17-19. In some examples, the recombinant nucleic acid disclosed herein comprise a polynucleotide encoding a fusion peptide, wherein the fusion peptide comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NOs: 20-24.

In some embodiments, the recombinant nucleic acid disclosed herein comprises a sequence at least about 80% (at least about 80%, at least about 85%, at least about 90%, at least about 95%, at least about 96%, at least about 97%, at least about 98%, at least about 99%, or at least about 99.5%) identical to SEQ ID NO: 1. In some embodiments, the recombinant nucleic acid disclosed herein encodes a polypeptide sequence at least about 80% (at least about 80%, at least about 85%, at least about 90%, at least about 95%, at least about 96%, at least about 97%, at least about 98%, at least about 99%, or at least about 99.5%) identical to SEQ ID NO: 2, 32-34, 36-38, 40-42, 44-50, 52-54, 56-58, or 60-62.

As discussed above, Ferritin proteins can self-assemble into spherical nanoparticles and can serve as a scaffold to express a heterologous protein, such as viral proteins, so it mimics a physiologically relevant viral spike. In some embodiments, the ferritin-based nanoparticle presents the viral proteins disclosed herein (e.g., an HIV Env protein) on its surface. Accordingly, in some aspects, disclosed herein is a nanoparticle vaccine encoded by the recombinant nucleic acid disclosed herein, wherein the recombinant nucleic acid comprises two or more polynucleotide sequences encoding two or more antigens, wherein the 3′ end of each of the two or more polynucleotide sequences encoding the two or more antigens is operably linked to a polynucleotide sequence encoding a ferritin protein and a 2A polynucleotide sequence.

Optionally, the vaccine contemplated herein can be combined with an adjuvant such as Freund's incomplete adjuvant, Freund's Complete adjuvant, alum, monophosphoryl lipid A, alum phosphate or hydroxide, QS-21, salts, i.e., A1K(SO4)2, AlNa(SO4)2, A1NH4(SO4)2, silica, kaolin, carbon polynucleotides, i.e., poly IC and poly AU. Additional adjuvants can include QuilA and Alhydrogel and the like. Optionally, the vaccine contemplated herein can be combined with immunomodulators and immunostimulants such as interleukins, interferons and the like. Many vaccine formulations are known to those of skill in the art.

In some embodiments, the vaccine further comprises a pharmaceutically acceptable carrier.

To promote intracellular introduction of an expression vector, the therapeutic or improving agent of the present invention may further contain a reagent for nucleic acid introduction. As the reagent for nucleic acid introduction, cationic lipids such as lipofectin (trade name, Invitrogen), lipofectamine (trade name, Invitrogen), transfectam (trade name, Promega), DOTAP (trade name, Roche Applied Science), dioctadecylamidoglycyl spermine (DOGS), L-dioleoyl phosphatidyl-ethanolamine (DOPE), dimethyldioctadecyl-ammonium bromide (DDAB), N,N-di-n-hexadecyl-N,N-dihydroxyethylammonium bromide (DHDEAB), N-n-hexadecyl-N,N-dihydroxyethylammonium bromide (HDEAB), polybrene, poly(ethyleneimine) (PEI) and the like can be used. In addition, an expression vector may be included in any known liposome constituted of a lipid bilayer such as electrostatic liposome. Such liposome may be fused with a virus such as inactivated Hemagglutinating Virus of Japan

(HVJ). HVJ-liposome has a very high fusion activity with a cellular membrane, as compared to general liposomes. When retrovirus is used as an expression vector, RetroNectin, fibronectin, polybrene and the like can be used as transfection reagents.

### Methods of Treating or Preventing Infection

In some aspects, disclosed herein is a method of treating and/or preventing an infection in a subject, comprising administering to the subject an effective amount of the DNA vaccine disclosed herein. In some embodiments, the DNA vaccine comprises the recombinant nucleic acid disclosed herein that comprises two or more polynucleotide sequences encoding two or more antigens, wherein the 3′ end of each of the two or more polynucleotide sequences encoding the two or more antigens is operably linked to a 2A polynucleotide sequence.

In some aspects, disclosed herein is a method of treating and/or preventing an infection in a subject, comprising administering to the subject an effective amount of the RNA vaccine disclosed herein. In some embodiments, the RNA vaccine comprises a sequence transcribed from the recombinant nucleic acid disclosed herein that comprises two or more polynucleotide sequences encoding two or more antigens, wherein the 3′ end of each of the two or more polynucleotide sequences encoding the two or more antigens is operably linked to a 2A polynucleotide sequence.

In some aspects, disclosed herein is a method of treating and/or preventing an infection in a subject, comprising administering to the subject an effective amount of the protein vaccine disclosed herein. In some embodiments, the protein vaccine comprises two or more polypeptides that are transcribed from the recombinant nucleic acid disclosed herein, wherein the recombinant nucleic acid comprises two or more polynucleotide sequences encoding two or more antigens, wherein the 3′ end of each of the two or more polynucleotide sequences encoding the two or more antigens is operably linked to a 2A polynucleotide sequence.

In some aspects, disclosed herein is a method of treating and/or preventing an infection in a subject, comprising administering to the subject an effective amount of the nanoparticle vaccine disclosed herein. In some embodiments, the nanoparticle vaccine is encoded by the recombinant nucleic acid disclosed herein, wherein the recombinant nucleic acid comprises two or more polynucleotide sequences encoding two or more antigens, wherein the 3′ end of each of the two or more polynucleotide sequences encoding the two or more antigens is operably linked to a polynucleotide sequence encoding a ferritin protein and a 2A polynucleotide sequence.

In some embodiments, the infection can be an infection of a virus, a bacterium, a parasite, or a fungus.

In some embodiments, the infection can be an infection of a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1,

Hantavirus, Rubella virus, Simian Immunodeficiency virus, Human Immunodeficiency virus type-1, and Human Immunodeficiency virus type-2.

In some embodiments, the infection can be infection of a bacterium selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica,and other Yersinia species.

In some embodiments, the infection can be an infection of a parasite selected from the group consisting of Toxoplasma gondii, Plasmodium falciparum, Plasmodium vivax,

Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

In some embodiments, the infection can be an infection of a fungus selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneumocystis carnii, Penicillium marneffi, and Alternaria alternata.

In some embodiments, the infection is HIV infection. In some embodiments, the infection is SARS-CoV-2 infection. In some embodiments, the infection is influenza infection.

The vaccines of the present invention can be administered to the appropriate subject in any manner known in the art, e.g., orally intramuscularly, intravenously, sublingual mucosal, intraarterially, intrathecally, intradermally, intraperitoneally, intranasally, intrapulmonarily, intraocularly, intravaginally, intrarectally or subcutaneously. They can be introduced into the gastrointestinal tract or the respiratory tract, e.g., by inhalation of a solution or powder containing the conjugates. In some embodiments, the compositions can be administered via absorption via a skin patch. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant level of dosage is maintained. In some embodiments, the one or more effective doses of the vaccine are administered to the subject via a route that is selected from the group consisting of an intramuscular route, a subcutaneous route, an intradermal route, an oral administration, a nasal administration, and inhalation.

A pharmaceutical composition (e.g., a vaccine) is administered in an amount sufficient to elicit production of antibodies and activation of CD4+ T cells and CD8+ T cells as part of an immunogenic response. Dosage for any given patient depends upon many factors, including the patient's size, general health, sex, body surface area, age, the particular compound to be administered, time and route of administration, and other drugs being administered concurrently. Determination of optimal dosage is well within the abilities of a pharmacologist of ordinary skill.

The method comprises administering to the recipient one or more than one dose of a vaccine according to the present invention. In a preferred embodiment, the vaccine is administered in a plurality of doses. In another preferred embodiment, the dose is between about 0.001 mg/kg of body weight of the recipient and about 1000 mg/kg of body weight of the recipient. In another preferred embodiment, the dose is between about 0.001 mg/kg of body weight of the recipient and about 100 mg/kg of body weight of the recipient. In another preferred embodiment, the dose is between about 0.01 mg/kg of body weight of the recipient and about 10 mg/kg of body weight of the recipient. In another preferred embodiment, the dose is between about 0.1 mg/kg of body weight of the recipient and about 1 mg/kg of body weight of the recipient. In another preferred embodiment, the dose is about 0.05 mg/kg of body weight of the recipient. In a preferred embodiment, the recipient is a human and the dose is between about 0.5 mg and 5 mg. In another preferred embodiment, the recipient is a human and the dose is between about 1 mg and 4 mg. In another preferred embodiment, the recipient is a human and the dose is between about 2.5 mg and 3 mg. In another preferred embodiment, the dose is administered weekly between 2 times and about 100 times. In another preferred embodiment, the dose is administered weekly between 2 times and about 20 times. In another preferred embodiment, the dose is administered weekly between 2 times and about 10 times. In another preferred embodiment, the dose is administered weekly 4 times. In another preferred embodiment, the dose is administered once, 2 times, 3 times, or 4 times. In some embodiments, any combination of any 2, 3, 4, etc. strains from the set of 9 strains (286.36, 5768.04, DU172.17, HT593.1, KNH1209.18, MB539.2B7, RHPA.7, RW020.2, and 5018.18) can be combined in the 2A and insect ferritin 2A format.

### EXAMPLES

The following examples are set forth below to illustrate the compounds, systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

### Design and Development of Empirical and Rational Epitope-Focused HIV-1 Vaccines

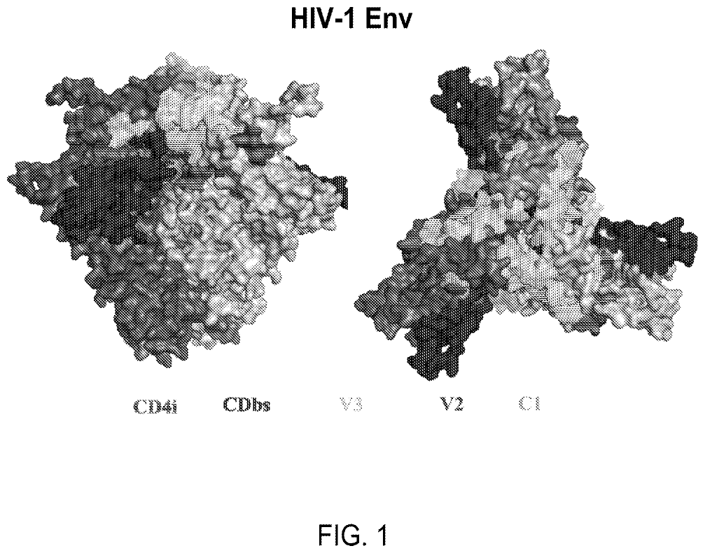

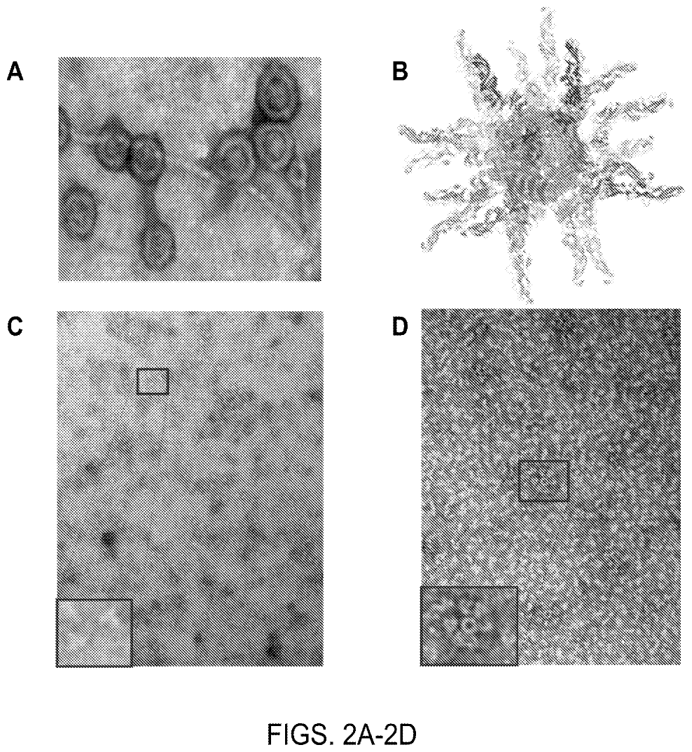

Nanoparticle immunogens were developed to simultaneously present 1) multiple, diverse Envs or 2) relatively conserved domains of the envelope protein to the immune system. The present example shows the design, development, and validation of a number of these technologies (FIGS. 1-2). FIG. 1 shows structures of HIV-1 Env by common epitopes. FIGS. 2A-2D show the vaccine platforms. FIG. 2A shows analysis of nanoparticles from phage MS2 capsid. Negative-stain EM shows the formation of particles of the expected size. FIG. 2B shows structural model of an antigen (colored spikes) on a ferritin particle (green). The antigen can be fused to either the N- or C-terminus of the particle protein. FIG. 2C shows successful expression and purification of HIV-1 Env trimers which are used as cocktail immunogens in animal studies. FIG. 2D shows expression of ferritin nanoparticle immunogens mounted with HIV-1 Env proteins.

### Example 2

### Vaccines Using Different Clades

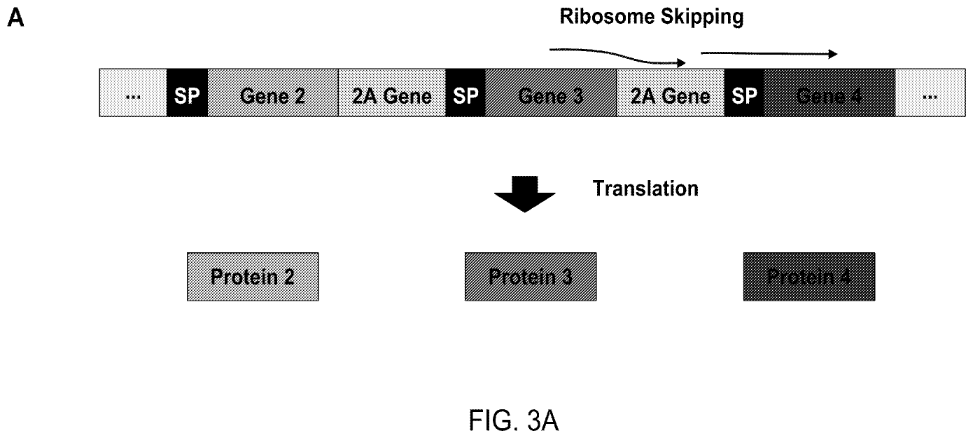

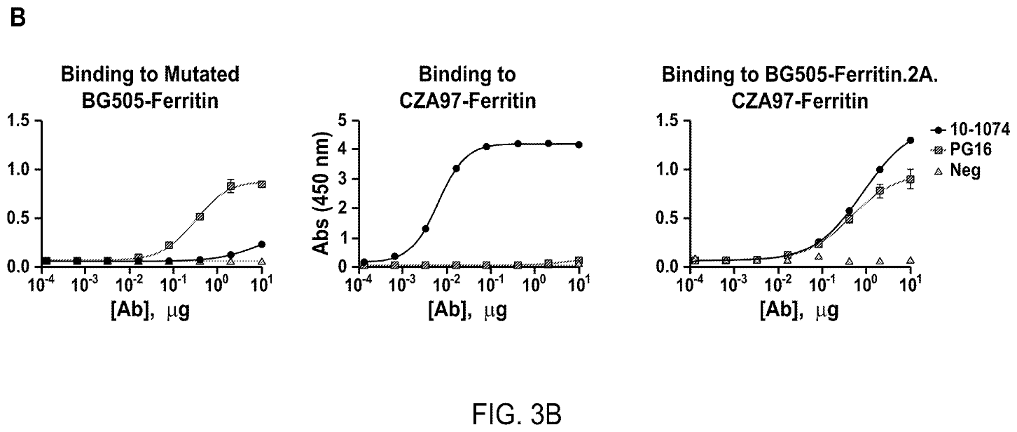

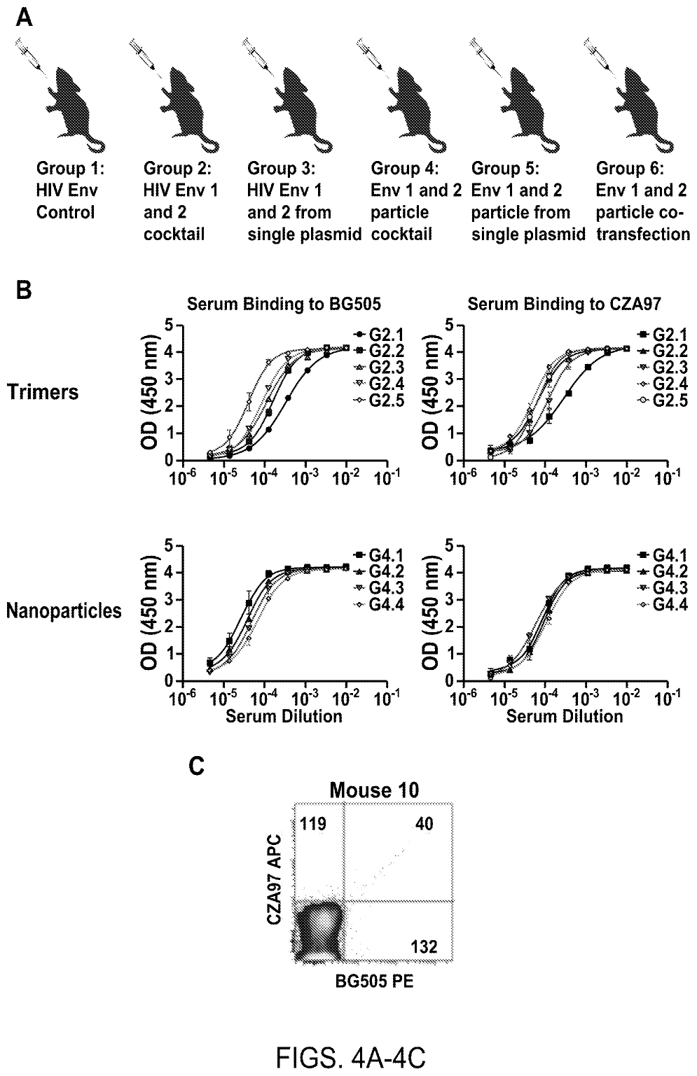

Vaccines displaying Envs from two different clades have been successfully designed and developed (FIGS. 3-4). FIGS. 3A-3B show 2A peptide generated antigens. FIG. 3A shows schematic of multiantigen DNA using 2A peptides as separators between the different antigen genes. 2A peptides are typically short segments (˜20 amino acids in length) that promote ribosome skipping and therefore act as “self-cleaving” agents to result in multiple protein products from a single gene construct. This technology can be implemented in delivering a DNA vaccine. FIG. 3B shows ELISAs validating expression of multiple Envs from a single transcript. Antibodies specific to each Env trimer variant were used to identify expression of each Env. FIGS. 4A-4C show animal studies. FIG. 4A shows immunization groups. Trimer cocktails, nanoparticle cocktails and co-expressed nanoparticles were used to intramuscularly immunize BALB/c mice. Mice were exsanguinated at day 70 for serological analyses. FIG. 4B shows immunizations with nanoparticles elicit comparable antibody titers when compared to titers elicited in response to immunizations with trimer cocktails. FIG. 4C shows antigen specific B-cell sorting shows B-cells that are cross-reactive to the two trimers in the vaccines.

### Example 3

### Neutralization in Mice

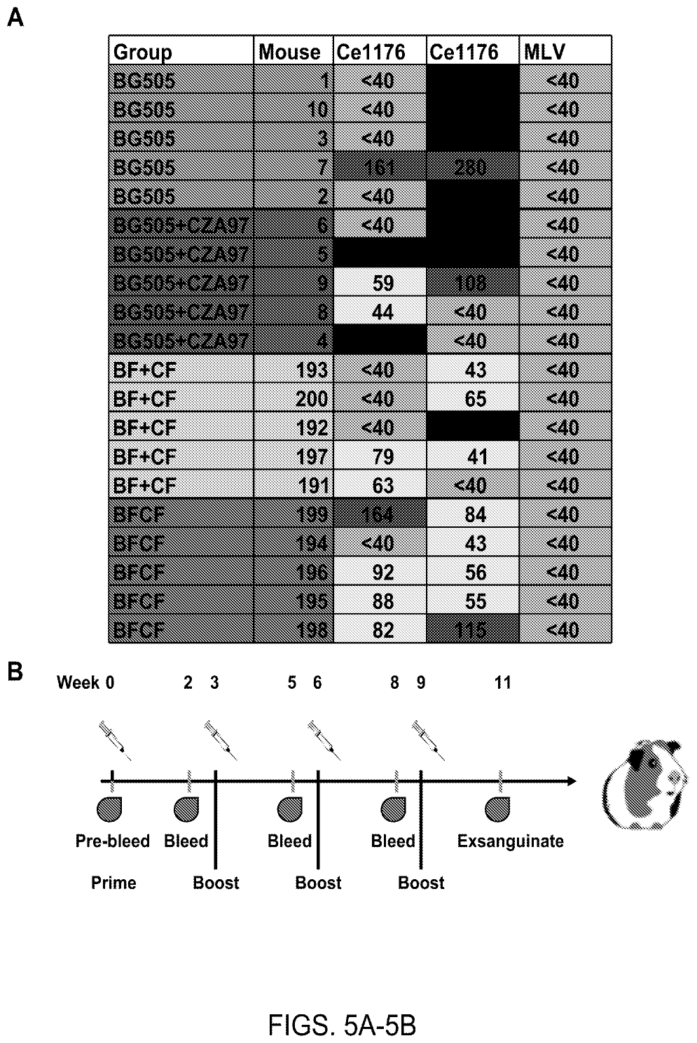

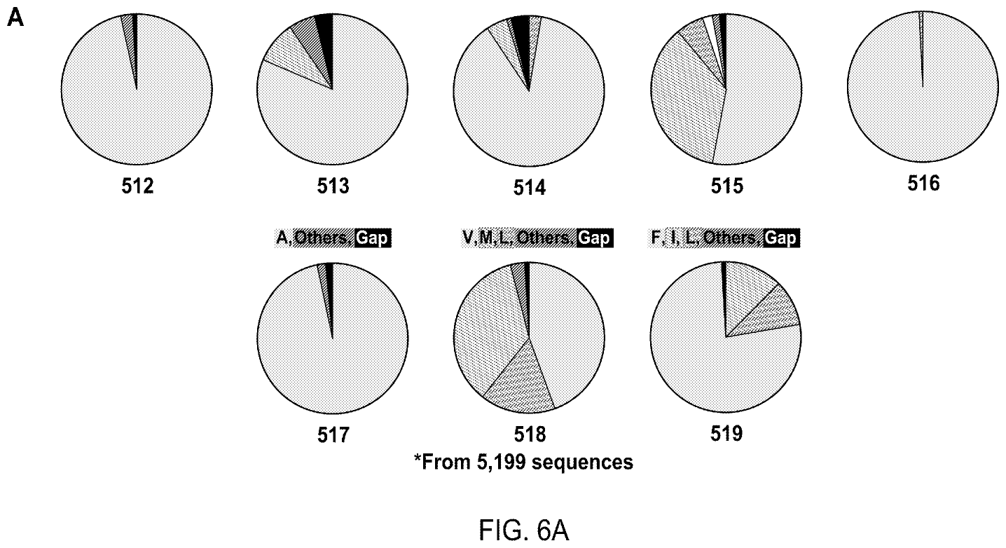

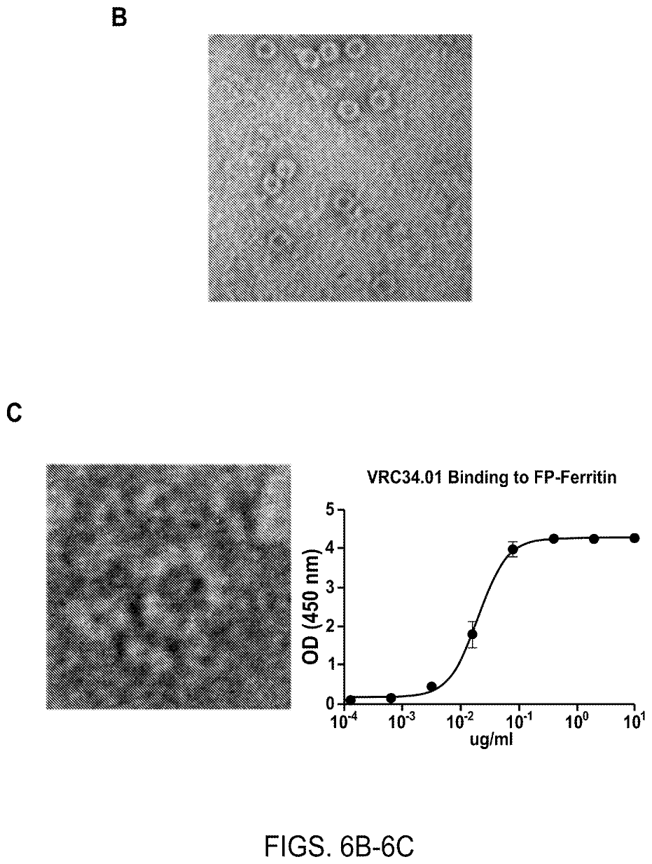

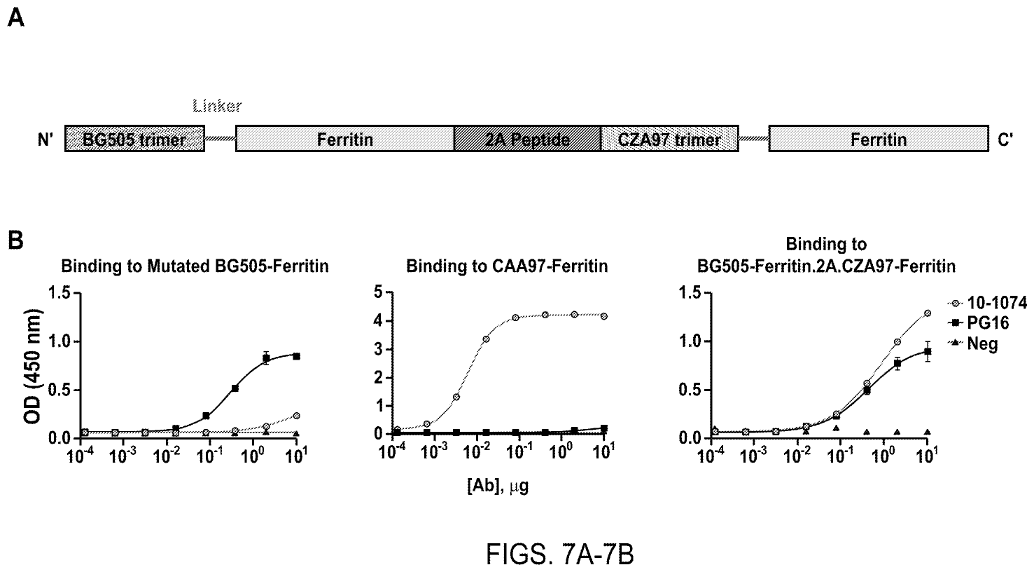

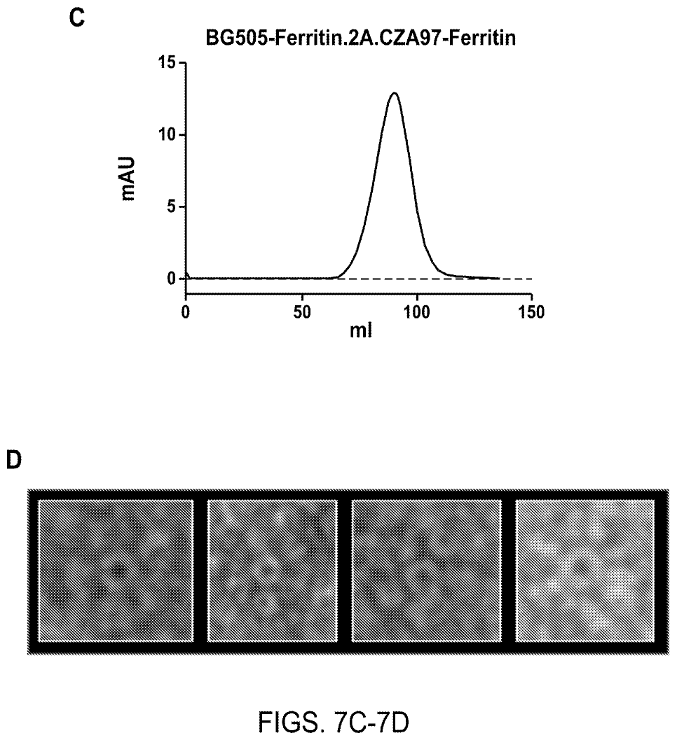

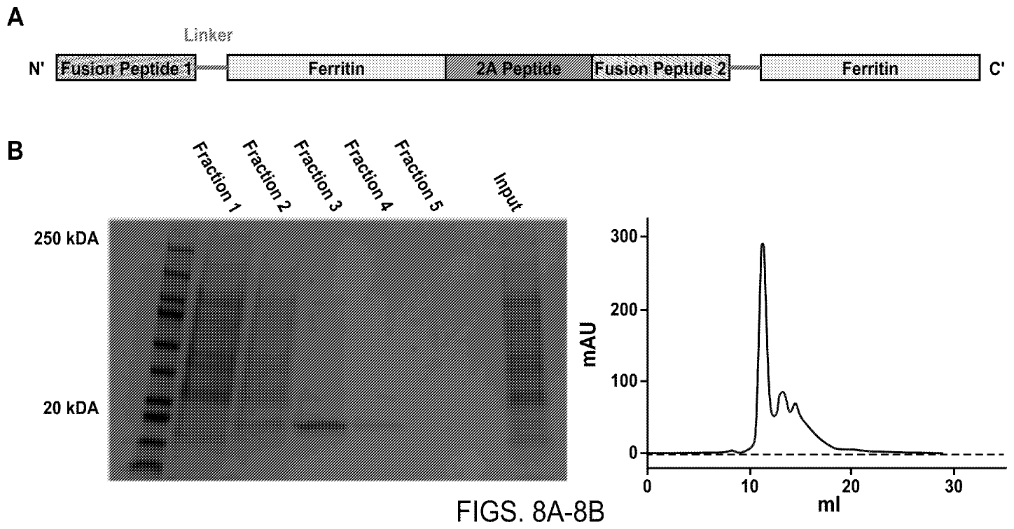

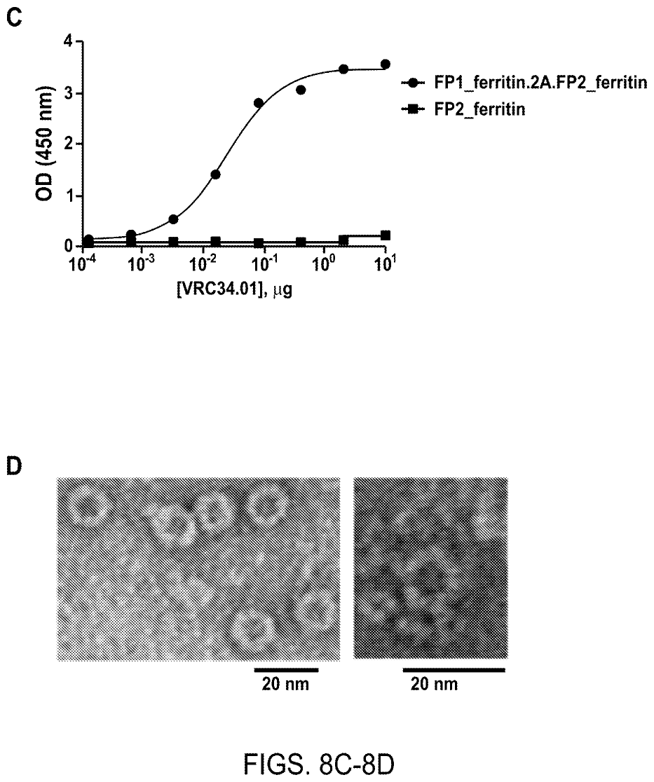

The vaccines show heterologous neutralization in mice (FIG. 5A). Further, nanoparticles bearing the fusion peptide of HIV were expressed, purified and characterized and are tested in guinea pigs (FIG. 5B and FIG. 6). FIGS. 5A-5B show a study indicating heterologous breadth. FIG. 5A shows mouse sera showing neutralization against a heterologous Tier 2 virus, Ce1176. FIG. 5B shows that nanoparticles were used to immunize guinea pigs. FIGS. 6A-6C show expression and characterization of fusion-peptide nanoparticle vaccines. FIG. 6A shows the fusion peptide of HIV-1 is relatively conserved. Selection of fusion peptides should incorporate maximum diversity in order to cover the majority of circulating strains. FIG. 6B shows successful expression of fusion-peptide-ferritin is evident from negative-stain EM. FIG. 6C shows that fusion peptide nanoparticles are recognized by monoclonal antibody VRC34.01 as evidenced by negative-stain EM and ELISA. This antibody binds to the fusion peptide of HIV-1.

When compared to soluble trimer cocktails and BG505 alone, BG505 nanoparticle and CZA97 nanoparticle cocktails as well as nanoparticles bearing both elicit better responses and show heterologous neutralization in mice. Binding results show the nanoparticle constructs elicit antibody responses in guinea pigs as well.

While efficacious for HIV-vaccines, the technologies and vaccine platforms described herein can also be used for vaccine design for other viruses that exhibit high levels of sequence diversity.

### Example 4

### HIV Strain Selection

A search for optimal combinations of six strains was performed. A multi-optimization algorithm was applied to identify sets of size six based on glycan shield coverage, neutralization sensitivity, and sequence diversity.

Specifically, the goal was to identify sets of strains with:

(i) High glycan shield coverage. “Glycan holes”, corresponding to missing conserved glycans in a strain, have been implicated in eliciting autologous neutralizing antibodies that are not capable of developing neutralization breadth since these glycan holes are not present in the majority of other strains. Hence, the goal was to select combinations of strains that minimize the existence of shared glycan holes.

(ii) High bNAb neutralization sensitivity. Strains that are potently neutralized by the majority of bNAb specificities were selected, therefore giving the immune system the opportunity to recognize an epitope from a larger set of possibilities; this is in contrast to strains that may only present a limited set of bNAb epitopes, which is a strategy that can be used in epitope-focused vaccine development; rather, the goal here was to increase the chances of recognizing any bNAb epitope, as opposed to a specific bNAb epitope.

(iii) Env sequence diversity. Computational modeling has suggested that optimal sequence diversity within a multivalent vaccine may promote the ability to elicit neutralization breadth. The optimization algorithm therefore considered several different scenarios: low/intermediate/high sequence diversity within a single clade/two clades/all clades.

(iv) Number of strains in a combination. The number of strains used in a multivalent vaccine may have opposing effects: on the one hand, adding more strains may allow for closer mimicking of virus swarms during HIV-1 infection; on the other hand, the inclusion of more strains may increase the likelihood of generating off-target antibody responses; further, the clinical-grade production of a greater number of constructs may pose substantial challenges. Optimizing the number of strains used as part of multivalent vaccines is therefore of significance. To that end, the search algorithm was applied for sets of strains of different size, ranging from 4 to 10 strains, and identifying optimal sets (with respect to the glycan shield, neutralization sensitivity, and sequence diversity variables) for each size. This analysis helped identify set sizes that balance between optimal properties and number of strains included. Next, details are provided for the different variables that were evaluated in this optimization approach.

Glycan shield coverage: A set of ˜5,000 representative HIV-1 strains was selected from the LANL HIV database and their Env proteins were aligned to the reference HXB2 strain. From the Env alignment, all residue positions that correspond to an N-linked glycosylation sequon [74] were extracted from each strain. Residue positions for which at least x% of strains had an N-linked glycosylation sequon were defined as conserved glycan positions. The initial percentage was set at x=50%, requiring at least half of the representative strains to have a glycan for a given residue position to be considered conserved. Then, for each given strain, the fraction of residue positions that have a glycan at the conserved glycan positions was computed. For residue positions from the conserved glycan set that do not have a glycan in a given strain, structural analysis of the Env trimer structure was performed to identify potential compensatory glycans that are within 10Å of a missing conserved glycan. For each strain, the list of conserved and compensatory glycans was then used for further analysis.

Availability of bNAb epitopes: Published datasets of bNAb-virus were compiled. bNAbs were divided into a discrete set of epitope specificities. For each strain and bNAb specificity group, the minimum (best), median, and maximum (worst) neutralization IC₅₀ values among all bNAbs in that group were computed. Strains with minimum neutralization values of greater than 1 μg/ml for any bNAb specificity group, and strains for which two or more bNAb groups included an antibody that cannot neutralize the given strain (typically, an IC₅₀ value of >50 μg/ml) were filtered out. In addition, strains that are sensitive to weakly/non-neutralizing antibodies (such as F105, 17b, etc.) were also filtered out. The remaining strains were used for further optimization.

Env sequence diversity: The Env sequence diversity within each combination of strains was computed. This was done both for the entire Env SOSIP sequence (to account for overall clade diversity), as well as specifically for the protein surface residue positions (to account for antibody epitope diversity).

Finally, a number of strain sets were identified that had high glycan shield coverage, high bNAb neutralization sensitivity, and high sequence diversity.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

SEQUENCES
SEQ ID NO: 1
DNA sequence of
BG505.SOSIP.664scLinkerFerritin_2A_
CZA97.SOSIP.664scLinkerFerritin
atgcccatgggcagcctgcagcccctggccaccctgtacctgctg
ggcatgctggtggctagcgtgctggccgccgaaaacctgtgggtc
accgtgtattatggagtgcccgtctggaaagatgctgaaactacc
ctgttctgtgcctctgatgctaaggcctacgagaccgaaaagcac
aatgtctgggctactcatgcatgcgtgcccaccgacccaaacccc
caggagatccacctggaaaatgtgaccgaggaattcaacatgtgg
aaaaacaatatggtggagcagatgcatacagacatcattagcctg
tgggatcagtccctgaagccctgcgtcaaactgactcctctgtgc
gtgaccctgcagtgtaccaatgtcacaaacaatatcaccgacgat
atgaggggcgagctgaagaattgtagcttcaacatgaccacagaa
ctgagagacaagaaacagaaagtgtactccctgttttataggctg
gatgtggtccagatcaatgagaaccaggggaatcggagcaacaat
tccaacaaggaatacagactgatcaattgcaacacttccgccatt
acccaggcttgtcctaaagtgtcttttgagcctatcccaattcat
tattgcgccccagctggcttcgccatcctgaagtgtaaagataag
aagttcaacggaactggcccctgcccttccgtgtctacagtccag
tgtactcacgggattaagcctgtggtctctacacagctgctgctg
aatggaagtctggctgaggaagaagtgatgatccggagcgagaac
attaccaacaatgccaagaatatcctggtccagttcaacacacca
gtgcagattaattgcacaagacccaacaataacactcgaaaatct
atccggattgggccaggacaggccttttacgctacaggggacatc
attggagatatcagacaggctcactgtaCCgtgagtaaggcaacc
tggaacgagacactgggcaaggtggtcaaacagctgaggaaacat
ttcgggaataacaccatcattcgctttgccaatagctccggaggg
gacctggaggtcactacccactccttcaactgcggaggcgaattc
ttttactgtaacacatctggcctgtttaatagtacatggatctct
aacactagtgtgcagggcagtaattcaactgggtcaaacgatagc
atcaccctgccatgccgaattaagcagatcattaatatgtggcag
cggatcggccaggcaatgtatgccccccctatccagggggtcatt
cgctgcgtgagcaatatcaccggactgattctgacacgagacggg
ggcagcaccaactctacaactgaaacattccggcccggcggggga
gacatgagagataactggaggtccgagctgtacaagtataaagtg
gtcaagatcgaacctctgggagtggcaccaaccagatgcaagcga
agagtggtcggaGGCGGCAGCGGCGGCGGCGGCTCCGGCGGCGGC
GGCTCTGGCGGCgcagtcggaattggggccgtgttcctgggattt
ctgggcgccgctgggagtacaatgggagcagcctcaatgactctg
accgtgcaggccaggaatctgctgagcggcatcgtccagcagcag
tccaacctgctgcgcgctcctgaagcacagcagcacctgctgaag
ctgaccgtgtggggcatcaaacagctgcaggctagggtgctggca
gtcgagcggtacctgagagaccagcagctgctgggaatctggggc
tgctctgggaagctgatttgttgcacaaatgtgccttggaactct
agttggtcaaatcgcaacctgagcgagatctgggacaatatgact
tggctgcagtgggataaagaaattagtaactacacccagatcatc
tacggcctgctggaagagtcacagaatcagcaggagaagaacgaa
caggacctgctggcactggatGGCAGCGGCGATATCATCAAGCTG
CTGAACGAGCAAGTGAATAAGGAGATGCAGAGCTCCAACCTGTAC
ATGAGCATGTCTAGCTGGTGCTATACCCACTCCCTGGACGGAGCA
GGACTGTTCCTGTTTGATCACGCCGCCGAGGAGTATGAGCACGCC
AAGAAGCTGATCATCTTTCTGAATGAGAACAATGTGCCCGTGCAG
CTGACCTCCATCTCTGCCCCTGAGCACAAGTTCGAGGGCCTGACA
CAGATCTTTCAGAAGGCCTACGAGCACGAGCAGCACATCAGCGAG
TCCATCAACAATATCGTGGACCACGCCATCAAGTCCAAGGATCAC
GCCACATTCAACTTTCTGCAGTGGTACGTGGCCGAGCAGCACGAG
GAGGAGGTGCTGTTCAAGGACATCCTGGATAAGATCGAGCTGATC
GGCAACGAGAATCACGGCCTGTACCTGGCCGACCAGTATGTGAAG
GGCATCGCCAAGTCTCGGAAGAGCGgaagcggagctactaacttc
agcctgctgaagcaggctggagacgtggaggagaaccctggacct
ggaagcggaAtgcccatgggcagcctgcagcccctggccaccctg
tacctgctgggcatgctggtggctagcgtgctggccGTGGGCAAC
ATGTGGGTGACAGTGTACTATGGCGTGCCCGTGTGGACCGATGCC
AAGACCACACTGTTCTGCGCCTCCGACACAAAGGCCTACGATCGG
GAGGTGCACAACGTGTGGGCAACACACGCATGCGTGCCAACCGAC
CCAAATCCCCAGGAGATCGTGCTGGAGAACGTGACCGAGAACTTC
AACATGTGGAAGAACGACATGGTGGATCAGATGCACGAGGACATC
ATCAGCCTGTGGGATCAGTCCCTGAAGCCATGCGTGAAGCTGACA
CCCCTGTGCGTGACCCTGCACTGTACAAACGCCACCTTTAAGAAC
AATGTGACCAATGATATGAACAAGGAGATCAGGAATTGTTCTTTC
AACACCACAACCGAGATCCGCGATAAGAAGCAGCAGGGCTACGCC
CTGTTTTATAGGCCTGACATCGTGCTGCTGAAGGAGAATCGCAAC
AATTCTAACAATAGCGAGTATATCCTGATCAATTGCAACGCCAGC
ACAATCACCCAGGCCTGTCCCAAGGTGAACTTCGACCCTATCCCA
ATCCACTACTGCGCCCCTGCCGGCTATGCCATCCTGAAGTGTAAC
AACAAGACCTTCAGCGGCAAGGGCCCATGCAACAACGTGAGCACA
GTGCAGTGTACCCACGGCATCAAGCCCGTGGTGTCCACCCAGCTG
CTGCTGAATGGCTCTCTGGCCGAGAAGGAGATCATCATCAGGTCC
GAGAATCTGACAGATAACGTGAAGACCATCATCGTGCACCTGAAC
AAGTCCGTGGAGATCGTGTGCACACGCCCTAACAATAACACCAGG
AAGTCTATGCGCATCGGCCCAGGCCAGACATTCTACGCCACCGGC
GACATCATCGGCGATATCCGGCAGGCCTATTGTAATATCAGCGGC
TCCAAGTGGAACGAGACACTGAAGAGAGTGAAGGAGAAGCTGCAG
GAGAACTACAATAACAATAAGACCATCAAGTTCGCACCAAGCTCC
GGAGGCGATCTGGAGATCACAACCCACAGCTTTAATTGCCGGGGC
GAGTTCTTTTATTGTAACACAACCAGACTGTTCAACAATAACGCC
ACCGAGGACGAGACAATCACCCTGCCTTGCCGGATCAAGCAGATC
ATCAATATGTGGCAGGGAGTGGGAAGAGCAATGTACGCACCACCT
ATCGCCGGCAATATCACCTGTAAGAGCAACATCACCGGACTGCTG
CTGGTGAGAGACGGAGGAGAGGATAACAAGACAGAGGAGATCTTT
CGGCCCGGCGGCGGCAATATGAAGGACAACTGGAGATCCGAGCTG
TACAAGTATAAAGTGATCGAGCTGAAGCCACTGGGAATCGCACCT
ACCGGATGCAAGAGGAGAGTGGTGGAGGGAGGCTCTGGAGGAGGA
GGAAGCGGAGGAGGAGGATCCGGCGGCGCCGTGGGCATCGGAGCC
GTGTTCCTGGGCTTTCTGGGAGCAGCAGGATCTACCATGGGAGCA
GCAAGCCTGACACTGACCGTGCAGGCCAGGCAGCTGCTGTCTAGC
ATCGTGCAGCAGCAGTCCAATCTGCTGAGGGCACCAGAGGCACAG
CAGCACATGCTGCAGCTGACAGTGTGGGGCATCAAGCAGCTGCAG
ACCCGGGTGCTGGCCATCGAGAGATACCTGAAGGATCAGCAGCTG
CTGGGCATCTGGGGCTGCTCTGGCAAGCTGATCTGCTGTACCAAT
GTGCCCTGGAACTCCTCTTGGTCCAACAAGTCTCAGACAGACATC
TGGAATAACATGACCTGGATGGAGTGGGACAGGGAGATCTCTAAT
TACACAGATACCATCTATCGCCTGCTGGAGGACAGCCAGACCCAG
CAGGAGAAGAACGAGAAGGACCTGCTGGCCCTGGATGGAAGCGGA
GATATCATCAAGCTGCTGAACGAGCAAGTGAATAAGGAGATGCAG
AGCTCCAACCTGTACATGAGCATGTCTAGCTGGTGCTATACCCAC
TCCCTGGACGGAGCAGGACTGTTCCTGTTTGATCACGCCGCCGAG
GAGTATGAGCACGCCAAGAAGCTGATCATCTTTCTGAATGAGAAC
AATGTGCCCGTGCAGCTGACCTCCATCTCTGCCCCTGAGCACAAG
TTCGAGGGCCTGACACAGATCTTTCAGAAGGCCTACGAGCACGAG
CAGCACATCAGCGAGTCCATCAACAATATCGTGGACCACGCCATC
AAGTCCAAGGATCACGCCACATTCAACTTTCTGCAGTGGTACGTG
GCCGAGCAGCACGAGGAGGAGGTGCTGTTCAAGGACATCCTGGAT
AAGATCGAGCTGATCGGCAACGAGAATCACGGCCTGTACCTGGCC
GACCAGTATGTGAAGGGCATCGCCAAGTCTCGGAAGAGC,
SEQ ID NO: 2
Protein sequence for
BG505.SOSIP.664scLinkerFerritin_2A_
CZA97.SOSIP.664scLinkerFerritin
MPMGSLQPLATLYLLGMLVASVLAAENLWVTVYYGVPVWKDAETT
LFCASDAKAYETEKHNVWATHACVPTDPNPQEIHLENVTEEFNMW
KNNMVEQMHTDIISLWDQSLKPCVKLTPLCVTLQCTNVTNNITDD
MRGELKNCSFNMTTELRDKKQKVYSLFYRLDVVQINENQGNRSNN
SNKEYRLINCNTSAITQACPKVSFEPIPIHYCAPAGFAILKCKDK
KFNGTGPCPSVSTVQCTHGIKPVVSTQLLLNGSLAEEEVMIRSEN
ITNNAKNILVQFNTPVQINCTRPNNNTRKSIRIGPGQAFYATGDI
IGDIRQAHCTVSKATWNETLGKVVKQLRKHFGNNTIIRFANSSGG
DLEVTTHSFNCGGEFFYCNTSGLFNSTWISNTSVQGSNSTGSNDS
ITLPCRIKQIINMWQRIGQAMYAPPIQGVIRCVSNITGLILTRDG
GSTNSTTETFRPGGGDMRDNWRSELYKYKVVKIEPLGVAPTRCKR
RVVGGGSGGGGSGGGGSGGAVGIGAVFLGFLGAAGSTMGAASMTL
TVQARNLLSGIVQQQSNLLRAPEAQQHLLKLTVWGIKQLQARVLA
VERYLRDQQLLGIWGCSGKLICCTNVPWNSSWSNRNLSEIWDNMT
WLQWDKEISNYTQIIYGLLEESQNQQEKNEQDLLALDGSGDIIKL
LNEQVNKEMQSSNLYMSMSSWCYTHSLDGAGLFLFDHAAEEYEHA
KKLIIFLNENNVPVQLTSISAPEHKFEGLTQIFQKAYEHEQHISE
SINNIVDHAIKSKDHATFNFLQWYVAEQHEEEVLFKDILDKIELI
GNENHGLYLADQYVKGIAKSRKSGSGATNFSLLKQAGDVEENPGP
GSGMPMGSLQPLATLYLLGMLVASVLAVGNMWVTVYYGVPVWTDA
KTTLFCASDTKAYDREVHNVWATHACVPTDPNPQEIVLENVTENF
NMWKNDMVDQMHEDIISLWDQSLKPCVKLTPLCVTLHCTNATFKN
NVTNDMNKEIRNCSFNTTTEIRDKKQQGYALFYRPDIVLLKENRN
NSNNSEYILINCNASTITQACPKVNFDPIPIHYCAPAGYAILKCN
NKTFSGKGPCNNVSTVQCTHGIKPVVSTQLLLNGSLAEKEIIIRS
ENLTDNVKTIIVHLNKSVEIVCTRPNNNTRKSMRIGPGQTFYATG
DIIGDIRQAYCNISGSKWNETLKRVKEKLQENYNNNKTIKFAPSS
GGDLEITTHSFNCRGEFFYCNTTRLFNNNATEDETITLPCRIKQI
INMWQGVGRAMYAPPIAGNITCKSNITGLLLVRDGGEDNKTEEIF
RPGGGNMKDNWRSELYKYKVIELKPLGIAPTGCKRRVVEGGSGGG
GSGGGGSGGAVGIGAVFLGFLGAAGSTMGAASLTLTVQARQLLSS
IVQQQSNLLRAPEAQQHMLQLTVWGIKQLQTRVLAIERYLKDQQL
LGIWGCSGKLICCTNVPWNSSWSNKSQTDIWNNMTWMEWDREISN
YTDTIYRLLEDSQTQQEKNEKDLLALDGSGDIIKLLNEQVNKEMQ
SSNLYMSMSSWCYTHSLDGAGLFLFDHAAEEYEHAKKLIIFLNEN
NVPVQLTSISAPEHKFEGLTQIFQKAYEHEQHISESINNIVDHAI
KSKDHATFNFLQWYVAEQHEEEVLFKDILDKIELIGNENHGLYLA
DQYVKGIAKSRKS
SEQ ID NO: 3
DNA sequence,
BG505.SOSIP.664sc_2A_CZA97.SOSIP.664sc
atgcccatgggcagcctgcagcccctggccaccctgtacctgctg
ggcatgctggtggctagcgtgctggccgccgaaaacctgtgggtc
accgtgtattatggagtgcccgtctggaaagatgctgaaactacc
ctgttctgtgcctctgatgctaaggcctacgagaccgaaaagcac
aatgtctgggctactcatgcatgcgtgcccaccgacccaaacccc
caggagatccacctggaaaatgtgaccgaggaattcaacatgtgg
aaaaacaatatggtggagcagatgcatacagacatcattagcctg
tgggatcagtccctgaagccctgcgtcaaactgactcctctgtgc
gtgaccctgcagtgtaccaatgtcacaaacaatatcaccgacgat
atgaggggcgagctgaagaattgtagcttcaacatgaccacagaa
ctgagagacaagaaacagaaagtgtactccctgttttataggctg
gatgtggtccagatcaatgagaaccaggggaatcggagcaacaat
tccaacaaggaatacagactgatcaattgcaacacttccgccatt
acccaggcttgtcctaaagtgtcttttgagcctatcccaattcat
tattgcgccccagctggcttcgccatcctgaagtgtaaagataag
aagttcaacggaactggcccctgcccttccgtgtctacagtccag
tgtactcacgggattaagcctgtggtctctacacagctgctgctg
aatggaagtctggctgaggaagaagtgatgatccggagcgagaac
attaccaacaatgccaagaatatcctggtccagttcaacacacca
gtgcagattaattgcacaagacccaacaataacactcgaaaatct
atccggattgggccaggacaggccttttacgctacaggggacatc
attggagatatcagacaggctcactgtaCCgtgagtaaggcaacc
tggaacgagacactgggcaaggtggtcaaacagctgaggaaacat
ttcgggaataacaccatcattcgctttgccaatagctccggaggg
gacctggaggtcactacccactccttcaactgcggaggcgaattc
ttttactgtaacacatctggcctgtttaatagtacatggatctct
aacactagtgtgcagggcagtaattcaactgggtcaaacgatagc
atcaccctgccatgccgaattaagcagatcattaatatgtggcag
cggatcggccaggcaatgtatgccccccctatccagggggtcatt
cgctgcgtgagcaatatcaccggactgattctgacacgagacggg
ggcagcaccaactctacaactgaaacattccggcccggcggggga
gacatgagagataactggaggtccgagctgtacaagtataaagtg
gtcaagatcgaacctctgggagtggcaccaaccagatgcaagcga
agagtggtcggaGGCGGCAGCGGCGGCGGCGGCTCCGGCGGCGGC
GGCTCTGGCGGCgcagtcggaattggggccgtgttcctgggattt
ctgggcgccgctgggagtacaatgggagcagcctcaatgactctg
accgtgcaggccaggaatctgctgagcggcatcgtccagcagcag
tccaacctgctgcgcgctcctgaagcacagcagcacctgctgaag
ctgaccgtgtggggcatcaaacagctgcaggctagggtgctggca
gtcgagcggtacctgagagaccagcagctgctgggaatctggggc
tgctctgggaagctgatttgttgcacaaatgtgccttggaactct
agttggtcaaatcgcaacctgagcgagatctgggacaatatgact
tggctgcagtgggataaagaaattagtaactacacccagatcatc
tacggcctgctggaagagtcacagaatcagcaggagaagaacgaa
caggacctgctggcactggatGGCAGCGGCgctactaacttcagc
ctgctgaagcaggctggagacgtggaggagaaccctggacctgga
agcggaAtgcccatgggcagcctgcagcccctggccaccctgtac
ctgctgggcatgctggtggctagcgtgctggccGTGGGCAACATG
TGGGTGACAGTGTACTATGGCGTGCCCGTGTGGACCGATGCCAAG
ACCACACTGTTCTGCGCCTCCGACACAAAGGCCTACGATCGGGAG
GTGCACAACGTGTGGGCAACACACGCATGCGTGCCAACCGACCCA
AATCCCCAGGAGATCGTGCTGGAGAACGTGACCGAGAACTTCAAC
ATGTGGAAGAACGACATGGTGGATCAGATGCACGAGGACATCATC
AGCCTGTGGGATCAGTCCCTGAAGCCATGCGTGAAGCTGACACCC
CTGTGCGTGACCCTGCACTGTACAAACGCCACCTTTAAGAACAAT
GTGACCAATGATATGAACAAGGAGATCAGGAATTGTTCTTTCAAC
ACCACAACCGAGATCCGCGATAAGAAGCAGCAGGGCTACGCCCTG
TTTTATAGGCCTGACATCGTGCTGCTGAAGGAGAATCGCAACAAT
TCTAACAATAGCGAGTATATCCTGATCAATTGCAACGCCAGCACA
ATCACCCAGGCCTGTCCCAAGGTGAACTTCGACCCTATCCCAATC
CACTACTGCGCCCCTGCCGGCTATGCCATCCTGAAGTGTAACAAC
AAGACCTTCAGCGGCAAGGGCCCATGCAACAACGTGAGCACAGTG
CAGTGTACCCACGGCATCAAGCCCGTGGTGTCCACCCAGCTGCTG
CTGAATGGCTCTCTGGCCGAGAAGGAGATCATCATCAGGTCCGAG
AATCTGACAGATAACGTGAAGACCATCATCGTGCACCTGAACAAG
TCCGTGGAGATCGTGTGCACACGCCCTAACAATAACACCAGGAAG
TCTATGCGCATCGGCCCAGGCCAGACATTCTACGCCACCGGCGAC
ATCATCGGCGATATCCGGCAGGCCTATTGTAATATCAGCGGCTCC
AAGTGGAACGAGACACTGAAGAGAGTGAAGGAGAAGCTGCAGGAG
AACTACAATAACAATAAGACCATCAAGTTCGCACCAAGCTCCGGA
GGCGATCTGGAGATCACAACCCACAGCTTTAATTGCCGGGGCGAG
TTCTTTTATTGTAACACAACCAGACTGTTCAACAATAACGCCACC
GAGGACGAGACAATCACCCTGCCTTGCCGGATCAAGCAGATCATC
AATATGTGGCAGGGAGTGGGAAGAGCAATGTACGCACCACCTATC
GCCGGCAATATCACCTGTAAGAGCAACATCACCGGACTGCTGCTG
GTGAGAGACGGAGGAGAGGATAACAAGACAGAGGAGATCTTTCGG
CCCGGCGGCGGCAATATGAAGGACAACTGGAGATCCGAGCTGTAC
AAGTATAAAGTGATCGAGCTGAAGCCACTGGGAATCGCACCTACC
GGATGCAAGAGGAGAGTGGTGGAGGGAGGCTCTGGAGGAGGAGGA
AGCGGAGGAGGAGGATCCGGCGGCGCCGTGGGCATCGGAGCCGTG
TTCCTGGGCTTTCTGGGAGCAGCAGGATCTACCATGGGAGCAGCA
AGCCTGACACTGACCGTGCAGGCCAGGCAGCTGCTGTCTAGCATC
GTGCAGCAGCAGTCCAATCTGCTGAGGGCACCAGAGGCACAGCAG
CACATGCTGCAGCTGACAGTGTGGGGCATCAAGCAGCTGCAGACC
CGGGTGCTGGCCATCGAGAGATACCTGAAGGATCAGCAGCTGCTG
GGCATCTGGGGCTGCTCTGGCAAGCTGATCTGCTGTACCAATGTG
CCCTGGAACTCCTCTTGGTCCAACAAGTCTCAGACAGACATCTGG
AATAACATGACCTGGATGGAGTGGGACAGGGAGATCTCTAATTAC
ACAGATACCATCTATCGCCTGCTGGAGGACAGCCAGACCCAGCAG
GAGAAGAACGAGAAGGACCTGCTGGCCCTGGATtga,
SEQ ID NO: 4
Protein sequence,
BG505.SOSIP.664sc_2A_CZA97.SOSIP.664sc
MPMGSLQPLATLYLLGMLVASVLAAENLWVTVYYGVPVWKDAETT
LFCASDAKAYETEKHNVWATHACVPTDPNPQEIHLENVTEEFNMW
KNNMVEQMHTDIISLWDQSLKPCVKLTPLCVTLQCTNVTNNITDD
MRGELKNCSFNMTTELRDKKQKVYSLFYRLDVVQINENQGNRSNN
SNKEYRLINCNTSAITQACPKVSFEPIPIHYCAPAGFAILKCKDK
KFNGTGPCPSVSTVQCTHGIKPVVSTQLLLNGSLAEEEVMIRSEN
ITNNAKNILVQFNTPVQINCTRPNNNTRKSIRIGPGQAFYATGDI
IGDIRQAHCTVSKATWNETLGKVVKQLRKHFGNNTIIRFANSSGG
DLEVTTHSFNCGGEFFYCNTSGLFNSTWISNTSVQGSNSTGSNDS
ITLPCRIKQIINMWQRIGQAMYAPPIQGVIRCVSNITGLILTRDG
GSTNSTTETFRPGGGDMRDNWRSELYKYKVVKIEPLGVAPTRCKR
RVVGGGSGGGGSGGGGSGGAVGIGAVFLGFLGAAGSTMGAASMTL
TVQARNLLSGIVQQQSNLLRAPEAQQHLLKLTVWGIKQLQARVLA
VERYLRDQQLLGIWGCSGKLICCTNVPWNSSWSNRNLSEIWDNMT
WLQWDKEISNYTQIIYGLLEESQNQQEKNEQDLLALDGSGGSGAT
NFSLLKQAGDVEENPGPGSGMPMGSLQPLATLYLLGMLVASVLAV
GNMWVTVYYGVPVWTDAKTTLFCASDTKAYDREVHNVWATHACVP
TDPNPQEIVLENVTENFNMWKNDMVDQMHEDIISLWDQSLKPCVK
LTPLCVTLHCTNATFKNNVTNDMNKEIRNCSFNTTTEIRDKKQQG
YALFYRPDIVLLKENRNNSNNSEYILINCNASTITQACPKVNFDP
IPIHYCAPAGYAILKCNNKTFSGKGPCNNVSTVQCTHGIKPVVST
QLLLNGSLAEKEIIIRSENLTDNVKTIIVHLNKSVEIVCTRPNNN
TRKSMRIGPGQTFYATGDIIGDIRQAYCNISGSKWNETLKRVKEK
LQENYNNNKTIKFAPSSGGDLEITTHSFNCRGEFFYCNTTRLFNN
NATEDETITLPCRIKQIINMWQGVGRAMYAPPIAGNITCKSNITG
LLLVRDGGEDNKTEEIFRPGGGNMKDNWRSELYKYKVIELKPLGI
APTGCKRRVVEGGSGGGGSGGGGSGGAVGIGAVFLGFLGAAGSTM
GAASLTLTVQARQLLSSIVQQQSNLLRAPEAQQHMLQLTVWGIKQ
LQTRVLAIERYLKDQQLLGIWGCSGKLICCTNVPWNSSWSNKSQT
DIWNNMTWMEWDREISNYTDTIYRLLEDSQTQQEKNEKDLLALD,
SEQ ID NO: 5
(DNA sequence, BG505)
gccgaaaacctgtgggtcaccgtgtattatggagtgcccgtctgg
aaagatgctgaaactaccctgttctgtgcctctgatgctaaggcc
tacgagaccgaaaagcacaatgtctgggctactcatgcatgcgtg
cccaccgacccaaacccccaggagatccacctggaaaatgtgacc
gaggaattcaacatgtggaaaaacaatatggtggagcagatgcat
acagacatcattagcctgtgggatcagtccctgaagccctgcgtc
aaactgactcctctgtgcgtgaccctgcagtgtaccaatgtcaca
aacaatatcaccgacgatatgaggggcgagctgaagaattgtagc
ttcaacatgaccacagaactgagagacaagaaacagaaagtgtac
tccctgttttataggctggatgtggtccagatcaatgagaaccag
gggaatcggagcaacaattccaacaaggaatacagactgatcaat
tgcaacacttccgccattacccaggcttgtcctaaagtgtctttt
gagcctatcccaattcattattgcgccccagctggcttcgccatc
ctgaagtgtaaagataagaagttcaacggaactggcccctgccct
tccgtgtctacagtccagtgtactcacgggattaagcctgtggtc
tctacacagctgctgctgaatggaagtctggctgaggaagaagtg
atgatccggagcgagaacattaccaacaatgccaagaatatcctg
gtccagttcaacacaccagtgcagattaattgcacaagacccaac
aataacactcgaaaatctatccggattgggccaggacaggccttt
tacgctacaggggacatcattggagatatcagacaggctcactgt
aCCgtgagtaaggcaacctggaacgagacactgggcaaggtggtc
aaacagctgaggaaacatttcgggaataacaccatcattcgcttt
gccaatagctccggaggggacctggaggtcactacccactccttc
aactgcggaggcgaattcttttactgtaacacatctggcctgttt
aatagtacatggatctctaacactagtgtgcagggcagtaattca
actgggtcaaacgatagcatcaccctgccatgccgaattaagcag
atcattaatatgtggcagcggatcggccaggcaatgtatgccccc
cctatccagggggtcattcgctgcgtgagcaatatcaccggactg
attctgacacgagacgggggcagcaccaactctacaactgaaaca
ttccggcccggcgggggagacatgagagataactggaggtccgag
ctgtacaagtataaagtggtcaagatcgaacctctgggagtggca
ccaaccagatgcaagcgaagagtggtcggaGGCGGCAGCGGCGGC
GGCGGCTCCGGCGGCGGCGGCTCTGGCGGCgcagtcggaattggg
gccgtgttcctgggatttctgggcgccgctgggagtacaatggga
gcagcctcaatgactctgaccgtgcaggccaggaatctgctgagc
ggcatcgtccagcagcagtccaacctgctgcgcgctcctgaagca
cagcagcacctgctgaagctgaccgtgtggggcatcaaacagctg
caggctagggtgctggcagtcgagcggtacctgagagaccagcag
ctgctgggaatctggggctgctctgggaagctgatttgttgcaca
aatgtgccttggaactctagttggtcaaatcgcaacctgagcgag
atctgggacaatatgacttggctgcagtgggataaagaaattagt
aactacacccagatcatctacggcctgctggaagagtcacagaat
cagcaggagaagaacgaacaggacctgctggcactggat
SEQ ID NO: 6
(Protein sequence, BG505)
AENLWVTVYYGVPVWKDAETTLFCASDAKAYETEKHNVWATHACV
PTDPNPQEIHLENVTEEFNMWKNNMVEQMHTDIISLWDQSLKPCV
KLTPLCVTLQCTNVTNNITDDMRGELKNCSFNMTTELRDKKQKVY
SLFYRLDVVQINENQGNRSNNSNKEYRLINCNTSAITQACPKVSF
EPIPIHYCAPAGFAILKCKDKKFNGTGPCPSVSTVQCTHGIKPVV
STQLLLNGSLAEEEVMIRSENITNNAKNILVQFNTPVQINCTRPN
NNTRKSIRIGPGQAFYATGDIIGDIRQAHCTVSKATWNETLGKVV
KQLRKHFGNNTIIRFANSSGGDLEVTTHSFNCGGEFFYCNTSGLF
NSTWISNTSVQGSNSTGSNDSITLPCRIKQIINMWQRIGQAMYAP
PIQGVIRCVSNITGLILTRDGGSTNSTTETFRPGGGDMRDNWRSE
LYKYKVVKIEPLGVAPTRCKRRVVGGGSGGGGSGGGGSGGAVGIG
AVFLGFLGAAGSTMGAASMTLTVQARNLLSGIVQQQSNLLRAPEA
QQHLLKLTVWGIKQLQARVLAVERYLRDQQLLGIWGCSGKLICCT
NVPWNSSWSNRNLSEIWDNMTWLQWDKEISNYTQIIYGLLEESQN
QQEKNEQDLLALD
SEQ ID NO: 7
(DNA sequence, CZA97)
GTGGGCAACATGTGGGTGACAGTGTACTATGGCGTGCCCGTGTGG
ACCGATGCCAAGACCACACTGTTCTGCGCCTCCGACACAAAGGCC
TACGATCGGGAGGTGCACAACGTGTGGGCAACACACGCATGCGTG
CCAACCGACCCAAATCCCCAGGAGATCGTGCTGGAGAACGTGACC
GAGAACTTCAACATGTGGAAGAACGACATGGTGGATCAGATGCAC
GAGGACATCATCAGCCTGTGGGATCAGTCCCTGAAGCCATGCGTG
AAGCTGACACCCCTGTGCGTGACCCTGCACTGTACAAACGCCACC
TTTAAGAACAATGTGACCAATGATATGAACAAGGAGATCAGGAAT
TGTTCTTTCAACACCACAACCGAGATCCGCGATAAGAAGCAGCAG
GGCTACGCCCTGTTTTATAGGCCTGACATCGTGCTGCTGAAGGAG
AATCGCAACAATTCTAACAATAGCGAGTATATCCTGATCAATTGC
AACGCCAGCACAATCACCCAGGCCTGTCCCAAGGTGAACTTCGAC
CCTATCCCAATCCACTACTGCGCCCCTGCCGGCTATGCCATCCTG
AAGTGTAACAACAAGACCTTCAGCGGCAAGGGCCCATGCAACAAC
GTGAGCACAGTGCAGTGTACCCACGGCATCAAGCCCGTGGTGTCC
ACCCAGCTGCTGCTGAATGGCTCTCTGGCCGAGAAGGAGATCATC
ATCAGGTCCGAGAATCTGACAGATAACGTGAAGACCATCATCGTG
CACCTGAACAAGTCCGTGGAGATCGTGTGCACACGCCCTAACAAT
AACACCAGGAAGTCTATGCGCATCGGCCCAGGCCAGACATTCTAC
GCCACCGGCGACATCATCGGCGATATCCGGCAGGCCTATTGTAAT
ATCAGCGGCTCCAAGTGGAACGAGACACTGAAGAGAGTGAAGGAG
AAGCTGCAGGAGAACTACAATAACAATAAGACCATCAAGTTCGCA
CCAAGCTCCGGAGGCGATCTGGAGATCACAACCCACAGCTTTAAT
TGCCGGGGCGAGTTCTTTTATTGTAACACAACCAGACTGTTCAAC
AATAACGCCACCGAGGACGAGACAATCACCCTGCCTTGCCGGATC
AAGCAGATCATCAATATGTGGCAGGGAGTGGGAAGAGCAATGTAC
GCACCACCTATCGCCGGCAATATCACCTGTAAGAGCAACATCACC
GGACTGCTGCTGGTGAGAGACGGAGGAGAGGATAACAAGACAGAG
GAGATCTTTCGGCCCGGCGGCGGCAATATGAAGGACAACTGGAGA
TCCGAGCTGTACAAGTATAAAGTGATCGAGCTGAAGCCACTGGGA
ATCGCACCTACCGGATGCAAGAGGAGAGTGGTGGAGGGAGGCTCT
GGAGGAGGAGGAAGCGGAGGAGGAGGATCCGGCGGCGCCGTGGGC
ATCGGAGCCGTGTTCCTGGGCTTTCTGGGAGCAGCAGGATCTACC
ATGGGAGCAGCAAGCCTGACACTGACCGTGCAGGCCAGGCAGCTG
CTGTCTAGCATCGTGCAGCAGCAGTCCAATCTGCTGAGGGCACCA
GAGGCACAGCAGCACATGCTGCAGCTGACAGTGTGGGGCATCAAG
CAGCTGCAGACCCGGGTGCTGGCCATCGAGAGATACCTGAAGGAT
CAGCAGCTGCTGGGCATCTGGGGCTGCTCTGGCAAGCTGATCTGC
TGTACCAATGTGCCCTGGAACTCCTCTTGGTCCAACAAGTCTCAG
ACAGACATCTGGAATAACATGACCTGGATGGAGTGGGACAGGGAG
ATCTCTAATTACACAGATACCATCTATCGCCTGCTGGAGGACAGC
CAGACCCAGCAGGAGAAGAACGAGAAGGACCTGCTGGCCCTGGAT
SEQ ID NO: 8
(Protein sequence, CZA97)
AVGNMWVTVYYGVPVWTDAKTTLFCASDTKAYDREVHNVWATHAC
VPTDPNPQEIVLENVTENFNMWKNDMVDQMHEDIISLWDQSLKPC
VKLTPLCVTLHCTNATFKNNVTNDMNKEIRNCSFNTTTEIRDKKQ
QGYALFYRPDIVLLKENRNNSNNSEYILINCNASTITQACPKVNF
DPIPIHYCAPAGYAILKCNNKTFSGKGPCNNVSTVQCTHGIKPVV
STQLLLNGSLAEKEIIIRSENLTDNVKTIIVHLNKSVEIVCTRPN
NNTRKSMRIGPGQTFYATGDIIGDIRQAYCNISGSKWNETLKRVK
EKLQENYNNNKTIKFAPSSGGDLEITTHSFNCRGEFFYCNTTRLF
NNNATEDETITLPCRIKQIINMWQGVGRAMYAPPIAGNITCKSNI
TGLLLVRDGGEDNKTEEIFRPGGGNMKDNWRSELYKYKVIELKPL
GIAPTGCKRRVVEGGSGGGGSGGGGSGGAVGIGAVFLGFLGAAGS
TMGAASLTLTVQARQLLSSIVQQQSNLLRAPEAQQHMLQLTVWGI
KQLQTRVLAIERYLKDQQLLGIWGCSGKLICCTNVPWNSSWSNKS
QTDIWNNMTWMEWDREISNYTDTIYRLLEDSQTQQEKNEKDLLAL
D
SEQ ID NO: 9
(DNA sequence, ferritin)
GATATCATCAAGCTGCTGAACGAGCAAGTGAATAAGGAGATGCAG
AGCTCCAACCTGTACATGAGCATGTCTAGCTGGTGCTATACCCAC
TCCCTGGACGGAGCAGGACTGTTCCTGTTTGATCACGCCGCCGAG
GAGTATGAGCACGCCAAGAAGCTGATCATCTTTCTGAATGAGAAC
AATGTGCCCGTGCAGCTGACCTCCATCTCTGCCCCTGAGCACAAG
TTCGAGGGCCTGACACAGATCTTTCAGAAGGCCTACGAGCACGAG
CAGCACATCAGCGAGTCCATCAACAATATCGTGGACCACGCCATC
AAGTCCAAGGATCACGCCACATTCAACTTTCTGCAGTGGTACGTG
GCCGAGCAGCACGAGGAGGAGGTGCTGTTCAAGGACATCCTGGAT
AAGATCGAGCTGATCGGCAACGAGAATCACGGCCTGTACCTGGCC
GACCAGTATGTGAAGGGCATCGCCAAGTCTCGGAAGAGC
SEQ ID NO: 10
(Protein sequence, ferritin)
DIIKLLNEQVNKEMQSSNLYMSMSSWCYTHSLDGAGLFLFDHAAE
EYEHAKKLIIFLNENNVPVQLTSISAPEHKFEGLTQIFQKAYEHE
QHISESINNIVDHAIKSKDHATFNFLQWYVAEQHEEEVLFKDILD
KIELIGNENHGLYLADQYVKGIAKSRKS
SEQ ID NO: 11
(DNA sequence, 2A_1)
Ggaagcggagctactaacttcagcctgctgaagcaggctggagac
gtggaggagaaccctggacctggaagcgga
SEQ ID NO: 12
(DNA sequence, 2A_2)
GGCAGCGGCgctactaacttcagcctgctgaagcaggctggagac
gtggaggagaaccctggacctggaagcgga
SEQ ID NO: 13
(Protein sequence, 2A_1)
GSGATNFSLLKQAGDVEENPGPGSG
SEQ ID NO: 14
(Protein sequence, 2A_2)
GSGGSGATNFSLLKQAGDVEENPGPGSG
SEQ ID NO: 15
(DNA sequence, Signal Peptide)
ATGCCCATGGGCAGCCTGCAGCCCCTGGCCACCCTGTACCTGCTG
GGCATGCTGGTGGCTAGCGTGCTGGCC
SEQ ID NO: 16
(Protein sequence, Signal Peptide)
MPMGSLQPLATLYLLGMLVASVLA
SEQ ID NO: 17
(DNA sequence, Fusion Peptide_1)
GCGGTTGGTATCGGTGCGGTTTTC
SEQ ID NO: 18
(DNA sequence, Fusion Peptide_2)
CGCGGTTGGTCTCGGTGCGGTTTTC
SEQ ID NO: 19
(DNA sequence, Fusion Peptide_3)
GCGGTTGGTCTCGGTGCGATGATC
SEQ ID NO: 20
(Protein sequence, Fusion Peptide_l)
AVGIGAVF
SEQ ID NO: 21
(Protein sequence, Fusion Peptide_2)
AVGLGAVF
SEQ ID NO: 22
(Protein sequence, Fusion Peptide_3)
AVGLGAMI
SEQ ID NO: 23
(Protein sequence, Fusion Peptide_4)
AVGIGAMI
SEQ ID NO: 24
(Protein sequence, Fusion Peptide_5)
AVGLGAVL
SEQ ID NO: 25
(DNA sequence, linker)
GGAAGCGGA
SEQ ID NO: 26
(DNA sequence, linker)
AGCGGA
SEQ ID NO: 27
(DNA sequence, linker)
AGCGGA
SEQ ID NO: 28
(DNA sequence, linker)
GGCAGCGGC
SEQ ID NO: 29
(Protein sequence, linker)
GSG
SEQ ID NO: 30
(Protein sequence, linker)
GSGGSG
SEQ ID NO: 31
Protein sequence, Strain: 286.36
MKVMGIPKNWPRWWMWGILGLWMLLICNGEDLWVTVYYGVPVWKE
ANPTLFCASDAKAYKTEMHNVWATHACVPTDPNPQEMVLENVTED
FNMWKNGMVEQMHQDIISLWDQSLKPCVKLTPLCVTLNCTEVTRS
SNGTINNNSTEMKNCSFNVTTDLRDKKKKEHALFYRLDIVPLDET
NGTSSEYRLINCNTSTITQACPKVSFDPIPIHYCAPAGYAILKCK
DKKFNGTGPCKNVSTVQCTHGIKPVVSTQLLLNGSIAEGEIIIRS
ENLTNNAKIIIVQLNVTVEINCTRPNNNTRRSIRIGPGQTFYATG
EIIGDIRQAHCNISREKWNRTLQKVEKKLEELFPNKTIHFTSSSG
GDLEITTHSFNCMGEFFYCNTSALFNNNNDSTNSNITLPCRIRQF
INMWQEVGRAMYAPPIQGVITCKSNVTGLLLTRDGGIINDTEIFR
PGGGDMRDNWRSELYKYKVVEIKPLGIAPTTAKRRVVEREKRAVG
IGAVFLGFLGAAGSTMGAASITLTAQARQLLSGIVQQQSNLLRAI
EAQQHMLQLTVWGIKQLQTRVLAIERYLKDQQLLGIWGCSGKLIC
TTAVPWNGSWSNKSQDEIWHNMTWMQWDKEINNYTNIIYGLLEVS
QNQQEKNEQDLLALDKWQNLWSWFNITNWLWYIKIFIMIVGGLIG
LRIIFTVLSIVNRVRQGYSPLSFQTLIPNPRGPDRPRGIEEEGGE
QDRSRSIRLVSGFLALAWDDLRSLCLFSYHRLRDLILIAARVVEL
LGQRGWEALKYLGSLVQYWGLELKKSAISLFDTIAIAVAEGTDRI
IEVLQGIGRAICNIPRRIRQGFEAALQ,
SEQ ID NO: 32
Protein sequence, Strain: 286.36
(DS.SOSIP.664.sc)
MPMGSLQPLATLYLLGMLVASVLAGEDLWVTVYYGVPVWKEANPT
LFCASDAKAYKTEMHNVWATHACVPTDPNPQEMVLENVTEDFNMW
KNGMVEQMHQDIISLWDQSLKPCVKLTPLCVTLNCTEVTRSSNGT
INNNSTEMKNCSFNVTTDLRDKKKKEHALFYRLDIVPLDETNGTS
SEYRLINCNTSTCTQACPKVSFDPIPIHYCAPAGYAILKCKDKKF
NGTGPCKNVSTVQCTHGIKPVVSTQLLLNGSIAEGEIIIRSENLT
NNAKIIIVQLNVTVEINCTRPNNNTRRSIRIGPGQTFYATGEIIG
DIRQAHCNISREKWNRTLQKVEKKLEELFPNKTIHFTSSSGGDLE
ITTHSFNCMGEFFYCNTSALFNNNNDSTNSNITLPCRIRQFINMW
QEVGRCMYAPPIQGVITCKSNVTGLLLTRDGGIINDTEIFRPGGG
DMRDNWRSELYKYKVVEIKPLGIAPTTCKRRVVEGGSGGGGSGGG
GSGGAVGIGAVFLGFLGAAGSTMGAASITLTAQARQLLSGIVQQQ
SNLLRAPEAQQHMLQLTVWGIKQLQTRVLAIERYLKDQQLLGIWG
CSGKLICCTAVPWNGSWSNKSQDEIWHNMTWMQWDKEINNYTNII
YGLLEVSQNQQEKNEQDLLALD,
SEQ ID NO: 33
Protein sequence,
Strain: 286.36 (DS.SOSIP.sc + MPER)
MPMGSLQPLATLYLLGMLVASVLAGEDLWVTVYYGVPVWKEANPT
LFCASDAKAYKTEMHNVWATHACVPTDPNPQEMVLENVTEDFNMW
KNGMVEQMHQDIISLWDQSLKPCVKLTPLCVTLNCTEVTRSSNGT
INNNSTEMKNCSFNVTTDLRDKKKKEHALFYRLDIVPLDETNGTS
SEYRLINCNTSTCTQACPKVSFDPIPIHYCAPAGYAILKCKDKKF
NGTGPCKNVSTVQCTHGIKPVVSTQLLLNGSIAEGEIIIRSENLT
NNAKIIIVQLNVTVEINCTRPNNNTRRSIRIGPGQTFYATGEIIG
DIRQAHCNISREKWNRTLQKVEKKLEELFPNKTIHFTSSSGGDLE
ITTHSFNCMGEFFYCNTSALFNNNNDSTNSNITLPCRIRQFINMW
QEVGRCMYAPPIQGVITCKSNVTGLLLTRDGGIINDTEIFRPGGG
DMRDNWRSELYKYKVVEIKPLGIAPTTCKRRVVEGGSGGGGSGGG
GSGGAVGIGAVFLGFLGAAGSTMGAASITLTAQARQLLSGIVQQQ
SNLLRAPEAQQHMLQLTVWGIKQLQTRVLAIERYLKDQQLLGIWG
CSGKLICCTAVPWNGSWSNKSQDEIWHNMTWMQWDKEINNYTNII
YGLLEVSQNQQEKNEQDLLALDKWQNLWSWFNITNWLWYIKIFIM
IVGGLIGLRIIFTVLSIVNRVRQGYSPLSFQTLIPNPRGPDRPRG
IEEEGGEQDRSRSIRLVSGFLALAWDDLRSLCLFSYHRLRDLILI
AARVVELLGQRGWEALKYLGSLVQYWGLELKKSAISLFDTIAIAV
AEGTDRIIEVLQGIGRAICNIPRRIRQGFEAALQ,
SEQ ID NO: 34
Protein sequence, Strain: 286.36
(DS.SOSIP.664.sc) + Insect Ferritin Heavy
Chain
MPMGSLQPLATLYLLGMLVASVLAGEDLWVTVYYGVPVWKEANPT
LFCASDAKAYKTEMHNVWATHACVPTDPNPQEMVLENVTEDFNMW
KNGMVEQMHQDIISLWDQSLKPCVKLTPLCVTLNCTEVTRSSNGT
INNNSTEMKNCSFNVTTDLRDKKKKEHALFYRLDIVPLDETNGTS
SEYRLINCNTSTCTQACPKVSFDPIPIHYCAPAGYAILKCKDKKF
NGTGPCKNVSTVQCTHGIKPVVSTQLLLNGSIAEGEIIIRSENLT
NNAKIIIVQLNVTVEINCTRPNNNTRRSIRIGPGQTFYATGEIIG
DIRQAHCNISREKWNRTLQKVEKKLEELFPNKTIHFTSSSGGDLE
ITTHSFNCMGEFFYCNTSALFNNNNDSTNSNITLPCRIRQFINMW
QEVGRCMYAPPIQGVITCKSNVTGLLLTRDGGIINDTEIFRPGGG
DMRDNWRSELYKYKVVEIKPLGIAPTTCKRRVVEGGSGGGGSGGG
GSGGAVGIGAVFLGFLGAAGSTMGAASITLTAQARQLLSGIVQQQ
SNLLRAPEAQQHMLQLTVWGIKQLQTRVLAIERYLKDQQLLGIWG
CSGKLICCTAVPWNGSWSNKSQDEIWHNMTWMQWDKEINNYTNII
YGLLEVSQNQQEKNEQDLLALDGGSGGRSCRNSMRQQIQMEVGAS
LQYLAMGAHFSKDVVNRPGFAQLFFDAASEEREHAMKLIEYLLMR
GELTNDVSSLLQVRPPTRSSWKGGVEALEHALSMESDVTKSIRNV
IKACEDDSEFNDYHLVDYLTGDFLEEQYKGQRDLAGKASTLKKLM
DRHEALGEFIFDKKLLGIDV,
SEQ ID NO: 35
Protein sequence, Strain: 5768.04
MRVKGIKKNYQHWWRWGMMIFGLLMICSAADKLWVTVYYGVPVWK
ETTTTLFCASDARAYDTEVHNVWATHACVPTDPNPQEVVLGNVTE
NFNMWKNNMVEQMHEDIISLWDQSLKPCVRLTPLCVTLNCIDYYG
NTTNSNNSSETMMEKGEIKNCSFNITTRLKDKMQKEYALFYKYDI
VPIDNRVGNDTSNATSYRLTSCNTSVITQACPKVSFEPIPIHYCA
PAGFAILKCNDKKFNGTGPCKNVSTVQCTHGIKPVVSTQLLLNGS
LAEEEVMIRSENFTDNAKTIIVQLNETVEINCTRPNNNTRKSIHM
GPGKVFYTTGEIIGDIRQAHCNINRAKWNNTLIKIVEKLRVKFNK
TISFKQSSGGDPEIEMHSFNCGGEFFYCNTTQLFNSTWFNNATLN
VNSNVTEGSENITLPCRIRQIVNMWQEVGKAMYAPPIQGQIRCSS
NITGLLLTRDGGGSNSSNTSEEVFRPGGGNMRDNWRSELYKYKVV
KIEPLGIAPTKAKRRVVQREKRTVGIGALFLGFLGAAGSTMGAAS
MTLTVQARQLLSGIVQQQNNLLRAIQAQQHLLQLTVWGIKQLQAR
VLAVERYLKDQQLLGIWGCSGKLICTTAVPWNASWSNKSLNEIWD
NMTWMEWEKEIDNYTSLIYTLIEESQNQQEKNEQELLELDKWASL
WNWFSITNWLWYIKIFIMIVGGSIGLRIVFAVLSIVNRVRQGYSP
LSFQTRLPTPRGPDRPEGIEEEGGERDRDRSGQLVNGFLAIIWVD
LRSLCLFSYHRLRDLLLIVARVVELLGRRGWEALNYWWNLLQYWS
QELKKSAISLLNATAIAVAEGTDRVIEVVQRTCRAIIHIPRRIRQ
GLERLLL,
SEQ ID NO: 36
Protein sequence, Strain: 5768.04
(DS.SOSIP.664.sc)
MPMGSLQPLATLYLLGMLVASVLAADKLWVTVYYGVPVWKETTTT
LFCASDARAYDTEVHNVWATHACVPTDPNPQEVVLGNVTENFNMW
KNNMVEQMHEDIISLWDQSLKPCVRLTPLCVTLNCIDYYGNTTNS
NNSSETMMEKGEIKNCSFNITTRLKDKMQKEYALFYKYDIVPIDN
RVGNDTSNATSYRLTSCNTSVCTQACPKVSFEPIPIHYCAPAGFA
ILKCNDKKFNGTGPCKNVSTVQCTHGIKPVVSTQLLLNGSLAEEE
VMIRSENFTDNAKTIIVQLNETVEINCTRPNNNTRKSIHMGPGKV
FYTTGEIIGDIRQAHCNINRAKWNNTLIKIVEKLRVKFNKTISFK
QSSGGDPEIEMHSFNCGGEFFYCNTTQLFNSTWFNNATLNVNSNV
TEGSENITLPCRIRQIVNMWQEVGKCMYAPPIQGQIRCSSNITGL
LLTRDGGGSNSSNTSEEVFRPGGGNMRDNWRSELYKYKVVKIEPL
GIAPTKCKRRVVQGGSGGGGSGGGGSGGTVGIGALFLGFLGAAGS
TMGAASMTLTVQARQLLSGIVQQQNNLLRAPQAQQHLLQLTVWGI
KQLQARVLAVERYLKDQQLLGIWGCSGKLICCTAVPWNASWSNKS
LNEIWDNMTWMEWEKEIDNYTSLIYTLIEESQNQQEKNEQELLEL
D,
SEQ ID NO: 37
Protein sequence, Strain: 5768.04
(DS.SOSIP.sc + MPER)
MPMGSLQPLATLYLLGMLVASVLAADKLWVTVYYGVPVWKETTTT
LFCASDARAYDTEVHNVWATHACVPTDPNPQEVVLGNVTENFNMW
KNNMVEQMHEDIISLWDQSLKPCVRLTPLCVTLNCIDYYGNTTNS
NNSSETMMEKGEIKNCSFNITTRLKDKMQKEYALFYKYDIVPIDN
RVGNDTSNATSYRLTSCNTSVCTQACPKVSFEPIPIHYCAPAGFA
ILKCNDKKFNGTGPCKNVSTVQCTHGIKPVVSTQLLLNGSLAEEE
VMIRSENFTDNAKTIIVQLNETVEINCTRPNNNTRKSIHMGPGKV
FYTTGEIIGDIRQAHCNINRAKWNNTLIKIVEKLRVKFNKTISFK
QSSGGDPEIEMHSFNCGGEFFYCNTTQLFNSTWFNNATLNVNSNV
TEGSENITLPCRIRQIVNMWQEVGKCMYAPPIQGQIRCSSNITGL
LLTRDGGGSNSSNTSEEVFRPGGGNMRDNWRSELYKYKVVKIEPL
GIAPTKCKRRVVQGGSGGGGSGGGGSGGTVGIGALFLGFLGAAGS
TMGAASMTLTVQARQLLSGIVQQQNNLLRAPQAQQHLLQLTVWGI
KQLQARVLAVERYLKDQQLLGIWGCSGKLICCTAVPWNASWSNKS
LNEIWDNMTWMEWEKEIDNYTSLIYTLIEESQNQQEKNEQELLEL
DKWASLWNWFSITNWLWYIKIFIMIVGGSIGLRIVFAVLSIVNRV
RQGYSPLSFQTRLPTPRGPDRPEGIEEEGGERDRDRSGQLVNGFL
AIIWVDLRSLCLFSYHRLRDLLLIVARVVELLGRRGWEALNYWWN
LLQYWSQELKKSAISLLNATAIAVAEGTDRVIEVVQRTCRAIIHI
PRRIRQGLERLLL,
SEQ ID NO: 38
Protein sequence, Strain: 5768.04
(DS.SOSIP.664.sc) + Insect Ferritin Heavy
Chain
MPMGSLQPLATLYLLGMLVASVLAADKLWVTVYYGVPVWKETTTT
LFCASDARAYDTEVHNVWATHACVPTDPNPQEVVLGNVTENFNMW
KNNMVEQMHEDIISLWDQSLKPCVRLTPLCVTLNCIDYYGNTTNS
NNSSETMMEKGEIKNCSFNITTRLKDKMQKEYALFYKYDIVPIDN
RVGNDTSNATSYRLTSCNTSVCTQACPKVSFEPIPIHYCAPAGFA
ILKCNDKKFNGTGPCKNVSTVQCTHGIKPVVSTQLLLNGSLAEEE
VMIRSENFTDNAKTIIVQLNETVEINCTRPNNNTRKSIHMGPGKV
FYTTGEIIGDIRQAHCNINRAKWNNTLIKIVEKLRVKFNKTISFK
QSSGGDPEIEMHSFNCGGEFFYCNTTQLFNSTWFNNATLNVNSNV
TEGSENITLPCRIRQIVNMWQEVGKCMYAPPIQGQIRCSSNITGL
LLTRDGGGSNSSNTSEEVFRPGGGNMRDNWRSELYKYKVVKIEPL
GIAPTKCKRRVVQGGSGGGGSGGGGSGGTVGIGALFLGFLGAAGS
TMGAASMTLTVQARQLLSGIVQQQNNLLRAPQAQQHLLQLTVWGI
KQLQARVLAVERYLKDQQLLGIWGCSGKLICCTAVPWNASWSNKS
LNEIWDNMTWMEWEKEIDNYTSLIYTLIEESQNQQEKNEQELLEL
DGGSGGRSCRNSMRQQIQMEVGASLQYLAMGAHFSKDVVNRPGFA
QLFFDAASEEREHAMKLIEYLLMRGELTNDVSSLLQVRPPTRSSW
KGGVEALEHALSMESDVTKSIRNVIKACEDDSEFNDYHLVDYLTG
DFLEEQYKGQRDLAGKASTLKKLMDRHEALGEFIFDKKLLGIDV,
SEQ ID NO: 39
Protein sequence, Strain: DU172.17
MRVMGILRSYQQWWIWGILGFWMLMICNVWGNLWVTVYYGVPVWK
EAKTTLFCASDAKAHKEEVHNIWATHACVPTDPNPQEIVLKNVTE
NFNMWKNDMVDQMHEDIISLWDQSLKPCVKLTPLCVTLNCSDVKI
KGTNATYNNATYNNNNTISDMKNCSFNTTTEITDKKKKEYALFYK
LDVVALDGKETNSTNSSEYRLINCNTSAVTQACPKVSFDPIPIHY
CAPAGYAILKCNNKTFNGTGPCNNVSTVQCTHGIKPVVSTQLLLN
GSLAEEEVVIRFENLTNNAKIIIVHLNESVEINCTRPSNNTRKSV
RIGPGQTFFATGDIIGDIRQAHCNISRKKWNTTLQRVKEKLKEKF
PNKTIQFAPSSGGDLEITTHSFNCRGEFFYCYTSDLFNSTYMSNN
TGGANITLQCRIKQIIRMWQGVGQAMYAPPIAGNITCKSNITGLL
LTRDGGKEKNDTETFRPGGGDMRDNWRSELYKYKVVEIKPLGIAP
DKAKRRVVEREKRAVGIGAVFLGFLGAAGSTMGAASMTLTVQARQ
LLSGIVQQQSNLLRAIEAQQHMLQLTVWGIKQLQTRVLAIERYLK
DQQLLGIWGCSGKLICTTAVPWNASWSNKSYEEIWGNMTWMQWDR
EINNYTNTIYSLLEESQNQQEKNEKDLLALDSWESLWSWFNITNW
LWYIRIFIIIVGGLIGLRIIFAVLSIVNRVRQGYSPLSFQTLTPS
PREPDRLGRIEEEGGEQDRARSVRLVNGFLALAWEDLRSLCLFSY
HRLRDLILIAARAAALLGRSSLWGLQKGWEALKYLGSLVQYWGLE
LKKSAISLFDAIAITVAEGTDRIINIVQRISRAFYNIPRRIRQGF
EATLQ,
SEQ ID NO: 40
Protein sequence, Strain: DU172.17
(DS.SOSIP.664.sc)
MKAKLLVLLCTFTATYAGNLWVTVYYGVPVWKEAKTTLFCASDAK
AHKEEVHNIWATHACVPTDPNPQEIVLKNVTENFNMWKNDMVDQM
HEDIISLWDQSLKPCVKLTPLCVTLNCSDVKIKGTNATYNNATYN
NNNTISDMKNCSFNTTTEITDKKKKEYALFYKLDVVALDGKETNS
TNSSEYRLINCNTSACTQACPKVSFDPIPIHYCAPAGYAILKCNN
KTFNGTGPCNNVSTVQCTHGIKPVVSTQLLLNGSLAEEEVVIRFE
NLTNNAKIIIVHLNESVEINCTRPSNNTRKSVRIGPGQTFFATGD
IIGDIRQAHCNISRKKWNTTLQRVKEKLKEKFPNKTIQFAPSSGG
DLEITTHSFNCRGEFFYCYTSDLFNSTYMSNNTGGANITLQCRIK
QIIRMWQGVGQCMYAPPIAGNITCKSNITGLLLTRDGGKEKNDTE
TFRPGGGDMRDNWRSELYKYKVVEIKPLGIAPDKCKRRVVEGGSG
GGGSGGGGSGGAVGIGAVFLGFLGAAGSTMGAASMTLTVQARQLL
SGIVQQQSNLLRAPEAQQHMLQLTVWGIKQLQTRVLAIERYLKDQ
QLLGIWGCSGKLICCTAVPWNASWSNKSYEEIWGNMTWMQWDREI
NNYTNTIYSLLEESQNQQEKNEKDLLALD,
SEQ ID NO: 41
Protein sequence, Strain:
DU172.17 (DS.SOSIP.sc + MPER)
MKAKLLVLLCTFTATYAGNLWVTVYYGVPVWKEAKTTLFCASDAK
AHKEEVHNIWATHACVPTDPNPQEIVLKNVTENFNMWKNDMVDQM
HEDIISLWDQSLKPCVKLTPLCVTLNCSDVKIKGTNATYNNATYN
NNNTISDMKNCSFNTTTEITDKKKKEYALFYKLDVVALDGKETNS
TNSSEYRLINCNTSACTQACPKVSFDPIPIHYCAPAGYAILKCNN
KTFNGTGPCNNVSTVQCTHGIKPVVSTQLLLNGSLAEEEVVIRFE
NLTNNAKIIIVHLNESVEINCTRPSNNTRKSVRIGPGQTFFATGD
IIGDIRQAHCNISRKKWNTTLQRVKEKLKEKFPNKTIQFAPSSGG
DLEITTHSFNCRGEFFYCYTSDLFNSTYMSNNTGGANITLQCRIK
QIIRMWQGVGQCMYAPPIAGNITCKSNITGLLLTRDGGKEKNDTE
TFRPGGGDMRDNWRSELYKYKVVEIKPLGIAPDKCKRRVVEGGSG
GGGSGGGGSGGAVGIGAVFLGFLGAAGSTMGAASMTLTVQARQLL
SGIVQQQSNLLRAPEAQQHMLQLTVWGIMQWDREINNYTNTIYSL
LEESQNQQEKNEKDLLALDSWESLWSWFNITNWLWYIRIFIIIVG
GLIGLRIIFAVLSIVNRVRQGYSPLSFQTLTPSPREPDRLGRIEE
EGGEQDRARSVRLVNGFLALAWEDLRSLCLFSYHRLRDLILIAAR
AAALLGRSSLWGLQKGWEALKYLGSLVQYWGLELKKSAISLFDAI
AITVAEGTDRIINIVQRISRAFYNIPRRIRQGFEATLQ,
SEQ ID NO: 42
Protein sequence, Strain: DU172.17
(DS.SOSIP.664.sc) + Insect Ferritin
Light Chain
MKAKLLVLLCTFTATYAGNLWVTVYYGVPVWKEAKTTLFCASDAK
AHKEEVHNIWATHACVPTDPNPQEIVLKNVTENFNMWKNDMVDQM
HEDIISLWDQSLKPCVKLTPLCVTLNCSDVKIKGTNATYNNATYN
NNNTISDMKNCSFNTTTEITDKKKKEYALFYKLDVVALDGKETNS
TNSSEYRLINCNTSACTQACPKVSFDPIPIHYCAPAGYAILKCNN
KTFNGTGPCNNVSTVQCTHGIKPVVSTQLLLNGSLAEEEVVIRFE
NLTNNAKIIIVHLNESVEINCTRPSNNTRKSVRIGPGQTFFATGD
IIGDIRQAHCNISRKKWNTTLQRVKEKLKEKFPNKTIQFAPSSGG
DLEITTHSFNCRGEFFYCYTSDLFNSTYMSNNTGGANITLQCRIK
QIIRMWQGVGQCMYAPPIAGNITCKSNITGLLLTRDGGKEKNDTE
TFRPGGGDMRDNWRSELYKYKVVEIKPLGIAPDKCKRRVVEGGSG
GGGSGGGGSGGAVGIGAVFLGFLGAAGSTMGAASMTLTVQARQLL
SGIVQQQSNLLRAPEAQQHMLQLTVWGIKQLQTRVLAIERYLKDQ
QLLGIWGCSGKLICCTAVPWNASWSNKSYEEIWGNMTWMQWDREI
NNYTNTIYSLLEESQNQQEKNEKDLLALDGGSGGEYGSHGNVATE
LQAYAKLHLERSYDYLLSAAYFNNYQTNRAGFSKLFKKLSDEAWS
KTIDIIKHVTKRGDKMNFDQHSTMKTERKNYTAENHELEALAKAL
DTQKELAERAFYIHREATRNSQHLHDPEIAQYLEEEFIEDHAEKI
RTLAGHTSDLKKFITANNGHDLSLALYVFDEYLQKTV,
SEQ ID NO: 43
Protein sequence, Strain: HT593.1
MRVKEKYQHLWRWGWRWGTMLLGMLMICSATEKLWVTVYYGVPVW
KEATTTLFCASDAKAYETEVHNVWATHACVPTDPNPQEVLLENVT
ENFNMWKNNMVEQMQEDIISLWDQSLKPCVKLTPLCVTLECHDVN
VNGTANNGTTNVTESGVNSSDVTSNNVTNSNWGTMEKGEIKNCSF
NITTNIRDKMQKETAQFYKLDIVPIEDQNKTNNTLYRLINCNTSV
ITQACPKVSFEPIPIHYCTPAGFAILKCNDRNFNGTGPCKNVSTV
QCTHGIKPVVSTQLLLNGSLAEAEVVIRSENFTNNAKTIIIQLNE
TVEINCTRPNNNTSKRISIGPGRAFRATKIIGNIRQAHCNISRAT
WNSTLKKIVAKLREQFGNKTIVFQPSSGGDPEIVMHSFNCGGEFF
YCNTTQLFNSTWNSTEESNSTEEGTITLPCRIKQIINMWQEVGKA
MYAPPIEGQIRCSSNITGLLLTRDGGNNNKTNGTEIFRPGGGDMR
DNWRSELYKYKVVKIEPLGVAPTKAKRRVVQREKRAVGIVGAMFL
GFLGAAGSTMGAASMTLTVQARLLLSGIVQQQNNLLRAIEAQQHL
LQLTVWGIKQLQARVLAVERYLKDQQLLGIWGCSGKLICTTTVPW
NTSWSNKSLSEIWDNMTWMQWEREIDNYTSLIYTLIEESQNQQEK
NEQELLELDKWAGLWNWFEITNWLWYIKIFIMIVGGLVGLRIVFA
VLSIVNRVRQGYSPVSFQTHLPAPRGPDRPEGIEEEGGERDRGRS
VRLVNGFLALIWDDLRSLCLFSYHRLRDLLLIIARIVELLGRRGW
EALKYWWNLLQYWSQELKNSAVNLLDATAIAVAEGTDRIIEVVRR
AFRAILHIPTRIRQGLERALL,
SEQ ID NO: 44
Protein sequence, Strain: HT593.1
(DS.SOSIP.664.sc)
MPMGSLQPLATLYLLGMLVASVLATEKLWVTVYYGVPVWKEATTT
LFCASDAKAYETEVHNVWATHACVPTDPNPQEVLLENVTENFNMW
KNNMVEQMQEDIISLWDQSLKPCVKLTPLCVTLECHDVNVNGTAN
NGTTNVTESGVNSSDVTSNNVTNSNWGTMEKGEIKNCSFNITTNI
RDKMQKETAQFYKLDIVPIEDQNKTNNTLYRLINCNTSVCTQACP
KVSFEPIPIHYCTPAGFAILKCNDRNFNGTGPCKNVSTVQCTHGI
KPVVSTQLLLNGSLAEAEVVIRSENFTNNAKTIIIQLNETVEINC
TRPNNNTSKRISIGPGRAFRATKIIGNIRQAHCNISRATWNSTLK
KIVAKLREQFGNKTIVFQPSSGGDPEIVMHSFNCGGEFFYCNTTQ
LFNSTWNSTEESNSTEEGTITLPCRIKQIINMWQEVGKCMYAPPI
EGQIRCSSNITGLLLTRDGGNNNKTNGTEIFRPGGGDMRDNWRSE
LYKYKVVKIEPLGVAPTKCKRRVVQGGSGGGGSGGGGSGGAVGIV
GAMFLGFLGAAGSTMGAASMTLTVQARLLLSGIVQQQNNLLRAPE
AQQHLLQLTVWGIKQLQARVLAVERYLKDQQLLGIWGCSGKLICC
TTVPWNTSWSNKSLSEIWDNMTWMQWEREIDNYTSLIYTLIEESQ
NQQEKNEQELLELD,
SEQ ID NO: 45
Protein sequence, Strain: HT593.1
(DS.SOSIP.sc + MPER)
MPMGSLQPLATLYLLGMLVASVLATEKLWVTVYYGVPVWKEATTT
LFCASDAKAYETEVHNVWATHACVPTDPNPQEVLLENVTENFNMW
KNNMVEQMQEDIISLWDQSLKPCVKLTPLCVTLECHDVNVNGTAN
NGTTNVTESGVNSSDVTSNNVTNSNWGTMEKGEIKNCSFNITTNI
RDKMQKETAQFYKLDIVPIEDQNKTNNTLYRLINCNTSVCTQACP
KVSFEPIPIHYCTPAGFAILKCNDRNFNGTGPCKNVSTVQCTHGI
KPVVSTQLLLNGSLAEAEVVIRSENFTNNAKTIIIQLNETVEINC
TRPNNNTSKRISIGPGRAFRATKIIGNIRQAHCNISRATWNSTLK
KIVAKLREQFGNKTIVFQPSSGGDPEIVMHSFNCGGEFFYCNTTQ
LFNSTWNSTEESNSTEEGTITLPCRIKQIINMWQEVGKCMYAPPI
EGQIRCSSNITGLLLTRDGGNNNKTNGTEIFRPGGGDMRDNWRSE
LYKYKVVKIEPLGVAPTKCKRRVVQGGSGGGGSGGGGSGGAVGIV
GAMFLGFLGAAGSTMGAASMTLTVQARLLLSGIVQQQNNLLRAPE
AQQHLLQLTVWGIKQLQARVLAVERYLKDQQLLGIWGCSGKLICC
TTVPWNTSWSNKSLSEIWDNMTWMQWEREIDNYTSLIYTLIEESQ
NQQEKNEQELLELDKWAGLWNWFEITNWLWYIKIFIMIVGGLVGL
RIVFAVLSIVNRVRQGYSPVSFQTHLPAPRGPDRPEGIEEEGGER
DRGRSVRLVNGFLALIWDDLRSLCLFSYHRLRDLLLIIARIVELL
GRRGWEALKYWWNLLQYWSQELKNSAVNLLDATAIAVAEGTDRII
EVVRRAFRAILHIPTRIRQGLERALL,
SEQ ID NO: 46
Protein sequence, Strain: HT593.1
(DS.SOSIP.664.sc) + Insect Ferritin Light
Chain,
MPMGSLQPLATLYLLGMLVASVLATEKLWVTVYYGVPVWKEATTT
LFCASDAKAYETEVHNVWATHACVPTDPNPQEVLLENVTENFNMW
KNNMVEQMQEDIISLWDQSLKPCVKLTPLCVTLECHDVNVNGTAN
NGTTNVTESGVNSSDVTSNNVTNSNWGTMEKGEIKNCSFNITTNI
RDKMQKETAQFYKLDIVPIEDQNKTNNTLYRLINCNTSVCTQACP
KVSFEPIPIHYCTPAGFAILKCNDRNFNGTGPCKNVSTVQCTHGI
KPVVSTQLLLNGSLAEAEVVIRSENFTNNAKTIIIQLNETVEINC
TRPNNNTSKRISIGPGRAFRATKIIGNIRQAHCNISRATWNSTLK
KIVAKLREQFGNKTIVFQPSSGGDPEIVMHSFNCGGEFFYCNTTQ
LFNSTWNSTEESNSTEEGTITLPCRIKQIINMWQEVGKCMYAPPI
EGQIRCSSNITGLLLTRDGGNNNKTNGTEIFRPGGGDMRDNWRSE
LYKYKVVKIEPLGVAPTKCKRRVVQGGSGGGGSGGGGSGGAVGIV
GAMFLGFLGAAGSTMGAASMTLTVQARLLLSGIVQQQNNLLRAPE
AQQHLLQLTVWGIKQLQARVLAVERYLKDQQLLGIWGCSGKLICC
TTVPWNTSWSNKSLSEIWDNMTWMQWEREIDNYTSLIYTLIEESQ
NQQEKNEQELLELDGGSGGEYGSHGNVATELQAYAKLHLERSYDY
LLSAAYFNNYQTNRAGFSKLFKKLSDEAWSKTIDIIKHVTKRGDK
MNFDQHSTMKTERKNYTAENHELEALAKALDTQKELAERAFYIHR
EATRNSQHLHDPEIAQYLEEEFIEDHAEKIRTLAGHTSDLKKFIT
ANNGHDLSLALYVFDEYLQKTV
SEQ ID NO: 47
Protein sequence, Strain: KNH1209.18
MRVMGIQRNCQNLLTWGTMILGIIIFCSATDNLWVTVYYGVPVWK
DAETTLFCASDAKAYATEKHNVWATHACVPTDPNPQEIHLENVTE
EFNMWKNNMVEQMHTDIISLWDQSLKPCVKLTPLCVTLSCSNAKV
SYSNATVNNTIQDEIKNCSFNTTTVLRDKRQKVYSLFYRLDIVQI
DNSSSDSSSSEYRLINCNTSAITQACPKVTFEPIPIHYCAPAGFA
ILKCKDEEFNGTGPCKNVSTVQCTHGIKPVVSTQLLLNGSLAKRE
VKIRSENITNNAKNIIVQFVDPVEINCTRPNNNTRKSIHIGPGQA
FYATGDIIGDIRQAHCNVSRSSWNKTLQQVAKQLGTYFKNKTIVF
NTSSGGDPEITTHSFNCAGEFFYCDTSGLFNSSWNDTTWKESNST
GSNDTITLLCRIKQIINMWQRTGQAMYAPPIPGLISCKSNITGII
LTRDGGNSHRTEETFRPGGGDMRDNWRSELYRYKVVQIEPLGVAP
TRARRRVVQREKRAVGIGAVFLGFLGAAGSTMGAASITLTVQARQ
LLSGIVQQQSNLLRAIEAQQHLLKLTVWGIKQLQARVLAVERYLR
DQQLLGIWGCSGKLICTTNVPWNSSWSNKSYNDIWDNMTWLQWDK
EIHNYTQLIYNLIEESQNQQEKNEQDLLALDKWANLWNWFNITNW
LWYIKIFIMVVGGLIGLRIVFAVLSIINRVRQGYSPLSFQTHLPN
PRDLDRPERIEEEGGEQGRDRSIRLVSGFLALAWDDLRSLCLFSY
HRLRDFILIAARTVELLGQSSLKGLRLGWESLKYLWNLLGYWVRE
LKISAVNLVDTIAIAVAGWTDRVIEIGQRIGRAIRHIPRRIRQGL
ERALL,
SEQ ID NO: 48
Protein sequence,
Strain: KNH1209.18 (DS.SOSIP.664.sc)
MPMGSLQPLATLYLLGMLVASVLATDNLWVTVYYGVPVWKDAETT
LFCASDAKAYATEKHNVWATHACVPTDPNPQEIHLENVTEEFNMW
KNNMVEQMHTDIISLWDQSLKPCVKLTPLCVTLSCSNAKVSYSNA
TVNNTIQDEIKNCSFNTTTVLRDKRQKVYSLFYRLDIVQIDNSSS
DSSSSEYRLINCNTSACTQACPKVTFEPIPIHYCAPAGFAILKCK
DEEFNGTGPCKNVSTVQCTHGIKPVVSTQLLLNGSLAKREVKIRS
ENITNNAKNIIVQFVDPVEINCTRPNNNTRKSIHIGPGQAFYATG
DIIGDIRQAHCNVSRSSWNKTLQQVAKQLGTYFKNKTIVFNTSSG
GDPEITTHSFNCAGEFFYCDTSGLFNSSWNDTTWKESNSTGSNDT
ITLLCRIKQIINMWQRTGQCMYAPPIPGLISCKSNITGIILTRDG
GNSHRTEETFRPGGGDMRDNWRSELYRYKVVQIEPLGVAPTRCRR
RVVQGGSGGGGSGGGGSGGAVGIGAVFLGFLGAAGSTMGAASITL
TVQARQLLSGIVQQQSNLLRAPEAQQHLLKLTVWGIKQLQARVLA
VERYLRDQQLLGIWGCSGKLICCTNVPWNSSWSNKSYNDIWDNMT
WLQWDKEIHNYTQLIYNLIEESQNQQEKNEQDLLALD,
SEQ ID NO: 49
Protein sequence, Strain: KNH1209.18
(DS.SOSIP.sc + MPER)
MPMGSLQPLATLYLLGMLVASVLATDNLWVTVYYGVPVWKDAETT
LFCASDAKAYATEKHNVWATHACVPTDPNPQEIHLENVTEEFNMW
KNNMVEQMHTDIISLWDQSLKPCVKLTPLCVTLSCSNAKVSYSNA
TVNNTIQDEIKNCSFNTTTVLRDKRQKVYSLFYRLDIVQIDNSSS
DSSSSEYRLINCNTSACTQACPKVTFEPIPIHYCAPAGFAILKCK
DEEFNGTGPCKNVSTVQCTHGIKPVVSTQLLLNGSLAKREVKIRS
ENITNNAKNIIVQFVDPVEINCTRPNNNTRKSIHIGPGQAFYATG
DIIGDIRQAHCNVSRSSWNKTLQQVAKQLGTYFKNKTIVFNTSSG
GDPEITTHSFNCAGEFFYCDTSGLFNSSWNDTTWKESNSTGSNDT
ITLLCRIKQIINMWQRTGQCMYAPPIPGLISCKSNITGIILTRDG
GNSHRTEETFRPGGGDMRDNWRSELYRYKVVQIEPLGVAPTRCRR
RVVQGGSGGGGSGGGGSGGAVGIGAVFLGFLGAAGSTMGAASITL
TVQARQLLSGIVQQQSNLLRAPEAQQHLLKLTVWGIKQLQARVLA
VERYLRDQQLLGIWGCSGKLICCTNVPWNSSWSNKSYNDIWDNMT
WLQWDKEIHNYTQLIYNLIEESQNQQEKNEQDLLALDKWANLWNW
FNITNWLWYIKIFIMVVGGLIGLRIVFAVLSIINRVRQGYSPLSF
QTHLPNPRDLDRPERIEEEGGEQGRDRSIRLVSGFLALAWDDLRS
LCLFSYHRLRDFILIAARTVELLGQSSLKGLRLGWESLKYLWNLL
GYWVRELKISAVNLVDTIAIAVAGWTDRVIEIGQRIGRAIRHIPR
RIRQGLERALL,
SEQ ID NO: 50
Protein sequence, Strain: KNH1209.18
(DS.SOSIP.664.sc) + Insect Ferritin
Light Chain
MPMGSLQPLATLYLLGMLVASVLATDNLWVTVYYGVPVWKDAETT
LFCASDAKAYATEKHNVWATHACVPTDPNPQEIHLENVTEEFNMW
KNNMVEQMHTDIISLWDQSLKPCVKLTPLCVTLSCSNAKVSYSNA
TVNNTIQDEIKNCSFNTTTVLRDKRQKVYSLFYRLDIVQIDNSSS
DSSSSEYRLINCNTSACTQACPKVTFEPIPIHYCAPAGFAILKCK
DEEFNGTGPCKNVSTVQCTHGIKPVVSTQLLLNGSLAKREVKIRS
ENITNNAKNIIVQFVDPVEINCTRPNNNTRKSIHIGPGQAFYATG
DIIGDIRQAHCNVSRSSWNKTLQQVAKQLGTYFKNKTIVFNTSSG
GDPEITTHSFNCAGEFFYCDTSGLFNSSWNDTTWKESNSTGSNDT
ITLLCRIKQIINMWQRTGQCMYAPPIPGLISCKSNITGIILTRDG
GNSHRTEETFRPGGGDMRDNWRSELYRYKVVQIEPLGVAPTRCRR
RVVQGGSGGGGSGGGGSGGAVGIGAVFLGFLGAAGSTMGAASITL
TVQARQLLSGIVQQQSNLLRAPEAQQHLLKLTVWGIKQLQARVLA
VERYLRDQQLLGIWGCSGKLICCTNVPWNSSWSNKSYNDIWDNMT
WLQWDKEIHNYTQLIYNLIEESQNQQEKNEQDLLALDGGSGGEYG
SHGNVATELQAYAKLHLERSYDYLLSAAYFNNYQTNRAGFSKLFK
KLSDEAWSKTIDIIKHVTKRGDKMNFDQHSTMKTERKNYTAENHE
LEALAKALDTQKELAERAFYIHREATRNSQHLHDPEIAQYLEEEF
IEDHAEKIRTLAGHTSDLKKFITANNGHDLSLALYVFDEYLQKTV,
SEQ ID NO: 51
Protein sequence, Strain: MB539.2B7
MRVMGTQRNCQHLLTWGTLILGIIIICSTAENLWVTVYYGVPVWR
DADTTLFCASDAKAYETEKHNVWATHACVPTDPNPQEIDLKNVTE
EFNMWKNNMVEQMHTDIISLWDQSLKPCVKLTPLCVTLNCSNANV
TSENSTIMGDREEIKNCSFNMTTELRDKRQKVYSLFYRLDVVQIN
ENQGNSSNNNYSEYRLINCNTSAITQACPKVSFEPIPIHYCAPAG
FAILKCKDEEFNGTGPCKNVSTVQCTHGIKPVVSTQLLLNGSTAE
KEIKIRSENITNNAKIIIVQLVKPVIINCTRPNNNTRRSVHIGPG
QAFYATGDIIGNIRQAYCTVNRTDWNNTLQQVAKQLGKHFENKTI
IFTKSSGGDLEITTHSFNCGGEFFYCNTSSLFNSTWSHNNSTLLG
SNSTESNETITLPCRIKQIVNMWQRTGQAMYAPPIKGVIMCVSNI
TGLILTRDGGNDNSTNENETFRPGGGDMRDNWRSELYKYKVVQIE
PLGVAPTRAKRRVVEREKRAVGIGAVFLGFLGAAGSTMGAASITL
TVQARQLLSGIVRQQSNLLRAIEAQQHLLKLTVWGIKQLQARVLA
VERYLRDQQLLGIWGCSGKLICTTSVPWNSSWSNKSLDEIWENMT
WLQWEKEINNYTGLIYSLLEESQNQQEKNEQDLLALDKWANLWTW
FGISNWLWYIRIFIIIVGGLIGLRIVFAVLSVVNRVRQGYSPLSF
QIHPPNPGGLDRPGRIEEEGGEQGRDRSIRLVSGFLALAWDDLRS
LCLFSYHRLRDFILIAARTVELLGHSSLKGLRLGWEGLKYLWNLL
AYWGRELKISAISLVDNIAIVVAGWTDRVIEIGQGIGRAILHIPR
RIRQGFERALL,
SEQ ID NO: 52
Protein sequence, Strain: MB539.2B7
(DS.SOSIP.664.sc)
MPMGSLQPLATLYLLGMLVASVLAAENLWVTVYYGVPVWRDADTT
LFCASDAKAYETEKHNVWATHACVPTDPNPQEIDLKNVTEEFNMW
KNNMVEQMHTDIISLWDQSLKPCVKLTPLCVTLNCSNANVTSENS
TIMGDREEIKNCSFNMTTELRDKRQKVYSLFYRLDVVQINENQGN
SSNNNYSEYRLINCNTSACTQACPKVSFEPIPIHYCAPAGFAILK
CKDEEFNGTGPCKNVSTVQCTHGIKPVVSTQLLLNGSTAEKEIKI
RSENITNNAKIIIVQLVKPVIINCTRPNNNTRRSVHIGPGQAFYA
TGDIIGNIRQAYCTVNRTDWNNTLQQVAKQLGKHFENKTIIFTKS
SGGDLEITTHSFNCGGEFFYCNTSSLFNSTWSHNNSTLLGSNSTE
SNETITLPCRIKQIVNMWQRTGQCMYAPPIKGVIMCVSNITGLIL
TRDGGNDNSTNENETFRPGGGDMRDNWRSELYKYKVVQIEPLGVA
PTRCKRRVVEGGSGGGGSGGGGSGGAVGIGAVFLGFLGAAGSTMG
AASITLTVQARQLLSGIVRQQSNLLRAPEAQQHLLKLTVWGIKQL
QARVLAVERYLRDQQLLGIWGCSGKLICCTSVPWNSSWSNKSLDE
IWENMTWLQWEKEINNYTGLIYSLLEESQNQQEKNEQDLLALD,
SEQ ID NO: 53
Protein sequence,
Strain: MB539.2B7 (DS.SOSIP.sc + MPER)
MPMGSLQPLATLYLLGMLVASVLAAENLWVTVYYGVPVWRDADTT
LFCASDAKAYETEKHNVWATHACVPTDPNPQEIDLKNVTEEFNMW
KNNMVEQMHTDIISLWDQSLKPCVKLTPLCVTLNCSNANVTSENS
TIMGDREEIKNCSFNMTTELRDKRQKVYSLFYRLDVVQINENQGN
SSNNNYSEYRLINCNTSACTQACPKVSFEPIPIHYCAPAGFAILK
CKDEEFNGTGPCKNVSTVQCTHGIKPVVSTQLLLNGSTAEKEIKI
RSENITNNAKIIIVQLVKPVIINCTRPNNNTRRSVHIGPGQAFYA
TGDIIGNIRQAYCTVNRTDWNNTLQQVAKQLGKHFENKTIIFTKS
SGGDLEITTHSFNCGGEFFYCNTSSLFNSTWSHNNSTLLGSNSTE
SNETITLPCRIKQIVNMWQRTGQCMYAPPIKGVIMCVSNITGLIL
TRDGGNDNSTNENETFRPGGGDMRDNWRSELYKYKVVQIEPLGVA
PTRCKRRVVEGGSGGGGSGGGGSGGAVGIGAVFLGFLGAAGSTMG
AASITLTVQARQLLSGIVRQQSNLLRAPEAQQHLLKLTVWGIKQL
QARVLAVERYLRDQQLLGIWGCSGKLICCTSVPWNSSWSNKSLDE
IWENMTWLQWEKEINNYTGLIYSLLEESQNQQEKNEQDLLALDKW
ANLWTWFGISNWLWYIRIFIIIVGGLIGLRIVFAVLSVVNRVRQG
YSPLSFQIHPPNPGGLDRPGRIEEEGGEQGRDRSIRLVSGFLALA
WDDLRSLCLFSYHRLRDFILIAARTVELLGHSSLKGLRLGWEGLK
YLWNLLAYWGRELKISAISLVDNIAIVVAGWTDRVIEIGQGIGRA
ILHIPRRIRQGFERALL,
SEQ ID NO: 54
Protein sequence, Strain: MB539.2B7
(DS.SOSIP.664.sc) + Insect Ferritin
Heavy Chain
MPMGSLQPLATLYLLGMLVASVLAAENLWVTVYYGVPVWRDADTT
LFCASDAKAYETEKHNVWATHACVPTDPNPQEIDLKNVTEEFNMW
KNNMVEQMHTDIISLWDQSLKPCVKLTPLCVTLNCSNANVTSENS
TIMGDREEIKNCSFNMTTELRDKRQKVYSLFYRLDVVQINENQGN
SSNNNYSEYRLINCNTSACTQACPKVSFEPIPIHYCAPAGFAILK
CKDEEFNGTGPCKNVSTVQCTHGIKPVVSTQLLLNGSTAEKEIKI
RSENITNNAKIIIVQLVKPVIINCTRPNNNTRRSVHIGPGQAFYA
TGDIIGNIRQAYCTVNRTDWNNTLQQVAKQLGKHFENKTIIFTKS
SGGDLEITTHSFNCGGEFFYCNTSSLFNSTWSHNNSTLLGSNSTE
SNETITLPCRIKQIVNMWQRTGQCMYAPPIKGVIMCVSNITGLIL
TRDGGNDNSTNENETFRPGGGDMRDNWRSELYKYKVVQIEPLGVA
PTRCKRRVVEGGSGGGGSGGGGSGGAVGIGAVFLGFLGAAGSTMG
AASITLTVQARQLLSGIVRQQSNLLRAPEAQQHLLKLTVWGIKQL
QARVLAVERYLRDQQLLGIWGCSGKLICCTSVPWNSSWSNKSLDE
IWENMTWLQWEKEINNYTGLIYSLLEESQNQQEKNEQDLLALDGG
SGGRSCRNSMRQQIQMEVGASLQYLAMGAHFSKDVVNRPGFAQLF
FDAASEEREHAMKLIEYLLMRGELTNDVSSLLQVRPPTRSSWKGG
VEALEHALSMESDVTKSIRNVIKACEDDSEFNDYHLVDYLTGDFL
EEQYKGQRDLAGKASTLKKLMDRHEALGEFIFDKKLLGIDV,
SEQ ID NO: 55
Protein sequence, Strain: RHPA.7
MRVMGIRKNYQHLWKWGTMLLWLLMICSAADQLWVTVYYGVPVWK
EANTTLFCASDAKAYDTEAHNVWATHACVPTDPNPQEVVLENVTE
NFNMWKNHMVEQMHEDIISLWDQSLKPCVKLTPLCVTLNCTDLVN
SNITRVDNTTEKEMKNCSFNVTSGIRDKVQKEYALLYKLDIVQID
NDNTSHRDNTSYRLISCNTSVITQACPKISFEPIPIHFCAPAGFA
ILKCNDKKFNGTGPCTNVSTVQCTHGIRPVVSTQLLLNGSLAEEE
VVIRSENFTNNVKNIIVQLNESVQINCTRHNNNTRKSINIGPGRA
FYATGKIIGDIRQAHCNISREKWQNTLKQIVKKLREQFKNKTIAF
APSSGGDPEIVMHSFNCNGEFFYCNTTKLFTSTWNSTWNSTWNNT
EGSNSTVITLPCRIRQIINMWQEVGKAMYAPPIQGQIKCSSNITG
LLLTRDGGVDTTKETFRPGGGNMKDNWRSELYKYKVVRIEPLGVA
PTKAKRRVVQREKRAVGIGAMFLGFLGAAGSTMGAASITLTVQAR
LLLSGIVQQQSNLLRAIEAQQHLLQLTVWGIKQLQARVLAVERYL
KDQQLLGIWGCSGKLICTTAVPWNASWSNKSQDTIWGNMTWMQWE
REIDNYTDLIYNLLEESQNQQEKNEQELLALDKWASLWSWFSITH
WLWYIKMFIMIVGGLVGLRIVFAVLSIVNRVRQGYSPLSFQTRFP
APRGPDRPEGIEEEGGERDRDRSGRSADGFLVLVWVDLRNLCLFS
YHRLRDLLLIVTRTVELLGRRGWEALKYWWNLLQYWSQELKKSAV
SLLDAIAIAVAEGTDRIIELLQRIFRAFLHIPTRIRQGLERALQ,
SEQ ID NO: 56
Protein sequence, Strain: RHPA.7
(DS.SOSIP.664.sc)
MPMGSLQPLATLYLLGMLVASVLAADQLWVTVYYGVPVWKEANTT
LFCASDAKAYDTEAHNVWATHACVPTDPNPQEVVLENVTENFNMW
KNHMVEQMHEDIISLWDQSLKPCVKLTPLCVTLNCTDLVNSNITR
VDNTTEKEMKNCSFNVTSGIRDKVQKEYALLYKLDIVQIDNDNTS
HRDNTSYRLISCNTSVCTQACPKISFEPIPIHFCAPAGFAILKCN
DKKFNGTGPCTNVSTVQCTHGIRPVVSTQLLLNGSLAEEEVVIRS
ENFTNNVKNIIVQLNESVQINCTRHNNNTRKSINIGPGRAFYATG
KIIGDIRQAHCNISREKWQNTLKQIVKKLREQFKNKTIAFAPSSG
GDPEIVMHSFNCNGEFFYCNTTKLFTSTWNSTWNSTWNNTEGSNS
TVITLPCRIRQIINMWQEVGKCMYAPPIQGQIKCSSNITGLLLTR
DGGVDTTKETFRPGGGNMKDNWRSELYKYKVVRIEPLGVAPTKCK
RRVVQGGSGGGGSGGGGSGGAVGIGAMFLGFLGAAGSTMGAASIT
LTVQARLLLSGIVQQQSNLLRAPEAQQHLLQLTVWGIKQLQARVL
AVERYLKDQQLLGIWGCSGKLICCTAVPWNASWSNKSQDTIWGNM
TWMQWEREIDNYTDLIYNLLEESQNQQEKNEQELLALD,
SEQ ID NO: 57
Protein sequence, Strain: RHPA.7
(DS.SOSIP.sc + MPER)
MPMGSLQPLATLYLLGMLVASVLAADQLWVTVYYGVPVWKEANTT
LFCASDAKAYDTEAHNVWATHACVPTDPNPQEVVLENVTENFNMW
KNHMVEQMHEDIISLWDQSLKPCVKLTPLCVTLNCTDLVNSNITR
VDNTTEKEMKNCSFNVTSGIRDKVQKEYALLYKLDIVQIDNDNTS
HRDNTSYRLISCNTSVCTQACPKISFEPIPIHFCAPAGFAILKCN
DKKFNGTGPCTNVSTVQCTHGIRPVVSTQLLLNGSLAEEEVVIRS
ENFTNNVKNIIVQLNESVQINCTRHNNNTRKSINIGPGRAFYATG
KIIGDIRQAHCNISREKWQNTLKQIVKKLREQFKNKTIAFAPSSG
GDPEIVMHSFNCNGEFFYCNTTKLFTSTWNSTWNSTWNNTEGSNS
TVITLPCRIRQIINMWQEVGKCMYAPPIQGQIKCSSNITGLLLTR
DGGVDTTKETFRPGGGNMKDNWRSELYKYKVVRIEPLGVAPTKCK
RRVVQGGSGGGGSGGGGSGGAVGIGAMFLGFLGAAGSTMGAASIT
LTVQARLLLSGIVQQQSNLLRAPEAQQHLLQLTVWGIKQLQARVL
AVERYLKDQQLLGIWGCSGKLICCTAVPWNASWSNKSQDTIWGNM
TWMQWEREIDNYTDLIYNLLEESQNQQEKNEQELLALDKWASLWS
WFSITHWLWYIKMFIMIVGGLVGLRIVFAVLSIVNRVRQGYSPLS
FQTRFPAPRGPDRPEGIEEEGGERDRDRSGRSADGFLVLVWVDLR
NLCLFSYHRLRDLLLIVTRTVELLGRRGWEALKYWWNLLQYWSQE
LKKSAVSLLDAIAIAVAEGTDRIIELLQRIFRAFLHIPTRIRQGL
ERALQ,
SEQ ID NO: 58
Protein sequence, Strain: RHPA.7
(DS.SOSIP.664.sc) + Insect Ferritin Heavy
Chain
MPMGSLQPLATLYLLGMLVASVLAADQLWVTVYYGVPVWKEANTTL
FCASDAKAYDTEAHNVWATHACVPTDPNPQEVVLENVTENFNMWK
NHMVEQMHEDIISLWDQSLKPCVKLTPLCVTLNCTDLVNSNITRV
DNTTEKEMKNCSFNVTSGIRDKVQKEYALLYKLDIVQIDNDNTSH
RDNTSYRLISCNTSVCTQACPKISFEPIPIHFCAPAGFAILKCND
KKFNGTGPCTNVSTVQCTHGIRPVVSTQLLLNGSLAEEEVVIRSE
NFTNNVKNIIVQLNESVQINCTRHNNNTRKSINIGPGRAFYATGK
IIGDIRQAHCNISREKWQNTLKQIVKKLREQFKNKTIAFAPSSGG
DPEIVMHSFNCNGEFFYCNTTKLFTSTWNSTWNSTWNNTEGSNST
VITLPCRIRQIINMWQEVGKCMYAPPIQGQIKCSSNITGLLLTRD
GGVDTTKETFRPGGGNMKDNWRSELYKYKVVRIEPLGVAPTKCKR
RVVQGGSGGGGSGGGGSGGAVGIGAMFLGFLGAAGSTMGAASITL
TVQARLLLSGIVQQQSNLLRAPEAQQHLLQLTVWGIKQLQARVLA
VERYLKDQQLLGIWGCSGKLICCTAVPWNASWSNKSQDTIWGNMT
WMQWEREIDNYTDLIYNLLEESQNQQEKNEQELLALDGGSGGRSC
RNSMRQQIQMEVGASLQYLAMGAHFSKDVVNRPGFAQLFFDAASE
EREHAMKLIEYLLMRGELTNDVSSLLQVRPPTRSSWKGGVEALEH
ALSMESDVTKSIRNVIKACEDDSEFNDYHLVDYLTGDFLEEQYKG
QRDLAGKASTLKKLMDRHEALGEFIFDKKLLGIDV,
SEQ ID NO: 59
Protein sequence, Strain: RW020.2
MRVRGIQTSWQNLWRWGTMILGMLMIYSAAENLWVTVYYGVPVWK
DAETTLFCASDAKAYDTEVHNVWATHACVPTDPNPQEIHLENVTE
DFNMWKNNMVEQMHTDIISLWDQSLKPCVKLTPLCVTLDCNATAS
NVTNEMRNCSFNITTELKDKKQQVYSLFYKLDVVQINEKNETDKY
RLINCNTSAITQACPKVSFEPIPIHYCAPAGFAVLKCKDTEFNGT
GPCKNVSTVQCTHGIRPVISTQLLLNGSLAEEGIQIRSENITNNA
KTIIVQLDKAVKINCTRPNNNTRKGVRIGPGQAFYATGGIIGDIR
QAHCNVSRAKWNDTLRGVAKKLREHFKNKTIIFEKSSGGDIEITT
HSFNCGGEFFYCSTSGLFNSTWESNSTESNNTTSNDTITLTCRIK
QIINMWQKVGQAMYAPPIQGVIRCESNITGLLLTRDGGNNSTNEI
FRPGGGNMRDNWRSELYKYKVVKIEPLGVAPSRAKRRVVEREKRA
VGIGAVFLGFLGAAGSTMGAASITLTAQARQLLSGIVQQQSNLLR
AIEAQQHMLKLTVWGIKQLQARVLAVERYLKDQQLLGIWGCSGKL
ICTTNVPWNSSWSNKSMNEIWDNMTWLQWDKEISNYTQIIYNLIE
ESQNQQEKNEQDLLALDKWASLWNWFDISRWLWYIKIFIMIVGGL
IGLRIVFAVLSVINRVRQGYSPLSFQIRTPNPKEPDRLGRIDGEG
GEQDRDRSIRLVSGFLALAWDDLRSLCLFSYHRLRDFISIAARTV
ELLGHSSLKGLRLGWEGLKYLWNLLLYWGRELKTSAVNLVDTIAI
AVAGWADRVMEVGQRIFRAILNIPRRIRQGLERGLL,
SEQ ID NO: 60
Protein sequence,
Strain: RW020.2 (DS.SOSIP.664.sc)
MPMGSLQPLATLYLLGMLVASVLAENLWVTVYYGVPVWKDAETTL
FCASDAKAYDTEVHNVWATHACVPTDPNPQEIHLENVTEDFNMWK
NNMVEQMHTDIISLWDQSLKPCVKLTPLCVTLDCNATASNVTNEM
RNCSFNITTELKDKKQQVYSLFYKLDVVQINEKNETDKYRLINCN
TSACTQACPKVSFEPIPIHYCAPAGFAVLKCKDTEFNGTGPCKNV
STVQCTHGIRPVISTQLLLNGSLAEEGIQIRSENITNNAKTIIVQ
LDKAVKINCTRPNNNTRKGVRIGPGQAFYATGGIIGDIRQAHCNV
SRAKWNDTLRGVAKKLREHFKNKTIIFEKSSGGDIEITTHSFNCG
GEFFYCSTSGLFNSTWESNSTESNNTTSNDTITLTCRIKQIINMW
QKVGQCMYAPPIQGVIRCESNITGLLLTRDGGNNSTNEIFRPGGG
NMRDNWRSELYKYKVVKIEPLGVAPSRCKRRVVEGGSGGGGSGGG
GSGGAVGIGAVFLGFLGAAGSTMGAASITLTAQARQLLSGIVQQQ
SNLLRAPEAQQHMLKLTVWGIKQLQARVLAVERYLKDQQLLGIWG
CSGKLICCTNVPWNSSWSNKSMNEIWDNMTWLQWDKEISNYTQII
YNLIEESQNQQEKNEQDLLALD,
SEQ ID NO: 61
Protein sequence,
Strain: RW020.2 (DS.SOSIP.sc + MPER)
MPMGSLQPLATLYLLGMLVASVLAENLWVTVYYGVPVWKDAETTL
FCASDAKAYDTEVHNVWATHACVPTDPNPQEIHLENVTEDFNMWK
NNMVEQMHTDIISLWDQSLKPCVKLTPLCVTLDCNATASNVTNEM
RNCSFNITTELKDKKQQVYSLFYKLDVVQINEKNETDKYRLINCN
TSACTQACPKVSFEPIPIHYCAPAGFAVLKCKDTEFNGTGPCKNV
STVQCTHGIRPVISTQLLLNGSLAEEGIQIRSENITNNAKTIIVQ
LDKAVKINCTRPNNNTRKGVRIGPGQAFYATGGIIGDIRQAHCNV
SRAKWNDTLRGVAKKLREHFKNKTIIFEKSSGGDIEITTHSFNCG
GEFFYCSTSGLFNSTWESNSTESNNTTSNDTITLTCRIKQIINMW
QKVGQCMYAPPIQGVIRCESNITGLLLTRDGGNNSTNEIFRPGGG
NMRDNWRSELYKYKVVKIEPLGVAPSRCKRRVVEGGSGGGGSGGG
GSGGAVGIGAVFLGFLGAAGSTMGAASITLTAQARQLLSGIVQQQ
SNLLRAPEAQQHMLKLTVWGIKQLQARVLAVERYLKDQQLLGIWG
CSGKLICCTNVPWNSSWSNKSMNEIWDNMTWLQWDKEISNYTQII
YNLIEESQNQQEKNEQDLLALDKWASLWNWFDISRWLWYIKIFIM
IVGGLIGLRIVFAVLSVINRVRQGYSPLSFQIRTPNPKEPDRLGR
IDGEGGEQDRDRSIRLVSGFLALAWDDLRSLCLFSYHRLRDFISI
AARTVELLGHSSLKGLRLGWEGLKYLWNLLLYWGRELKTSAVNLV
DTIAIAVAGWADRVMEVGQRIFRAILNIPRRIRQGLERGLL,
SEQ ID NO: 62
Protein sequence, Strain: RW020.2
(DS.SOSIP.664.sc) + Insect Ferritin Light
Chain
MPMGSLQPLATLYLLGMLVASVLAENLWVTVYYGVPVWKDAETTL
FCASDAKAYDTEVHNVWATHACVPTDPNPQEIHLENVTEDFNMWK
NNMVEQMHTDIISLWDQSLKPCVKLTPLCVTLDCNATASNVTNEM
RNCSFNITTELKDKKQQVYSLFYKLDVVQINEKNETDKYRLINCN
TSACTQACPKVSFEPIPIHYCAPAGFAVLKCKDTEFNGTGPCKNV
STVQCTHGIRPVISTQLLLNGSLAEEGIQIRSENITNNAKTIIVQ
LDKAVKINCTRPNNNTRKGVRIGPGQAFYATGGIIGDIRQAHCNV
SRAKWNDTLRGVAKKLREHFKNKTIIFEKSSGGDIEITTHSFNCG
GEFFYCSTSGLFNSTWESNSTESNNTTSNDTITLTCRIKQIINMW
QKVGQCMYAPPIQGVIRCESNITGLLLTRDGGNNSTNEIFRPGGG
NMRDNWRSELYKYKVVKIEPLGVAPSRCKRRVVEGGSGGGGSGGG
GSGGAVGIGAVFLGFLGAAGSTMGAASITLTAQARQLLSGIVQQQ
SNLLRAPEAQQHMLKLTVWGIKQLQARVLAVERYLKDQQLLGIWG
CSGKLICCTNVPWNSSWSNKSMNEIWDNMTWLQWDKEISNYTQII
YNLIEESQNQQEKNEQDLLALDGGSGGEYGSHGNVATELQAYAKL
HLERSYDYLLSAAYFNNYQTNRAGFSKLFKKLSDEAWSKTIDIIK
HVTKRGDKMNFDQHSTMKTERKNYTAENHELEALAKALDTQKELA
ERAFYIHREATRNSQHLHDPEIAQYLEEEFIEDHAEKIRTLAGHT
SDLKKFITANNGHDLSLALYVFDEYLQKTV,
SEQ ID NO: 63
Protein sequence, Strain: SO18.18
MRVRGISRNWQQWWIWGVLGFWLLMSYSVLGNLWVTVYYGVP
VWKEAKTTLFC
ASDAKAYEREVHNVWATHACVPTDPNPQEMVLENVTENFNMWKND
MVDQMHEDIISLWDQSLKPCVKLTPLCVTLNCTNASVNATYNGEM
KNCSFNATTAIRDKKQQVRALFYSLDIVPLEGNNSSYRLISCNTS
AITQACPKVSFDPIPIHYCTPAGYAILKCNDEKFNGTGPCHNVST
VQCTHGIKPVVSTQLLLNGSLAEKEIIIRSENLTNNAKTIIVHLN
KAVEIVCVRPNNNTRKSIRIGPGQTFYANDIIGDIRQAHCNISES
KWNDTLRQVGAKLAEHFNNNTIRFEPSSGGDLEITTHSFNCRGEF
FYCNTSGLFNGTYNHTDTGGNSTNITLPCRIKQIINMWQEVGRAI
YAPPVEGNIICISNITGLLLLRDGGHNSTNETFRPGGGDMRDNWR
SELYKYKVVEIKPLGVAPTEAKRRVVEREKRAVGIGAMFLGFLGA
AGSTMGAASITLTVQARQLLSGIVQQQSNLLRAIEAQQHMLQLTV
WGIKQLQARVLSIERYLKDQQLLGLWGCSGKLICTTSVPWNHSWS
NKSQKDIWENMTWMQWDREINNYTNTIYSLLEESQSQQEKNEKDL
LALDNWNNLWNWFSITKWLWYIKIFIIIVGGLIGLRIIFAVLSIV
NRVRQGYSPLSLQTLIPSPRGPDRLGRIEEEGGEQDKDRSIRLVS
GFLSLAWDDLRSLCLFSYHRLRDFLLVTARAVELLGRSSLKGLQK
GWEALKYLGNLVQYWGLELKKSVISLIDIIAIAVAEGTDRIIEVI
QRICRAIRNIPTRIRQGFETALL,
SEQ ID NO: 64
Protein sequence,
Strain: SO18.18 (DS.SOSIP.664.sc)
MPMGSLQPLATLYLLGMLVASVLANLWVTVYYGVPVWKEAKTTLF
CASDAKAYEREVHNVWATHACVPTDPNPQEMVLENVTENFNMWKN
DMVDQMHEDIISLWDQSLKPCVKLTPLCVTLNCTNASVNATYNGE
MKNCSFNATTAIRDKKQQVRALFYSLDIVPLEGNNSSYRLISCNT
SACTQACPKVSFDPIPIHYCTPAGYAILKCNDEKFNGTGPCHNVS
TVQCTHGIKPVVSTQLLLNGSLAEKEIIIRSENLTNNAKTIIVHL
NKAVEIVCVRPNNNTRKSIRIGPGQTFYANDIIGDIRQAHCNISE
SKWNDTLRQVGAKLAEHFNNNTIRFEPSSGGDLEITTHSFNCRGE
FFYCNTSGLFNGTYNHTDTGGNSTNITLPCRIKQIINMWQEVGRC
IYAPPVEGNIICISNITGLLLLRDGGHNSTNETFRPGGGDMRDNW
RSELYKYKVVEIKPLGVAPTECKRRVVEGGSGGGGSGGGGSGGAV
GIGAMFLGFLGAAGSTMGAASITLTVQARQLLSGIVQQQSNLLRA
PEAQQHMLQLTVWGIKQLQARVLSIERYLKDQQLLGLWGCSGKLI
CCTSVPWNHSWSNKSQKDIWENMTWMQWDREINNYTNTIYSLLEE
SQSQQEKNEKDLLALD,
SEQ ID NO: 65
Protein sequence, Strain: SO18.18
(DS.SOSIP.664.sc + MPER)
MPMGSLQPLATLYLLGMLVASVLANLWVTVYYGVPVWKEAKTTLF
CASDAKAYEREVHNVWATHACVPTDPNPQEMVLENVTENFNMWKN
DMVDQMHEDIISLWDQSLKPCVKLTPLCVTLNCTNASVNATYNGE
MKNCSFNATTAIRDKKQQVRALFYSLDIVPLEGNNSSYRLISCNT
SACTQACPKVSFDPIPIHYCTPAGYAILKCNDEKFNGTGPCHNVS
TVQCTHGIKPVVSTQLLLNGSLAEKEIIIRSENLTNNAKTIIVHL
NKAVEIVCVRPNNNTRKSIRIGPGQTFYANDIIGDIRQAHCNISE
SKWNDTLRQVGAKLAEHFNNNTIRFEPSSGGDLEITTHSFNCRGE
FFYCNTSGLFNGTYNHTDTGGNSTNITLPCRIKQIINMWQEVGRC
IYAPPVEGNIICISNITGLLLLRDGGHNSTNETFRPGGGDMRDNW
RSELYKYKVVEIKPLGVAPTECKRRVVEGGSGGGGSGGGGSGGAV
GIGAMFLGFLGAAGSTMGAASITLTVQARQLLSGIVQQQSNLLRA
PEAQQHMLQLTVWGIKQLQARVLSIERYLKDQQLLGLWGCSGKLI
CCTSVPWNHSWSNKSQKDIWENMTWMQWDREINNYTNTIYSLLEE
SQSQQEKNEKDLLALDNWNNLWNWFSITKWLWYIKIFIIIVGGLI
GLRIIFAVLSIVNRVRQGYSPLSLQTLIPSPRGPDRLGRIEEEGG
EQDKDRSIRLVSGFLSLAWDDLRSLCLFSYHRLRDFLLVTARAVE
LLGRSSLKGLQKGWEALKYLGNLVQYWGLELKKSVISLIDIIAIA
VAEGTDRIIEVIQRICRAIRNIPTRIRQGFETALL,
SEQ ID NO: 66
Protein sequence, Strain: SOI 8.18
(DS.SOSIP.664.sc) + Insect Ferritin Light
Chain
MPMGSLQPLATLYLLGMLVASVLANLWVTVYYGVPVWKEAKTTLF
CASDAKAYEREVHNVWATHACVPTDPNPQEMVLENVTENFNMWKN
DMVDQMHEDIISLWDQSLKPCVKLTPLCVTLNCTNASVNATYNGE
MKNCSFNATTAIRDKKQQVRALFYSLDIVPLEGNNSSYRLISCNT
SACTQACPKVSFDPIPIHYCTPAGYAILKCNDEKFNGTGPCHNVS
TVQCTHGIKPVVSTQLLLNGSLAEKEIIIRSENLTNNAKTIIVHL
NKAVEIVCVRPNNNTRKSIRIGPGQTFYANDIIGDIRQAHCNISE
SKWNDTLRQVGAKLAEHFNNNTIRFEPSSGGDLEITTHSFNCRGE
FFYCNTSGLFNGTYNHTDTGGNSTNITLPCRIKQIINMWQEVGRC
IYAPPVEGNIICISNITGLLLLRDGGHNSTNETFRPGGGDMRDNW
RSELYKYKVVEIKPLGVAPTECKRRVVEGGSGGGGSGGGGSGGAV
GIGAMFLGFLGAAGSTMGAASITLTVQARQLLSGIVQQQSNLLRA
PEAQQHMLQLTVWGIKQLQARVLSIERYLKDQQLLGLWGCSGKLI
CCTSVPWNHSWSNKSQKDIWENMTWMQWDREINNYTNTIYSLLEE
SQSQQEKNEKDLLALDGGSGGEYGSHGNVATELQAYAKLHLERSY
DYLLSAAYFNNYQTNRAGFSKLFKKLSDEAWSKTIDIIKHVTKRG
DKMNFDQHSTMKTERKNYTAENHELEALAKALDTQKELAERAFYI
HREATRNSQHLHDPEIAQYLEEEFIEDHAEKIRTLAGHTSDLKKF
ITANNGHDLSLALYVFDEYLQKTV,
SEQ ID NO: 67
Protein sequence, Strain: 286.36
(DS.SOSIP.664.sc) 2A DU172.17
(DS.SOSIP.sc)
MPMGSLQPLATLYLLGMLVASVLAGEDLWVTVYYGVPVWKEANPT
LFCASDAKAYKTEMHNVWATHACVPTDPNPQEMVLENVTEDFNMW
KNGMVEQMHQDIISLWDQSLKPCVKLTPLCVTLNCTEVTRSSNGT
INNNSTEMKNCSFNVTTDLRDKKKKEHALFYRLDIVPLDETNGTS
SEYRLINCNTSTCTQACPKVSFDPIPIHYCAPAGYAILKCKDKKF
NGTGPCKNVSTVQCTHGIKPVVSTQLLLNGSIAEGEIIIRSENLT
NNAKIIIVQLNVTVEINCTRPNNNTRRSIRIGPGQTFYATGEIIG
DIRQAHCNISREKWNRTLQKVEKKLEELFPNKTIHFTSSSGGDLE
ITTHSFNCMGEFFYCNTSALFNNNNDSTNSNITLPCRIRQFINMW
QEVGRCMYAPPIQGVITCKSNVTGLLLTRDGGIINDTEIFRPGGG
DMRDNWRSELYKYKVVEIKPLGIAPTTCKRRVVEGGSGGGGSGGG
GSGGAVGIGAVFLGFLGAAGSTMGAASITLTAQARQLLSGIVQQQ
SNLLRAPEAQQHMLQLTVWGIKQLQTRVLAIERYLKDQQLLGIWG
CSGKLICCTAVPWNGSWSNKSQDEIWHNMTWMQWDKEINNYTNII
YGLLEVSQNQQEKNEQDLLALDGSGATNFSLLKQAGDVEENPGPG
SGMKAKLLVLLCTFTATYAGNLWVTVYYGVPVWKEAKTTLFCASD
AKAHKEEVHNIWATHACVPTDPNPQEIVLKNVTENFNMWKNDMVD
QMHEDIISLWDQSLKPCVKLTPLCVTLNCSDVKIKGTNATYNNAT
YNNNNTISDMKNCSFNTTTEITDKKKKEYALFYKLDVVALDGKET
NSTNSSEYRLINCNTSACTQACPKVSFDPIPIHYCAPAGYAILKC
NNKTFNGTGPCNNVSTVQCTHGIKPVVSTQLLLNGSLAEEEVVIR
FENLTNNAKIIIVHLNESVEINCTRPSNNTRKSVRIGPGQTFFAT
GDIIGDIRQAHCNISRKKWNTTLQRVKEKLKEKFPNKTIQFAPSS
GGDLEITTHSFNCRGEFFYCYTSDLFNSTYMSNNTGGANITLQCR
IKQIIRMWQGVGQCMYAPPIAGNITCKSNITGLLLTRDGGKEKND
TETFRPGGGDMRDNWRSELYKYKVVEIKPLGIAPDKCKRRVVEGG
SGGGGSGGGGSGGAVGIGAVFLGFLGAAGSTMGAASMTLTVQARQ
LLSGIVQQQSNLLRAPEAQQHMLQLTVWGIKQLQTRVLAIERYLK
DQQLLGIWGCSGKLICCTAVPWNASWSNKSYEEIWGNMTWMQWDR
EINNYTNTIYSLLEESQNQQEKNEKDLLALD,
SEQ ID NO: 68
Protein sequence, Strain: MB539.2B7
(DS.SOSIP.664.sc) 2A KNH1209.18
(DS.SOSIP.664.sc)
MPMGSLQPLATLYLLGMLVASVLAAENLWVTVYYGVPVWRDADTT
LFCASDAKAYETEKHNVWATHACVPTDPNPQEIDLKNVTEEFNMW
KNNMVEQMHTDIISLWDQSLKPCVKLTPLCVTLNCSNANVTSENS
TIMGDREEIKNCSFNMTTELRDKRQKVYSLFYRLDVVQINENQGN
SSNNNYSEYRLINCNTSACTQACPKVSFEPIPIHYCAPAGFAILK
CKDEEFNGTGPCKNVSTVQCTHGIKPVVSTQLLLNGSTAEKEIKI
RSENITNNAKIIIVQLVKPVIINCTRPNNNTRRSVHIGPGQAFYA
TGDIIGNIRQAYCTVNRTDWNNTLQQVAKQLGKHFENKTIIFTKS
SGGDLEITTHSFNCGGEFFYCNTSSLFNSTWSHNNSTLLGSNSTE
SNETITLPCRIKQIVNMWQRTGQCMYAPPIKGVIMCVSNITGLIL
TRDGGNDNSTNENETFRPGGGDMRDNWRSELYKYKVVQIEPLGVA
PTRCKRRVVEGGSGGGGSGGGGSGGAVGIGAVFLGFLGAAGSTMG
AASITLTVQARQLLSGIVRQQSNLLRAPEAQQHLLKLTVWGIKQL
QARVLAVERYLRDQQLLGIWGCSGKLICCTSVPWNSSWSNKSLDE
IWENMTWLQWEKEINNYTGLIYSLLEESQNQQEKNEQDLLALDGS
GATNFSLLKQAGDVEENPGPGSGMPMGSLQPLATLYLLGMLVASV
LATDNLWVTVYYGVPVWKDAETTLFCASDAKAYATEKHNVWATHA
CVPTDPNPQEIHLENVTEEFNMWKNNMVEQMHTDIISLWDQSLKP
CVKLTPLCVTLSCSNAKVSYSNATVNNTIQDEIKNCSFNTTTVLR
DKRQKVYSLFYRLDIVQIDNSSSDSSSSEYRLINCNTSACTQACP
KVTFEPIPIHYCAPAGFAILKCKDEEFNGTGPCKNVSTVQCTHGI
KPVVSTQLLLNGSLAKREVKIRSENITNNAKNIIVQFVDPVEINC
TRPNNNTRKSIHIGPGQAFYATGDIIGDIRQAHCNVSRSSWNKTL
QQVAKQLGTYFKNKTIVFNTSSGGDPEITTHSFNCAGEFFYCDTS
GLFNSSWNDTTWKESNSTGSNDTITLLCRIKQIINMWQRTGQCMY
APPIPGLISCKSNITGIILTRDGGNSHRTEETFRPGGGDMRDNWR
SELYRYKVVQIEPLGVAPTRCRRRVVQGGSGGGGSGGGGSGGAVG
IGAVFLGFLGAAGSTMGAASITLTVQARQLLSGIVQQQSNLLRAP
EAQQHLLKLTVWGIKQLQARVLAVERYLRDQQLLGIWGCSGKLIC
CTNVPWNSSWSNKSYNDIWDNMTWLQWDKEIHNYTQLIYNLIEES
QNQQEKNEQDLLALDKWANLWNWFNITNWLWYIKIFIMVVGGLIG
LRIVFAVLSIINRVRQGYSPLSFQTHLPNPRDLDRPERIEEEGGE
QGRDRSIRLVSGFLALAWDDLRSLCLFSYHRLRDFILIAARTVEL
LGQSSLKGLRLGWESLKYLWNLLGYWVRELKISAVNLVDTIAIAV
AGWTDRVIEIGQRIGRAIRHIPRRIRQGLERALL,
SEQ ID NO: 69
Protein sequence, Strain: HT593.1
(DS.SOSIP.664.sc) 2A 5768.04
(DS.SOSIP.664.sc)
MPMGSLQPLATLYLLGMLVASVLATEKLWVTVYYGVPVWKEATTT
LFCASDAKAYETEVHNVWATHACVPTDPNPQEVLLENVTENFNMW
KNNMVEQMQEDIISLWDQSLKPCVKLTPLCVTLECHDVNVNGTAN
NGTTNVTESGVNSSDVTSNNVTNSNWGTMEKGEIKNCSFNITTNI
RDKMQKETAQFYKLDIVPIEDQNKTNNTLYRLINCNTSVCTQACP
KVSFEPIPIHYCTPAGFAILKCNDRNFNGTGPCKNVSTVQCTHGI
KPVVSTQLLLNGSLAEAEVVIRSENFTNNAKTIIIQLNETVEINC
TRPNNNTSKRISIGPGRAFRATKIIGNIRQAHCNISRATWNSTLK
KIVAKLREQFGNKTIVFQPSSGGDPEIVMHSFNCGGEFFYCNTTQ
LFNSTWNSTEESNSTEEGTITLPCRIKQIINMWQEVGKCMYAPPI
EGQIRCSSNITGLLLTRDGGNNNKTNGTEIFRPGGGDMRDNWRSE
LYKYKVVKIEPLGVAPTKCKRRVVQGGSGGGGSGGGGSGGAVGIV
GAMFLGFLGAAGSTMGAASMTLTVQARLLLSGIVQQQNNLLRAPE
AQQHLLQLTVWGIKQLQARVLAVERYLKDQQLLGIWGCSGKLICC
TTVPWNTSWSNKSLSEIWDNMTWMQWEREIDNYTSLIYTLIEESQ
NQQEKNEQELLELDGSGATNFSLLKQAGDVEENPGPGSGMPMGSL
QPLATLYLLGMLVASVLAADKLWVTVYYGVPVWKETTTTLFCASD
ARAYDTEVHNVWATHACVPTDPNPQEVVLGNVTENFNMWKNNMVE
QMHEDIISLWDQSLKPCVRLTPLCVTLNCIDYYGNTTNSNNSSET
MMEKGEIKNCSFNITTRLKDKMQKEYALFYKYDIVPIDNRVGNDT
SNATSYRLTSCNTSVCTQACPKVSFEPIPIHYCAPAGFAILKCND
KKFNGTGPCKNVSTVQCTHGIKPVVSTQLLLNGSLAEEEVMIRSE
NFTDNAKTIIVQLNETVEINCTRPNNNTRKSIHMGPGKVFYTTGE
IIGDIRQAHCNINRAKWNNTLIKIVEKLRVKFNKTISFKQSSGGD
PEIEMHSFNCGGEFFYCNTTQLFNSTWFNNATLNVNSNVTEGSEN
ITLPCRIRQIVNMWQEVGKCMYAPPIQGQIRCSSNITGLLLTRDG
GGSNSSNTSEEVFRPGGGNMRDNWRSELYKYKVVKIEPLGIAPTK
CKRRVVQGGSGGGGSGGGGSGGTVGIGALFLGFLGAAGSTMGAAS
MTLTVQARQLLSGIVQQQNNLLRAPQAQQHLLQLTVWGIKQLQAR
VLAVERYLKDQQLLGIWGCSGKLICCTAVPWNASWSNKSLNEIWD
NMTWMEWEKEIDNYTSLIYTLIEESQNQQEKNEQELLELD,
SEQ ID NO: 70
Protein sequence, Strain: 286.36
(DS.SOSIP.664.sc + Insect Ferritin Heavy
Chain) 2A DU172.17
(DS.SOSIP.sc + Insect Ferritin Light Chain)
MPMGSLQPLATLYLLGMLVASVLAGEDLWVTVYYGVPVWKEANPT
LFCASDAKAYKTEMHNVWATHACVPTDPNPQEMVLENVTEDFNMW
KNGMVEQMHQDIISLWDQSLKPCVKLTPLCVTLNCTEVTRSSNGT
INNNSTEMKNCSFNVTTDLRDKKKKEHALFYRLDIVPLDETNGTS
SEYRLINCNTSTCTQACPKVSFDPIPIHYCAPAGYAILKCKDKKF
NGTGPCKNVSTVQCTHGIKPVVSTQLLLNGSIAEGEIIIRSENLT
NNAKIIIVQLNVTVEINCTRPNNNTRRSIRIGPGQTFYATGEIIG
DIRQAHCNISREKWNRTLQKVEKKLEELFPNKTIHFTSSSGGDLE
ITTHSFNCMGEFFYCNTSALFNNNNDSTNSNITLPCRIRQFINMW
QEVGRCMYAPPIQGVITCKSNVTGLLLTRDGGIINDTEIFRPGGG
DMRDNWRSELYKYKVVEIKPLGIAPTTCKRRVVEGGSGGGGSGGG
GSGGAVGIGAVFLGFLGAAGSTMGAASITLTAQARQLLSGIVQQQ
SNLLRAPEAQQHMLQLTVWGIKQLQTRVLAIERYLKDQQLLGIWG
CSGKLICCTAVPWNGSWSNKSQDEIWHNMTWMQWDKEINNYTNII
YGLLEVSQNQQEKNEQDLLALDGGSGGRSCRNSMRQQIQMEVGAS
LQYLAMGAHFSKDVVNRPGFAQLFFDAASEEREHAMKLIEYLLMR
GELTNDVSSLLQVRPPTRSSWKGGVEALEHALSMESDVTKSIRNV
IKACEDDSEFNDYHLVDYLTGDFLEEQYKGQRDLAGKASTLKKLM
DRHEALGEFIFDKKLLGIDVGSGATNFSLLKQAGDVEENPGPGSG
MKAKLLVLLCTFTATYAGNLWVTVYYGVPVWKEAKTTLFCASDAK
AHKEEVHNIWATHACVPTDPNPQEIVLKNVTENFNMWKNDMVDQM
HEDIISLWDQSLKPCVKLTPLCVTLNCSDVKIKGTNATYNNATYN
NNNTISDMKNCSFNTTTEITDKKKKEYALFYKLDVVALDGKETNS
TNSSEYRLINCNTSACTQACPKVSFDPIPIHYCAPAGYAILKCNN
KTFNGTGPCNNVSTVQCTHGIKPVVSTQLLLNGSLAEEEVVIRFE
NLTNNAKIIIVHLNESVEINCTRPSNNTRKSVRIGPGQTFFATGD
IIGDIRQAHCNISRKKWNTTLQRVKEKLKEKFPNKTIQFAPSSGG
DLEITTHSFNCRGEFFYCYTSDLFNSTYMSNNTGGANITLQCRIK
QIIRMWQGVGQCMYAPPIAGNITCKSNITGLLLTRDGGKEKNDTE
TFRPGGGDMRDNWRSELYKYKVVEIKPLGIAPDKCKRRVVEGGSG
GGGSGGGGSGGAVGIGAVFLGFLGAAGSTMGAASMTLTVQARQLL
SGIVQQQSNLLRAPEAQQHMLQLTVWGIKQLQTRVLAIERYLKDQ
QLLGIWGCSGKLICCTAVPWNASWSNKSYEEIWGNMTWMQWDREI
NNYTNTIYSLLEESQNQQEKNEKDLLALDGGSGGEYGSHGNVATE
LQAYAKLHLERSYDYLLSAAYFNNYQTNRAGFSKLFKKLSDEAWS
KTIDIIKHVTKRGDKMNFDQHSTMKTERKNYTAENHELEALAKAL
DTQKELAERAFYIHREATRNSQHLHDPEIAQYLEEEFIEDHAEKI
RTLAGHTSDLKKFITANNGHDLSLALYVFDEYLQKTV,
SEQ ID NO: 71
Protein sequence, Strain: MB539.2B7
DS.SOSIP.664.sc + Insect Ferritin
Heavy Chain) 2A KNH1209.18
(DS.SOSIP.664.sc + Insect Ferritin Light Chain)
MPMGSLQPLATLYLLGMLVASVLAAENLWVTVYYGVPVWRDADTT
LFCASDAKAYETEKHNVWATHACVPTDPNPQEIDLKNVTEEFNMW
KNNMVEQMHTDIISLWDQSLKPCVKLTPLCVTLNCSNANVTSENS
TIMGDREEIKNCSFNMTTELRDKRQKVYSLFYRLDVVQINENQGN
SSNNNYSEYRLINCNTSACTQACPKVSFEPIPIHYCAPAGFAILK
CKDEEFNGTGPCKNVSTVQCTHGIKPVVSTQLLLNGSTAEKEIKI
RSENITNNAKIIIVQLVKPVIINCTRPNNNTRRSVHIGPGQAFYA
TGDIIGNIRQAYCTVNRTDWNNTLQQVAKQLGKHFENKTIIFTKS
SGGDLEITTHSFNCGGEFFYCNTSSLFNSTWSHNNSTLLGSNSTE
SNETITLPCRIKQIVNMWQRTGQCMYAPPIKGVIMCVSNITGLIL
TRDGGNDNSTNENETFRPGGGDMRDNWRSELYKYKVVQIEPLGVA
PTRCKRRVVEGGSGGGGSGGGGSGGAVGIGAVFLGFLGAAGSTMG
AASITLTVQARQLLSGIVRQQSNLLRAPEAQQHLLKLTVWGIKQL
QARVLAVERYLRDQQLLGIWGCSGKLICCTSVPWNSSWSNKSLDE
IWENMTWLQWEKEINNYTGLIYSLLEESQNQQEKNEQDLLALDGG
SGGRSCRNSMRQQIQMEVGASLQYLAMGAHFSKDVVNRPGFAQLF
FDAASEEREHAMKLIEYLLMRGELTNDVSSLLQVRPPTRSSWKGG
VEALEHALSMESDVTKSIRNVIKACEDDSEFNDYHLVDYLTGDFL
EEQYKGQRDLAGKASTLKKLMDRHEALGEFIFDKKLLGIDVGSGA
TNFSLLKQAGDVEENPGPGSGMPMGSLQPLATLYLLGMLVASVLA
TDNLWVTVYYGVPVWKDAETTLFCASDAKAYATEKHNVWATHACV
PTDPNPQEIHLENVTEEFNMWKNNMVEQMHTDIISLWDQSLKPCV
KLTPLCVTLSCSNAKVSYSNATVNNTIQDEIKNCSFNTTTVLRDK
RQKVYSLFYRLDIVQIDNSSSDSSSSEYRLINCNTSACTQACPKV
TFEPIPIHYCAPAGFAILKCKDEEFNGTGPCKNVSTVQCTHGIKP
VVSTQLLLNGSLAKREVKIRSENITNNAKNIIVQFVDPVEINCTR
PNNNTRKSIHIGPGQAFYATGDIIGDIRQAHCNVSRSSWNKTLQQ
VAKQLGTYFKNKTIVFNTSSGGDPEITTHSFNCAGEFFYCDTSGL
FNSSWNDTTWKESNSTGSNDTITLLCRIKQIINMWQRTGQCMYAP
PIPGLISCKSNITGIILTRDGGNSHRTEETFRPGGGDMRDNWRSE
LYRYKVVQIEPLGVAPTRCRRRVVQGGSGGGGSGGGGSGGAVGIG
AVFLGFLGAAGSTMGAASITLTVQARQLLSGIVQQQSNLLRAPEA
QQHLLKLTVWGIKQLQARVLAVERYLRDQQLLGIWGCSGKLICCT
NVPWNSSWSNKSYNDIWDNMTWLQWDKEIHNYTQLIYNLIEESQN
QQEKNEQDLLALDGGSGGEYGSHGNVATELQAYAKLHLERSYDYL
LSAAYFNNYQTNRAGFSKLFKKLSDEAWSKTIDIIKHVTKRGDKM
NFDQHSTMKTERKNYTAENHELEALAKALDTQKELAERAFYIHRE
ATRNSQHLHDPEIAQYLEEEFIEDHAEKIRTLAGHTSDLKKFITA
NNGHDLSLALYVFDEYLQKTV,

1. A recombinant nucleic acid comprising two or more polynucleotide sequences encoding two or more antigens, wherein the 3′ end of each of the two or more polynucleotide sequences encoding the two or more antigens is operably linked to a 2A polynucleotide sequence.

2. The recombinant nucleic acid of claim 1, wherein the 2A polynucleotide sequence encodes a 2A polypeptide that is self-cleavage.

3. The recombinant nucleic acid of claim 1, wherein the 5′ end of each of the two or more polynucleotides encoding the two or more antigens is operably linked to a polynucleotide sequence encoding a signal peptide.

4. The recombinant nucleic acid of claim 1, wherein the two or more antigens are antigens of pathogens.

5. The recombinant nucleic acid of claim 4, wherein the antigens are viral antigens.

6. The recombinant nucleic acid of claim 5, wherein the viral antigens are HIV antigens, influenza antigens, or SARS-CoV-2 antigens.

7. The recombinant nucleic acid of claim 6, wherein the HIV antigens are HIV Env proteins or HIV fusion peptides.

8. The recombinant nucleic acid of claim 6 or 7, wherein the polynucleotide sequence encoding the HIV antigen comprises a sequence at least about 90% identical to SEQ ID NO: 5 or 7.

9. The recombinant nucleic acid of claim 1, wherein the polynucleotide sequence encoding the signal peptide comprises a sequence at least about 90% identical to SEQ ID NO: 15.

10. The recombinant nucleic acid of claim 1, wherein the 2A polynucleotide sequence comprises a sequence at least about 90% identical to SEQ ID NO: 11 or 12.

11. The recombinant nucleic acid of claim 1, wherein the polynucleotide sequence encoding the signal peptide, the polynucleotide sequence encoding the antigen, and the 2A polynucleotide sequence are operably linked.

12. The recombinant nucleic acid of claim 1, further comprising a polynucleotide sequence encoding a ferritin protein.

13. The recombinant nucleotide of claim 12, wherein the polynucleotide sequence encoding the ferritin protein is operably linked to the 3′ end of each of the two or more of the polynucleotide sequences encoding the two or more antigens and to the 5′ end of the 2A polynucleotide sequence.

14. The recombinant nucleic acid of claim 1, wherein the recombinant nucleic acid comprises a sequence at least about 90% identical to SEQ ID NO: 1 or 3.

15. A DNA vaccine comprising the recombinant nucleic acid of claim 1.

16. An RNA vaccine comprising a sequence that is transcribed from the recombinant nucleic acid of claim 1.

17. A recombinant nucleic acid comprising two or more polynucleotide sequences encoding two or more antigens, wherein the 3′ end of each of the two or more polynucleotide sequences encoding the two or more antigens is operably linked to a polynucleotide sequence encoding a ferritin protein and a 2A polynucleotide sequence.

18.-26. (canceled)

27. The recombinant nucleic acid of claim 17, wherein the polynucleotide sequence encoding the ferritin protein comprises a sequence at least about 90% identical to SEQ ID NO: 9.

28. (canceled)

29. (canceled)

30. A nanoparticle vaccine encoded by the recombinant nucleic acid of claim 17.

31. A method of preventing and/or treating HIV infection in a subject, comprising administering to the subject an effective amount of the nanoparticle vaccine of claim 30.
